(19) United States
(12) Patent Application Publication
(10) Pub. No.: US 2017/0273985 A1
(43) Pub. Date: Sep. 28, 2017

(54) TRICYCLIC DERIVATIVE

(71) Applicants: Sunovion Pharmaceuticals Inc., Marlborough, MA (US); Sumitomo Dainippon Pharma Co., Ltd., Osaka-Shi, Osaka (JP)

(72) Inventors: Douglas F. Burdi, Arlington, MA (US); Daisuke Tanaka, Ibaraki-shi, Osaka (JP); Yuki Fujii, Osaka-Shi, Osaka (JP); Muneo Kawasumi, Kanazawa-Shi, Ishikawa (JP)

(21) Appl. No.: 15/511,954

(22) PCT Filed: Sep. 17, 2015
(86) PCT No.: PCT/JP2015/004781

§ 371 Date: Mar. 16, 2017

Related U.S. Application Data
(60) Provisional application No. 62/052,362, filed on Sep. 18, 2014.

(51) Int. Cl. A61K 31/519; A61K 31/53; C07D 498/14; C07D 487/14

(52) U.S. Cl. CPC A61K 31/519; C07D 487/14; A61K 31/53; C07D 498/14

(57) ABSTRACT

Disclosed are compounds useful as inhibitors of phosphodiesterase 1 (PDE1), compositions thereof, and methods of using the same.

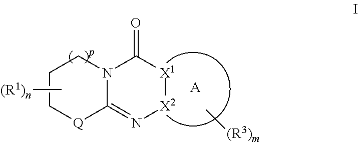

### TECHNICAL FIELD

The present invention mainly relates to compounds useful as inhibitors of phosphodiesterase 1 (PDE1).

### BACKGROUND ART

The prevalence of neurological and psychiatric disorders is increasing worldwide. Up to one billion people suffer from debilitating neurological conditions such as Alzheimer's disease and Parkinson's disease, with almost seven million people dying every year. “Neurological disorders: public health challenges” World Health Organization, 2006. Neurological and psychiatric disorders are prevalent in all countries, often without regard to age, sex, education or income. However, as many neurological disorders are correlated with increased age, as the global population ages, the impact of these disorders becomes more evident.

Despite the availability of treatments for some of these diseases, first line therapies (such as L-DOPA for Parkinson's) are often burdened by unfavorable side effects, or may lack efficacy. For instance, there is currently no approved treatment for the cognitive deficits in schizophrenia despite the high unmet medical need.

The continuing and increasing problem of neurological and psychiatric disorders, and the current lack of safe and effective drugs for treating them, highlight the overwhelming need for new drugs to treat these conditions and their underlying causes.

### SUMMARY OF INVENTION

It has now been found that compounds of this invention, and pharmaceutically acceptable compositions thereof, are effective as inhibitors of Phosphodiesterase 1 (PDE1) enzymes. Such compounds have the general formula I:

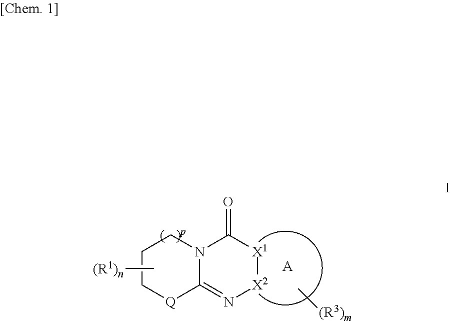

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of PDE1 enzymes. Such diseases, disorders, or conditions include those described herein.

Compounds provided by this invention are also useful for the study of PDE1 enzymes in biological and pathological phenomena; the study of intracellular signal transduction pathways occurring in PDE1-expressing tissues; and the comparative evaluation of new PDE1 inhibitors or other regulators neuronal activity in vitro or in vivo.

### DESCRIPTION OF EMBODIMENTS

### 1. General Description of Compounds of the Invention

In certain embodiments, the present invention provides inhibitors of PDE1. In some embodiments, such compounds include those of formula I:

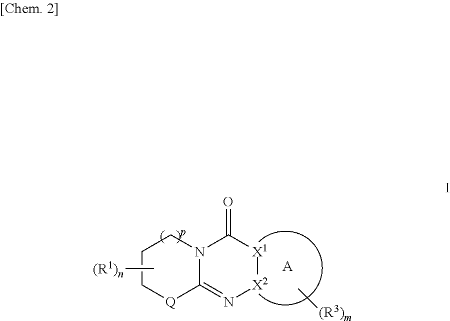

or a pharmaceutically acceptable salt thereof, wherein:

Q is —N(L¹-R²)—, —C(R⁴)₂—, —O—, or —S—;

X¹ and X² are each independently C or N;

Ring A is a 5-6 membered heteroaryl ring;

L¹ is a covalent bond, or a C₁₋₆ bivalent straight or branched hydrocarbon chain, wherein one or more hydrogen atoms of the chain are optionally substituted with the same or different 1 to 4 group(s) selected from

(a) a halogen,

(b) a hydroxy,

(c) a C₁₋₆ alkoxy (said group being optionally substituted with the same or different 1 to 3 halogen), and

(d) an oxo,

each R¹ and R³ are independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, C(O)R, —C(O)OR, —OC(O)R, —S(O)R, or —S(O)₂R;

each R is independently
(i) a hydrogen,
(ii) a C₁₋₆ aliphatic (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo), or
(iii) a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said group is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano;
R² is selected from
(i) a hydrogen,
(ii) a halogen,
(iii) a hydroxy,
(iv) a cyano,
(v) a C₁₋₆ alkoxy (said group being optionally substituted with the same or different 1 to 3 halogen or hydroxy), or
(vi) a 3-8 membered saturated or partially unsaturated monocyclic carbocyclic ring; a phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with one or more R⁵;
provided that when L¹ is a covalent bond, R² is not hydrogen;
each R⁴ is independently —R;
each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, or —S(O)R; wherein one or more of {an R¹ and an R²}, {R¹ and an R⁴}, {two instances of R¹} and {two instances of R³} may be taken together with their intervening atoms to form a ring, substituted with q instances of R⁵; wherein said ring is a 3-8 membered saturated or partially unsaturated monocyclic carbocyclic ring; or a 4-8 membered saturated or partially unsaturated monocyclic heterocyclic ring;
m is 0-4;
n is 0-4;
p is 0-2; and
q is 0-5.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, boron, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆ or C₁₋₄) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₜ—, wherein t is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic or bicyclic ring systems having a total of five to fourteen ring members, wherein at least one ring in the system is a carbocyclic aromatic ring and wherein each ring in the system contains 3 to 7 ring members. The term “aryl” may be used interchangeably with the term “aryl ring”. In certain embodiments of the present invention, “aryl” refers to a carbocyclic aromatic ring system which includes, but not limited to, phenyl, naphthyl, anthryl and the like, which may be optionally substituted. Also included within the scope of the term “aryl”, as it is used herein, is a group in which a carbocyclic aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, 9 or 10 ring atoms; having 6, 10, or 14 ir electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Description of Exemplary Embodiments

In certain embodiments, the present invention provides inhibitors of PDE1. In some embodiments, such compounds include those of formula I:

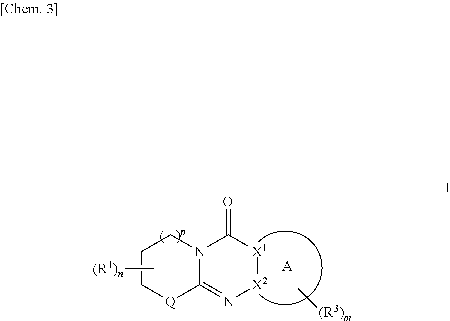

or a pharmaceutically acceptable salt thereof, wherein:

Q is —N(L¹-R²)—, —C(R⁴)₂—, —O—, or —S—;

X¹ and X² are each independently C or N;

Ring A is a 5-6 membered heteroaryl ring;

L¹ is a covalent bond, or a C₁₋₆ bivalent straight or branched hydrocarbon chain, wherein one or more hydrogen atoms of the chain are optionally substituted with the same or different 1 to 4 group(s) selected from

(a) a halogen,

(b) a hydroxy,

(c) a C₁₋₆ alkoxy (said group being optionally substituted with the same or different 1 to 3 halogen), and

(d) an oxo;

each R¹ and R³ are independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, C(O)R, —C(O)OR, —OC(O)R, —S(O)R, or —S(O)₂R;

each R is independently

(i) a hydrogen,

(ii) a C₁₋₆ aliphatic (said group being optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) an oxo), or

(iii) a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, each of said group is optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) a cyano;

R² is

(i) a hydrogen,

(ii) a halogen,

(iii) a hydroxy,

(iv) a cyano,

(v) a C₁₋₆ alkoxy (said group being optionally substituted with the same or different 1 to 3 halogen or hydroxy), or

(vi) a 3-8 membered saturated or partially unsaturated monocyclic carbocyclic ring; a phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with one or more R⁵;

provided that when L¹ is a covalent bond, R² is not hydrogen;

each R⁴ is independently —R;

each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, or —S(O)R; wherein one or more of {an R¹ and an R²}, {R¹ and an R⁴}, {two instances of R¹} and {two instances of R³} may be taken together with their intervening atoms to form a ring, substituted with q instances of R⁵; wherein said ring is a 3-8 membered saturated or partially unsaturated monocyclic carbocyclic ring; or a 4-8 membered saturated or partially unsaturated monocyclic heterocyclic ring;

m is 0-4;

n is 0-4;

p is 0-2; and

q is 0-5.

In another embodiment, the present invention can provide the above-defined compounds of formula I wherein L¹ is a covalent bond and R² is hydrogen, i.e., the proviso of “when L¹ is a covalent bond, R² is not hydrogen” in the above definition of the compounds of formula I may be deleted.

As defined generally above, Q is —N(L¹-R²)—, —C(R⁴)₂—, —O—, or —S—. In certain embodiments, Q is —N(L¹-R²)—. In certain embodiments, Q is —O—. In certain embodiments, Q is —C(R⁴)₂—. In certain embodiments, Q is —CH(R⁴)—. In certain embodiments, Q is —CH₂—. In certain embodiments, Q is —S—. In certain embodiments, Q is —NH—. In certain embodiments, Q is —N(L¹-R²)—.

As defined generally above, X¹ and X² are each independently C or N. In some embodiments, X¹ is C, and X² is N. In some embodiments, X¹ is N, and X² is C. In some embodiments both of X¹ and X² are C.

As defined generally above, Ring A is a 5-6 membered heteroaryl ring. In some embodiments, Ring A is a 5-6 membered heteroaryl ring having 1-4 heteroatoms independently selected from nitrogen, oxygen and sulfur. In some embodiments, Ring A is pyrrolo. In some embodiments, Ring A is furano. In some embodiments, Ring A is thieno. In some embodiments, Ring A is pyrazolo. In some embodiments, Ring A is imidazolo. In some embodiments, Ring A is oxazolo. In some embodiments, Ring A is isoxazolo. In some embodiments, Ring A is thiazolo. In some embodiments, Ring A is isothiazolo. In some embodiments, Ring A is triazolo. In some embodiments, Ring A is tetrazolo. In some embodiments, Ring A is pyridino. In some embodiments, Ring A is pyrimidino. In some embodiments, Ring A is pyridizino. In some embodiments, Ring A is selected from pyrazolo and imidazolo. In some embodiments, Ring A is not pyrrolo, thieno, or furano.

As defined generally above, each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, C(O)R, —C(O)OR, —OC(O)R, —S(O)R, or —S(O)₂R. In some embodiments, R¹ is —R. In some embodiments, R¹ is selected from

(i) a hydrogen,

(ii) a halogen,

(iii) a C₃₋₇ cycloaliphatic; phenyl; a 5 or 6-membered monocyclic heteroaryl, a C₁ 4 alkyl-phenyl, or a C₁₋₄ alkyl-5 or 6-membered monocyclic heteroaryl, wherein each of said groups is optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) a cyano, or

(iv) a C₁₋₆ alkyl (said group being optionally substituted with the same or different 1 to 3 halogen).

In some embodiments, R¹ is a phenyl (said group being optionally substituted with the same or different 1 to 4 group(s) selected from the group consisting of

(a) a halogen,

(b) a C₁₋₆ alkyl (said group being optionally substituted with the same or different 1 to 3 halogen), and

(c) a C₁₋₆ alkoxy (said group being optionally substituted with the same or different 1 to 3 halogen).

In some embodiments, R¹ is a halogen. In some embodiments, R¹ is a C₁₋₆ alkyl (said group being optionally substituted with the same or different 1 to 3 halogen). In some embodiments, two instances of R¹ may be taken together with their intervening atoms to form a 3-6 membered saturated monocyclic carbocyclic ring.

As defined generally above, each R³ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, C(O)R, —C(O)OR, —OC(O)R, —S(O)R, or —S(O)₂R. In some embodiments, R³ is —R. In some embodiments, R³ is selected from

(i) a hydrogen,

(ii) a halogen,

(iii) a C₁₋₆ aliphatic (said group being optionally substituted with the same or different 1 to 4 group(s) selected from the group consisting of

(a) a halogen,

(b) a C₁₋₆ alkoxy (said group being optionally substituted with the same or different 1 to 3 halogen),

(c) a hydroxy, and

(d) an oxo),

(iv) a 4-8 membered saturated or partially unsaturated monocyclic heterocyclyl (said group being optionally substituted with the same or different 1 to 4 group(s) selected from the group consisting of

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) a cyano), or

(v) a cyano.

In some embodiments, R³ is a C₁₋₆ alkyl. In some embodiments, R³ is a 3-8 membered saturated or partially unsaturated monocyclic carbocyclic ring. In some embodiments, R³ is a 4-8 membered saturated or partially unsaturated monocyclic heterocyclyl. In some embodiments, R³ is tetrahydropyranyl or tetrahydrofuranyl. In some embodiments, R³ is a halogen. In some embodiments, R³ is a cyano.

As defined generally above, L¹ is a covalent bond, or a C₁₋₆ bivalent straight or branched hydrocarbon chain, wherein one or more hydrogen atoms of the chain are optionally substituted with the same or different 1 to 4 group(s) selected from the group consisting of

(a) a halogen,

(b) a hydroxy,

(c) a C₁₋₆ alkoxy (said group being optionally substituted with the same or different 1 to 3 halogen), and

(d) an oxo.

In some embodiments, L¹ is a covalent bond. In some embodiments, L¹ is a C₁₋₄ bivalent straight or branched hydrocarbon chain, wherein one or more hydrogen atoms of the chain are optionally and independently replaced by halogen. In some embodiments, L¹ is a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —C(O)O—, —OC(O)—, —C(O)—, —O—, —S—, —S(O)— or —S(O)₂—. In some embodiments, L¹ is a C₁₋₆ bivalent straight or branched hydrocarbon chain (said group being optionally substituted with an oxo). In some embodiments, L¹ is a C₁₋₄ bivalent straight hydrocarbon chain, wherein one methylene unit of the chain is replaced by —O—. In some embodiments, L¹ is a C₁₋₆ bivalent straight or branched hydrocarbon chain. In some embodiments, L¹ is selected from methylene, ethylene, propylene and butylene. In some embodiments, L¹ is methylene. In some embodiments, L¹ is —O—. In some embodiments, L¹ is —S—, —S(O)—, or —S(O)₂—.

As defined generally above, R² is

(i) a hydrogen,

(ii) a halogen,

(iii) a hydroxy,

(iv) a cyano,

(v) a C₁₋₆ alkoxy (said group being optionally substituted with the same or different 1 to 3 halogen or hydroxy), or

(vi) a 3-8 membered saturated or partially unsaturated monocyclic carbocyclic ring; a phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with one or more R⁵; In some embodiments, R² is a C₃₋₇ cycloaliphatic; a phenyl; a 5-6 membered monocyclic heteroaryl, or a 4-8 membered saturated or partially unsaturated monocyclic heterocyclyl, each of said group is optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy,

(e) a cyano, and

(f) a 5-6 membered monocyclic heteroaryl (said group being optionally substituted with the same or different 1 to 3 halogen).

In some embodiments, R² is a hydrogen or a C₃₋₇ cycloalkyl (said group being optionally substituted with the same or different 1 to 4 halogen, C₁₋₆ alkyl (said group being optionally substituted with the same or different 1 to 3 halogen), or C₁₋₆ alkoxy (said group being optionally substituted with the same or different 1 to 3 halogen)).

As defined generally above, each R⁴ is independently —R. In some embodiments, each R⁴ is hydrogen. In some embodiments, at least one R⁴ is not hydrogen. In some embodiments, one R⁴ is C₁₋₆ aliphatic and one R⁴ is hydrogen. In some embodiments, one R⁴ is C₁₋₆ alkyl and one R⁴ is hydrogen. In some embodiments, each R⁴ is C₁₋₆ aliphatic. In some embodiments, each R⁴ is C₁₋₆ alkyl.

As defined generally above, each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, or —S(O)R. In some embodiments, each R⁵ is independently halogen, —R, —CN, or —OR. In some embodiments, each R⁵ is independently halogen, phenyl, methyl, ethyl, trifluoromethyl, —CN, methoxy, ethoxy, propoxy, or isopropoxy.

As defined generally above one or more of {an R¹ and an R²}, {R¹ and an R⁴}, {two instances of R¹} and {two instances of R³} may be taken together with their intervening atoms to form a ring, substituted with q instances of R⁵; wherein said ring is a 3-8 membered saturated or partially unsaturated monocyclic carbocyclic ring; or a 4-8 membered saturated or partially unsaturated monocyclic heterocyclic ring. In some embodiments, {an R¹ and an R²}, {R¹ and an R⁴}, {two instances of R¹} and {two instances of R³} may be taken together with their intervening atoms to form a ring, wherein said ring is a 3-8 membered saturated or partially unsaturated monocyclic carbocyclic ring; or a 4-8 membered saturated or partially unsaturated monocyclic heterocyclic ring having 1-2 heteroatoms independently selected from nitrogen, oxygen, and sulfur, and wherein said ring is substituted with q instances of R⁵. In some embodiments, none of {an R¹ and an R²}, {R¹ and an R⁴}, {two instances of R¹} and {two instances of R³} are taken together with their intervening atoms to form a ring.

As defined generally above, m is 0-4. In some embodiments, m is 0-1. In some embodiments, m is 1.

As defined generally above, n is 0-4. In some embodiments, n is 0-1. In some embodiments, n is 0. In some embodiments, n is 1.

As defined generally above, p is 0-2. In some embodiments, p is 0-1. In some embodiments, p is 0. In some embodiments, p is 1.

As defined generally above, q is 0-5. In some embodiments, q is 0. In some embodiments, q is 1-5. In some embodiments, q is 1-2. In some embodiments, q is 1. In some embodiments, q is 2. In some embodiments, q is 3.

In some embodiments, the present invention provides a compound of formula I selected from formulas I-a, I-b, and I-c:

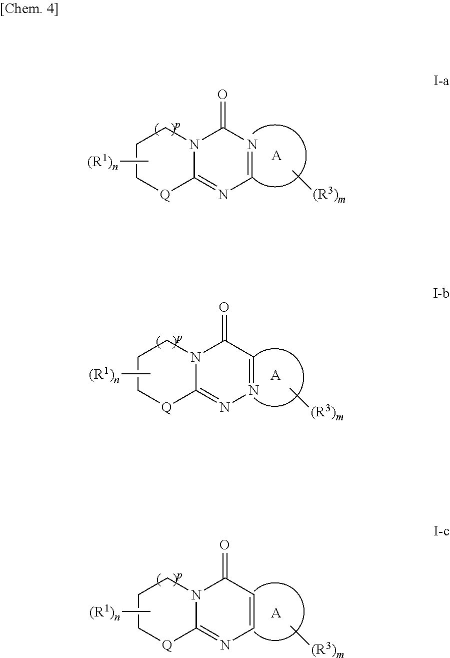

or a pharmaceutically acceptable salt thereof; wherein each of Q, Ring A, R¹, R³, p, n, and m is as described in embodiments for formula I, supra, or described in embodiments herein, both singly and in combination.

In certain embodiments, the present invention provides a compound of formula I selected from formulas II-a, II-b, II-c, II-d, II-e, II-f, II-g, II-h, II-i, II-j, II-k, II-1, II-m and II-n:

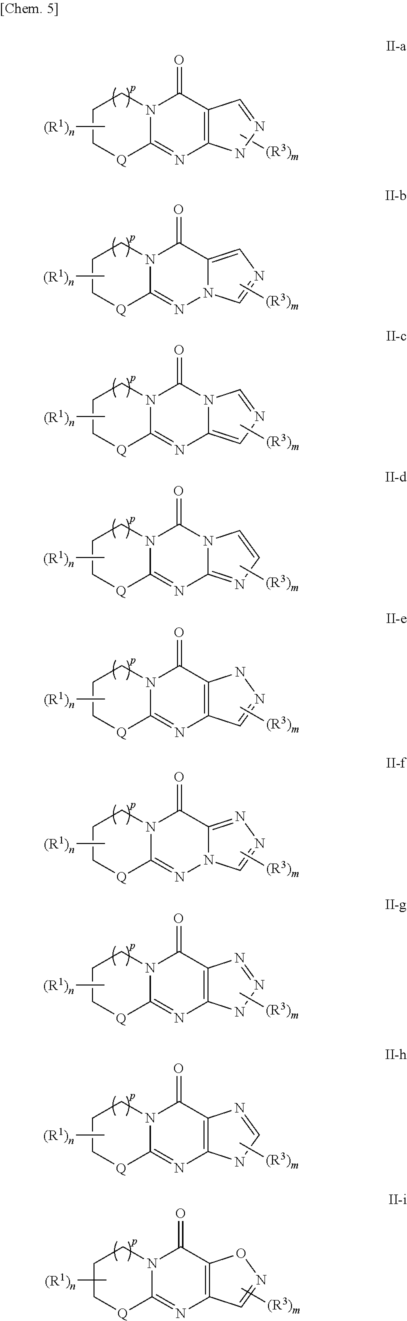

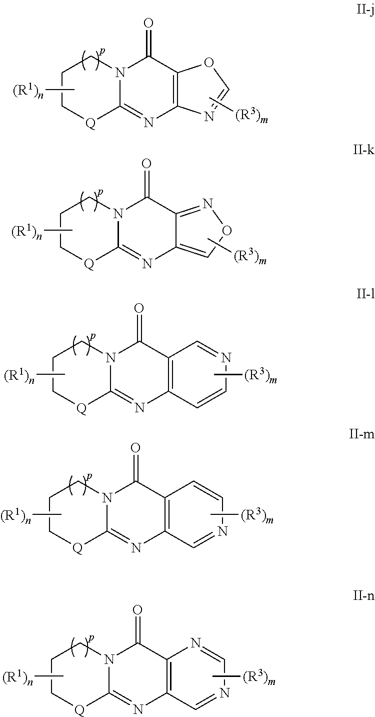

or a pharmaceutically acceptable salt thereof, wherein:
each of Q, R¹, R³, p, n, and m is as described in embodiments for formula I, supra, or described in embodiments herein, both singly and in combination.

In some embodiments, the present invention provides a compound of formula I selected from formulas III-a, III-b and III-n:

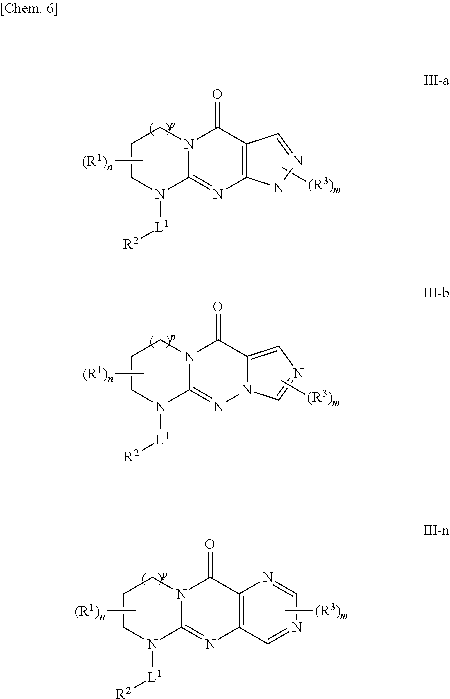

or a pharmaceutically acceptable salt thereof; wherein each of L¹, R¹, R², R³, p, n, and m is as described in embodiments for formula I, supra, or described in embodiments herein, both singly and in combination.

In certain embodiments, the present invention provides any compound selected from those depicted in the Examples disclosed herein, or a pharmaceutically acceptable salt thereof.

### 4. Uses. Formulation and Administration and Pharmaceutically Acceptable Compositions

According to another embodiment, the invention provides a composition comprising a compound of this invention or a pharmaceutically acceptable salt, ester, or salt of ester thereof and a pharmaceutically acceptable carrier, adjuvant, or vehicle. The amount of compound in compositions of this invention is such that is effective to measurably inhibit PDE1, in a biological sample or in a patient. In certain embodiments, the amount of compound in compositions of this invention is such that is effective to measurably inhibit PDE1, in a biological sample or in a patient. In certain embodiments, a composition of this invention is formulated for administration to a patient in need of such composition. In some embodiments, a composition of this invention is formulated for oral administration to a patient.

The term “patient”, as used herein, means an animal, preferably a mammal, and most preferably a human.

The term “pharmaceutically acceptable carrier, adjuvant, or vehicle” refers to a nontoxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of PDE1.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceuticallptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon a variety of factors, including the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Phosphodiesterases (PDE's) are enzymes that catalyze the hydrolysis of the cyclic phosphate bonds of cyclic guanosine monophosphate (cGMP) and/or cyclic adenosine monophosphate (cAMP). Lugnier, C., Pharmacology & Therapeutics (2006), 109, 366. The PDE superfamily can be grouped into 11 families (PDE1-11) based on their sequence, regulation and substrate specificity. Each family can contain multiple subtypes, each the product of individual genes. In particular, the PDE1 family, consisting of PDE1A, PDE1B and PDE1C, are so-called dual substrate enzymes that hydrolyze both cGMP and cAMP, and are regulated by Ca²⁺ and calmodulin. PDE1A is expressed throughout the brain, especially in the hippocampus and cerebellum, and at lower levels in the striatum, as well as in the peripheral vasculature. PDE1B, by contrast, is expressed primarily in the striatum and cerebellum, and is often found in regions of high dopaminergic tone and dopamine D1 receptor expression. PDE1C is primarily expressed in the heart, olfactory epithelium and striatum. Considering these expression patterns, a compound that is selective for PDE1B over PDE1A and/or PDE1C may have fewer effects on the cardiovascular system.

Due to the expression pattern of the PDE1 family, inhibition of PDE1 may be useful in the treatment of disorders involving learning and memory by enhancing neuronal plasticity. The increased levels of intracellular cAMP and cGMP caused by PDE1 inhibition trigger cascades that ultimately lead to the phosphorylation and activation of the transcription factors cAMP Responsive Element Binding Protein (CREB) and Serum Response Factor (SRF). Josselyn, S. A., Nguyen, P. V., Current Drug Targets—CNS & Neurological Disorders (2005) 4, 481. Activation of CREB and SRF can lead to the expression of plasticity-related genes which mediate the processes that are critical for neuronal plasticity such as the remodeling of dendritic spines. PDE1 inhibitors may therefore be useful in the treatment of cognitive symptoms of disorders such as Alzheimer's Disease, Parkinson's Disease, Stroke, Schizophrenia, Down Syndrome, Fetal Alcohol Syndrome and others.

Due to its location in the striatum and its role in modulating levels of secondary messengers such as cyclic nucleotides, PDE1 is also a regulator of locomotor activity. Reed, T. M. J., et al., Journal of Neuroscience (2002) 22, 5189). Due to their ability to increase levels of cyclic nucleotides in the striatum, PDE1 inhibitors are expected to potentiate the effects of D1 agonists by inhibiting the degradation of cAMP and cGMP. This potentiation of dopamine signaling may be useful in the treatment of diseases including, but not limited to Parkinson's Disease, depression and cognitive disorders including Cognitive Impairment Associated with Schizophrenia.

The activity of a compound utilized in this invention as an inhibitor of PDE1 or a treatment for a neurological or psychiatric disorder, may be assayed in vitro or in vivo. An in vivo assessment of the efficacy of the compounds of the invention may be made using an animal model of a neurological or psychiatric disorder, e.g., a rodent or primate model. Cell-based assays may be performed using, e.g., a cell line isolated from a tissue that expresses PDE1, or a cell line that recombinantly expresses PDE1. Additionally, biochemical or mechanism-based assays, e.g., measuring cAMP or cGMP levels, Northern blot, RT-PCR, etc., may be performed. In vitro assays include assays that determine cell morphology, protein expression, and/or the cytotoxicity, enzyme inhibitory activity, and/or the subsequent functional consequences of treatment of cells with compounds of the invention. Alternate in vitro assays quantify the ability of the inhibitor to bind to protein or nucleic acid molecules within the cell. Inhibitor binding may be measured by radiolabelling the inhibitor prior to binding, isolating the inhibitor/target molecule complex and determining the amount of radiolabel bound. Alternatively, inhibitor binding may be determined by running a competition experiment where new inhibitors are incubated with purified proteins or nucleic acids bound to known radioligands. Detailed conditions for assaying a compound utilized in this invention as an inhibitor of PDE1 are set forth in the Examples below. The aforementioned assays are exemplary and not intended to limit the scope of the invention. The skilled practitioner can appreciate that modifications can be made to conventional assays to develop equivalent assays that obtain the same result.

As used herein, the terms “treatment”, “treat”, and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds and compositions, according to the method of the present invention, may be administered using any amount and any route of administration effective for treating or lessening the severity of a neurological or psychiatric disorder.

In some embodiments, the compounds and compositions, according to the method of the present invention, may be administered using any amount and any route of administration effective for treating or lessening the severity of a disease associated with PDE1.

In some embodiments, the compounds and compositions, according to the method of the present invention, may be administered using any amount and any route of administration effective for treating or lessening the severity of a neurological or psychiatric disorder.

In some embodiments, the neurological or psychiatric disorder is selected from schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosispsychotic disorder, psychosis associated with affective disorders, brief reactive psychosis, schizoaffective psychosis, “schizophreniaspectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Down syndrome, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, Parkinsonism-ALS dementia complex and basal ganglia calcification), medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias {including drug e.g. L-DOPA induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)}; urinary incontinence; neuronal damage including ocular damage, retinopathy or macular degeneration of the eye, tinnitus, hearing impairment and loss, and brain edema; emesis; and sleep disorders including insomnia and narcolepsy

In some embodiments, the neurological or psychiatric disorder is selected from the group consisting of Alzheimer's Disease, Parkinson's Disease, depression, cognitive impairment, stroke, schizophrenia, Down Syndrome, and Fetal Alcohol Syndrome. In some embodiments, the neurological or psychiatric disorder is Alzheimer's Disease. In some embodiments, the neurological or psychiatric disorder is Parkinson's Disease. In some embodiments, the neurological or psychiatric disorder is depression. In some embodiments, the neurological or psychiatric disorder is cognitive impairment. In some embodiments, the neurological or psychiatric disorder is stroke. In some embodiments, the neurological or psychiatric disorder is schizophrenia. In some embodiments, the neurological or psychiatric disorder is Down Syndrome. In some embodiments, the neurological or psychiatric disorder is Fetal Alcohol Syndrome.

In some embodiments, the neurological or psychiatric disorder involves a deficit in cognition (cognitive domains as defined by the Diagnostic and Statistical Manual of Mental Disorders, 5th Ed., American Psychiatric Publishing (2013) (“DSM-5”) are: complex attention, executive function, learning and memory, language, perceptualmotor, social cognition). In some embodiments, the neurological or psychiatric disorder is associated with a deficit in dopamine signaling. In some embodiments, the neurological or psychiatric disorder is associated with basal ganglia dysfunction. In some embodiments, the neurological or psychiatric disorder is associated with dysregulated locomotor activity.

In some embodiments, the neurological or psychiatric disorder is associated with a deficit in cyclic nucleotide signaling molecules. In some embodiments, the neurological or psychiatric disorder is associated with a deficit in cAMP and/or cGMP. In some embodiments, the neurological or psychiatric disorder is associated with low activity of cAMP Responsive Element Binding Protein (CREB), Serum Response Factor (SRF), or both.

In some embodiments, the present invention provides a method of treating a neurological or psychiatric disorder described herein, comprising administering a compound of the invention in conjunction with one or more pharmaceutical agents. Suitable pharmaceutical agents that may be used in combination with the compounds of the present invention include anti-Parkinson's drugs, anti-Alzheimer's drugs, antidepressants, anti-psychotics, anti-ischemics, CNS depressants, anti-cholinergics, and nootropics.

Suitable anti-Parkinson's drugs include, but are not limited to, dopamine replacement therapy (e.g. L-DOPA, carbidopa, COMT inhibitors such as entacapone), dopamine agonists (e.g. D1 agonists, D2 agonists, mixed D1/D2 agonists; bromocriptine, pergolide, cabergoline, ropinirole, pramipexole, or apomorphine in combination with domperidone), histamine H2 antagonists, and monoamine oxidase inhibitors such as selegiline and tranylcypromine.

In some embodiments, compounds of the invention may be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include, but are not limited to, beta-secretase inhibitors, gamma-secretase inhibitors, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include, but are not limited to norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists.

Specific suitable anti-depressant and anti-anxiety agents include, but are not limited to, amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide; venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the invention relates to a method of inhibiting PDE1 in a biological sample comprising the step of contacting said biological sample with a compound of this invention, or a composition comprising said compound. In some embodiments, the PDE1 is PDE1A. In some embodiments, the PDE1 is PDE1B. In some embodiments, the PDE1 is PDE1C. In some embodiments, the invention provides a method of inhibiting PDE1B selectively over PDE1A and/or PDE1C. In some embodiments, the invention provides a method of inhibiting PDE1B selectively over PDE1A. In some embodiments, the invention provides a method of inhibiting PDE1B selectively over PDE1C. In some embodiments, the invention provides a method of inhibiting PDE1B selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. Selectivity for one PDE1 isoform over another refers to the inverse ratio of IC₅₀ values against each respective isoform as determined using the HTRF PDE1 inhibition assay described in the Examples. For example, the selectivity of a compound of this invention for PDE1B over PDE1C refers to the ratio IC₅₀(PDE1C)/IC₅₀(PDE1B), wherein IC₅₀(PDE1C) is the IC₅₀ value of the compound against PDE1C as determined using the described HTRF PDE1 inhibition assay, and IC₅₀(PDE1B) is the IC₅₀ value of the compound against PDE1B as determined using the described HTRF PDE1 inhibition assay.

In certain embodiments, the invention relates to a method of modulating cyclic nucleotide levels in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of enzymes in a biological sample is useful for a variety of purposes that are known to one of skill in the art. Examples of such purposes include, but are not limited to biological assays, gene expression studies, and biological target identification.

Another embodiment of the present invention relates to a method of inhibiting PDE1 in a patient comprising the step of administering to said patient a compound of the present invention, or a composition comprising said compound. In some embodiments, the PDE1 is PDE1B. In some embodiments, the invention provides a method of inhibiting PDE1B in a patient selectively over PDE1A and/or PDE1C. In some embodiments, the invention provides a method of inhibiting PDE1B in a patient selectively over PDE1A. In some embodiments, the invention provides a method of inhibiting PDE1B in a patient selectively over PDE1C. In some embodiments, the invention provides a method of inhibiting PDE1B in a patient selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. Selectivity for one PDE1 isoform over another refers to the inverse ratio of IC₅₀ values against each respective isoform as determined using the HTRF PDE1 inhibition assay described in the Examples. For example, the selectivity of a compound of this invention for PDE1B over PDE1C refers to the ratio IC₅₀(PDE1C)/IC₅₀(PDE1B), wherein IC₅₀(PDE1C) is the IC₅₀ value of the compound against PDE1C as determined using the described HTRF PDE1 inhibition assay, and IC₅₀(PDE1B) is the IC₅₀ value of the compound against PDE1B as determined using the described HTRF PDE1 inhibition assay.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In certain embodiments, a combination of 2 or more therapeutic agents may be administered together with compounds of the invention. In certain embodiments, a combination of 3 or more therapeutic agents may be administered with compounds of the invention.

Other examples of agents the inhibitors of this invention may also be combined with include, without limitation: vitamins and nutritional supplements, antiemetics (e.g. 5-HT₃ receptor antagonists, dopamine antagonists, NK1 receptor antagonists, histamine receptor antagonists, cannabinoids, benzodiazepines, or anticholinergics), agents for treating Multiple Sclerosis (MS) such as beta interferon (e.g., Avonex and Rebif), Copaxone, and mitoxantrone; treatments for asthma such as albuterol and Singulair; anti-inflammatory agents such as corticosteroids, TNF blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In certain embodiments, compounds of the present invention, or a pharmaceutically acceptable composition thereof, are administered in combination with antisense agents, a monoclonal or polyclonal antibody or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination”, “combined”, and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present invention provides a medicament comprising at least one compound of formula I or a pharmaceutically acceptable salt thereof and a pharmaceutically acceptable carrier.

In some embodiments, the present invention provides the use of a compound of formula I in the manufacture of a medicament for the treatment of a neurological or psychiatric disorder.

### EXAMPLES

### Exemplification

As depicted in the Examples below, in certain exemplary embodiments, compounds are prepared according to the following procedures. It will be appreciated that, although the general methods depict the synthesis of certain compounds of the present invention, the following methods, and other methods known to one of ordinary skill in the art, can be applied to all compounds and subclasses and species of each of these compounds, as described herein.

In the examples below, unless otherwise indicated, all temperatures are set forth in degrees Celsius and all parts and percentages are by weight. Reagents were purchased from commercial suppliers, such as Sigma-Aldrich Chemical Company, and were used without further purification unless otherwise indicated. Reagents were prepared following standard literature procedures known to those skilled in the art. Solvents were purchased from Aldrich in Sure-Seal bottles and used as received. All solvents requiring purification or drying were treated using standard methods known to those skilled in the art, unless otherwise indicated.

The reactions set forth below were done generally at ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aqueous KMnO₄ solution activated with heat. Flash column chromatography (See, e.g., Still et al., J. Org. Chem., 43: 2923 (1978)) was performed using silica gel 60 (Merck Art 9385) or various MPLC systems. Reactions under microwave irradiation conditions were carried out in a Biotage initiator microwave system.

The compound structures in the examples below were confirmed by one or more of the following methods: proton magnetic resonance spectroscopy, mass spectroscopy, and melting point. Proton magnetic resonance (¹H NMR) spectra were determined using a JEOL or Bruker NMR spectrometer operating at 300 or 400 MHz field strength. Chemical shifts are reported in the form of delta (6) values given in parts per million (ppm) relative to an internal standard, such as tetramethylsilane (TMS). Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; quint, quintet; sext, sextet; sept, septet; br, broadened; and m, multiplet. Coupling constants are given in Hertz (Hz). Mass spectra (MS) data were obtained using Agilent Technologies 1200 Series/Agilent Technologies 6110 Quadrupole LC/MS, Waters ACQUITY UPLC or Shimadzu LCMS-2020. Waters supercritical fluid system (SFC) was used to separate chiral compounds with the following methods.

Method A:

Column: Regis (R,R)-Whelk-O1 (4.6×250 mm, 5 μm)

Co-Solvent: MeOH (0.1% DEA)

Column Temperature: 40° C.

CO₂ Flow Rate: 1.95 mL/min

Co-Solvent Flow Rate: 1.05 mL/min

Method B:

Column: Daicel AS-H (4.6×250 mm, 5 μm)

Co-Solvent: MeOH (0.1% DEA)

Column Temperature: 40° C.

CO₂ Flow Rate: 2.25 mL/min

Co-Solvent Flow Rate: 0.75 mL/min

Method C:

Column: Daicel AS-H (4.6×250 mm, 5 μm)

Co-Solvent: MeOH (0.1% DEA)

Column Temperature: 40° C.

CO₂ Flow Rate: 2.55 mL/min

Co-Solvent Flow Rate: 0.45 mL/min

Method D:

Column: Daicel AS-H (4.6×250 mm, 5 μm)

Co-Solvent: MeOH (0.1% DEA)

Column Temperature: 40° C.

CO₂ Flow Rate: 2.4 mL/min

Co-Solvent Flow Rate: 0.6 mL/min

Method E:

Column: Daicel AS-H (4.6×250 mm, 5 μm)

Co-Solvent: MeOH/CH₃CN (1/1) (0.1% DEA)

Column Temperature: 40° C.

CO₂ Flow Rate: 2.7 mL/min

Co-Solvent Flow Rate: 0.3 mL/min

Method F:

Column: Regis (R,R)-Whelk-O1 (4.6×250 mm, 5 μm)

Co-Solvent: MeOH/CH₃CN (1/1) (0.1% DEA)

Column Temperature: 40° C.

CO₂ Flow Rate: 1.8 mL/min

Co-Solvent Flow Rate: 1.2 mL/min

Method G:

Column: IC (4.6×150 mm, 5 μm)

Co-Solvent: EtOH/n-Hexane (1/1) (0.1% DEA)

Column Temperature: 39.9° C.

CO₂ Flow Rate: 1.95 mL/min

Co-Solvent Flow Rate: 1.05 mL/min

Method H:

Column: Regis (R,R)-Whelk-O1 (4.6×250 mm, 5 μm)

Co-Solvent: MeOH (0.5% NH₄OH)

Column Temperature: 40° C.

CO₂ Flow Rate: 1.95 mL/min

Co-Solvent Flow Rate: 1.05 mL/min

As used herein, and unless otherwise specified, “Me” means methyl, “Et” means ethyl, “Ac” means acetyl, “BINAP” means 2,2′-bis(diphenylphosphino)-1,1′-binaphthyl, “Boc” means tert-butoxycarbonyl, “Dess-Martin reagent” means 1,1,1-tris(acetyloxy)-1,1-dihydro-1,2-benziodoxol-3-(1H)-one, “DCM” means dichloromethane, “DEA” means diethylamine, “DEAD” means diethyl azodicarboxylate, “DIEA” means diisopropylethylamine, “DMF” means dimethylformamide, “DME” means dimethoxyethane, “DMSO” means dimethyl sulfoxide, “EDCI” means N-(3-dimethylaminopropyl)-N′-ethylcarbodiimide hydrochloride, “EtOAc” means ethyl acetate, “EtOH” means ethanol, “HATU” means O-(7-azabenzotriazol-1-yl)-N,N,N′,N′-tetramethyluronium hexafluorophosphate, “TBTU” means O-(Benzotriazol-1-yl)-N,N,N′,N′-tetramethyluronium tetrafluoroborate, “HOBt” means hydroxybenzotriazole, “NBS” means 1-bromopyrrolidine-2,5-dione, “NCS” means N-Chlorosuccinimide, “NIS” means N-iodosuccinimide, “NNP” means N-methylpyrrolidone, “m-CPBA” means 3-chloro-perbenzoic acid, “MeCN” means acetonitrile, “MeOH” means methanol, “NMO” means N-methyl morpholine N-oxide, “PE” means petroleum ether, “RT” or “rt” means room temperature, “t-BuOH” means tert-butanol, “t-BuONa” means sodium tert-butoxide, “TBDMSCl” means tert-butyldimethylsilyl chloride, “TEA” means triethylamine, “THF” means tetrahydrofuran, “TMSI” means iodotrimethylsilane, “Xantphos” means 4,5-bis(diphenylphosphino)-9,9-dimethylxanthene, “X-phos” means (2′,4′,6′-Triisopropylbiphenyl-2-yl)phosphine, “h” or “hr” means hour(s), “rt” means room temperature, “min” means minute(s), “cat.” means catalytic, “aq” means aqueous, “TMSI” means trimethylsilyl iodide, “TFA” means trifluoroacetic acid, “TFAA” means trifluoroacetic anhydride, “TsCl” means tosyl chloride and “MsCl” means methanesulfonyl chloride.

### Reference Example 1

### 8-(Propan-2-yl)-2,3-dihydro-1H,5H-diimidazo[2,1-c:5′,1′-f][1,2,4]triazin-5-one

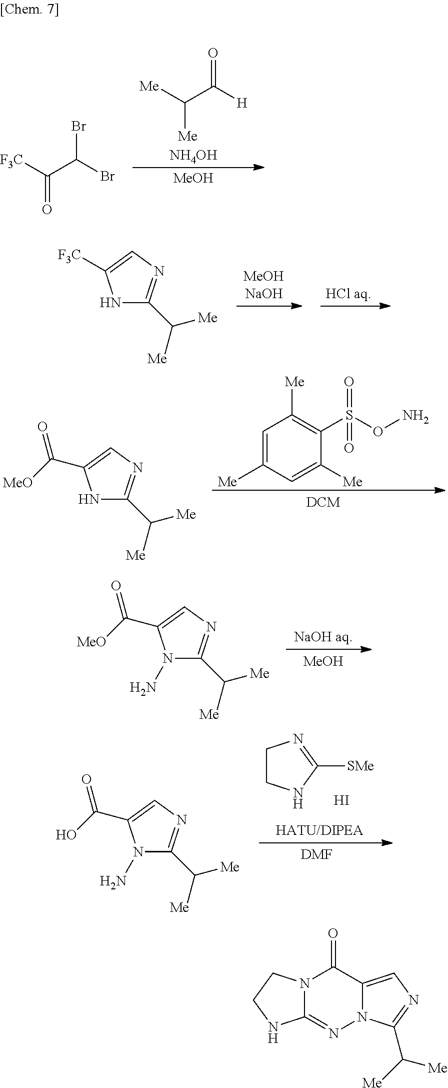

To a solution of 2-(methylsulfanyl)-4,5-dihydro-1H-imidazole hydroiodide (464 mg, 4 mmol) in DMF (20 mL) was added 1-amino-2-(propan-2-yl)-1H-imidazole-5-carboxylic acid (676 mg, 4 mmol), HATU (3 g, 8 mmol) and DIPEA (1.56 g, 12 mmol). The reaction mixture was stirred at r.t. overnight. Then the mixture was extracted by DCM from water. The organic layer was collected and concentrated to get crude product. The title compound was purified by reversed phase (0.05% NH₃ in Water and MeCN) as white solid (300 mg, 34%). LCMS (m/z)=220 [M+H]⁺. H NMR (400 MHz, CDCl₃): δ 1.36 (d, J=7.2 Hz, 6H), 3.31-3.38 (m, 1H) 3.80 (t, J=8.0 Hz, 2H), 4.19 (t, J=8.0 Hz, 2H), 4.64 (s, 1H), 7.75 (s, 1H).

### 2-(Propan-2-yl)-5-(trifluoromethyl)-1H-imidazole

A mixture of 3,3-dibromo-1,1,1-trifluoropropan-2-one (180 g, 670 mmol) and sodium acetate trihydrate (110.2 g, 1346 mmol) in water (360 mL) was heated to reflux for 1 hour. Then the mixture was cooled to room temperature. After cooling, the mixture was slowly added to a solution of isobutyraldehyde (48.29 g, 670 mmol) and Ammonium Hydroxide (725 mL) in methanol (1500 mL). The mixture was stirred at room temperature overnight. Upon the completion, methanol was removed in vacuo and the aqueous phase was extracted with ethyl acetate (800 mL×3), dried over sodium sulfate and concentrated in vacuo. The crude product (109.9 g, 92%) was obtained and used directly for next step without further purification. LC-MS (m/z)=179 [M+H]⁺.

### Methyl 2-(propan-2-yl)-1H-imidazole-5-carboxylate

A mixture of 2-(propan-2-yl)-5-(trifluoromethyl)-1H-imidazole (109.89 g, 620 mmol) and sodium hydroxide (74 g, 1850 mmol) in a cosolvent of water/methanol (664 mL/885 mL) were stirred at room temperature overnight. Then the pH was adjusted to 3-4 with HCl. The solution was stirred at room temperature for 4 hours. Upon the completion, methanol was removed and potassium carbonate was added to adjust the pH to 8-9, and the aqueous phase was extracted with ethyl acetate (800 mL×3), dried over sodium sulfate and concentrated in vacuo to give the crude product as a yellow solid (87.5 g, 84%), which was used directly for next step. LC-MS (m/z)=169 [M+H]⁺.

### Methyl 1-amino-2-(propan-2-yl)-1H-imidazole-5-carboxylate

To a mixture of methyl 2-(propan-2-yl)-1H-imidazole-5-carboxylate (50 g, 300 mmol) in dichloromethane (600 mL) was added O-(mesitylsulfonyl)hydroxylamine (160 g, 740 mmol) at 0° C. After stirring for 2 hours, potassium carbonate (235 g, 740 mmol) was added at 0° C., followed by stirring at 0° C. overnight. Upon the completion, the mixture was filtered and the filtrate concentrated in vacuo, purified by silica gel chromatography (eluted with petroleum ether/ethyl acetate=5/1) to give the title compound (40 g, 73%) as a colorless oil. LC-MS (m/z)=184 [M+H]⁺. ¹H NMR (400 MHz, DMSO-d₆): δ 1.18-1.19 (m, 6H), 3.25-3.28 (m, 1H), 3.76 (s, 3H), 5.97 (s, 2H), 7.45 (s, 1H).

### 1-Amino-2-(propan-2-yl)-1H-imidazole-5-carboxylic acid

A mixture of methyl 1-amino-2-(propan-2-yl)-1H-imidazole-5-carboxylate (183 mg, 1 mmol) and NaOH aq. (2 N, 1.5 mL) in MeOH (2 mL) was stirred at room temperature for 3 h. 6 N HCl aq. was added dropwise to adjust the pH to 4-5. The mixture was filtrated, washed with water to give crude compound (160 mg, 0.9 mmol, 90%) as white solid. LC-MS (m/z)=170 [M+H]⁺.

The compounds of Reference Example 2 to 5 were synthesized in a similar manner to Reference Example 1.

The compounds of Reference Examples 2 to 5 were synthesized in a similar manner to Reference Example 1.

TABLE 1

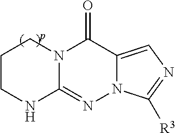

| No. | p | R³— | ¹H NMR |
|---|---|---|---|
| 2 | 0 | 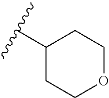 [SMILES: *C1CCOCC1] | (400 MHz, CDCl₃): δ 1.77-1.86 (m, 4H), 3.20-3.27 (m, 1H), 3.41-3.47 (m, 2H), 3.61 (t, J = 8.0 Hz, 2H), 3.91-4.02 (m, 4H), 7.54 (s, 1H), 7.78 (s, 1H) |
| 3 | 1 | 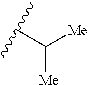 [SMILES: *C(C)C] | (400 MHz, DMSO-d₆): δ 1.25 (d, J = 6.8 Hz, 6H), 1.90-1.96 (m, 2H), 3.22-3.30 (m, 3H), 3.84 (t, J = 6.0 Hz, 2H), 7.50-7.51 (m, 2H). |
| 4 | 1 | 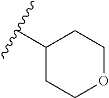 [SMILES: *C1CCOCC1] | (400 MHz, CDCl₃): δ 1.77-1.94 (m, 4H), 3.21-3.25 (m, 4H), 3.26-3.28 (m, 1H), 3.40-3.46 (m, 2H), 3.85 (t, J = 5.6 Hz, 2H), 3.91-3.95 (m, 2H), 7.54 (s, 2H). |
| 5 | 2 | 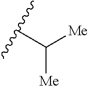 [SMILES: *C(C)C] | (400 MHz, CDCl₃): δ 1.37 (d, J = 6.8 Hz, 6H), 1.84 (s, 4H), 3.23 (s, 2H), 3.36-3.43 (m, 1H), 4.20 (s, 2H), 4.44 (s, 1H), 7.78 (s, 1H). |

### Reference Example 6

### 3-Methyl-9-(propan-2-yl)-1,2,3,4-tetrahydro-6H-imidazo[5,1-f]pyrimido[2,1-c][1,2,4]triazin-6-one

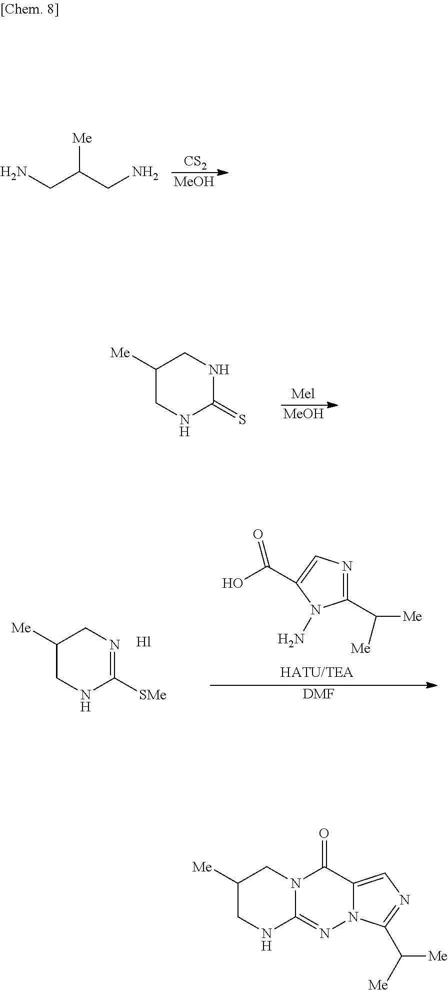

To a solution of 1-amino-2-(propan-2-yl)-1H-imidazole-5-carboxylic acid (200 mg, 1.18 mmol) in DMF (10 mL) was added 5-methyl-2-(methylsulfanyl)-1,4,5,6-tetrahydropyrimidine hydroiodide (170 mg, 1.18 mmol), HATU (674.6 mg, 1.78 mmol) and TEA (179.3 mg, 1.78 mmol). The mixture was stirred at room temperature overnight. The title compound was purified by reversed phase (0.01% NH₃ in Water and MeCN) as white solid. LC-MS (m/z)=248 [M+H]⁺. ¹H NMR (400 MHz, CDCl₃): δ 1.14 (d, J=6.4 Hz, 3H), 1.35 (d, J=7.2 Hz, 6H), 2.16-2.26 (m, 1H), 3.02-3.08 (m, 1H), 3.18-3.24 (m, 1H), 3.31-3.38 (m, 1H), 3.44-3.48 (m, 1H), 4.41-4.46 (m, 1H), 4.94 (s, 1H), 7.75 (s, 1H).

### 5-Methyltetrahydropyrimidine-2(1H)-thione

To a solution of 2-methylpropane-1,3-diamine (3.2 g, 36.4 mmol) in MeOH (15 mL) was added CS₂ (2.76 g, 36.4 mmol). The mixture was refluxed overnight. The title compound was purified by column chromatography (EtOAc/PE=1/2) as oil. LC-MS (m/z)=131 [M+H]⁺. ¹H NMR (400 MHz, CDCl₃): δ 1.04 (d, J=6.8 Hz, 3H), 2.13 (s, 1H), 2.89-2.97 (m, 2H), 3.30-3.35 (m, 2H), 6.58 (s, 2H).

### 5-Methyl-2-(methylsulfanyl)-1,4,5,6-tetrahydropyrimidine hydroiodide

To a solution of 5-methyltetrahydropyrimidine-2(1H)-thione (800 mg, 6.16 mmol) in MeOH (10 mL) was added MeI (1.05 g, 7.38 mmol). The mixture was refluxed overnight. The solvents were removed under reduced pressure to give crude product (1.67 g, 6.1 mmol, 97%) as yellow solid. LC-MS (m/z)=145 [M+H]⁺. ¹H NMR (400 MHz, CDCl₃): δ 1.10 (d, J=4.0 Hz, 3H), 2.10-2.19 (m, 1H), 2.85 (s, 3H), 3.04-3.10 (m, 2H), 3.26 (s, 1H), 3.71-3.74 (m, 2H).

### Reference Example 7

### 2-Methyl-9-(propan-2-yl)-1,2,3,4-tetrahydro-6H-imidazo[5,1-f]pyrimido[2,1-c][1,2,4]triazin-6-one

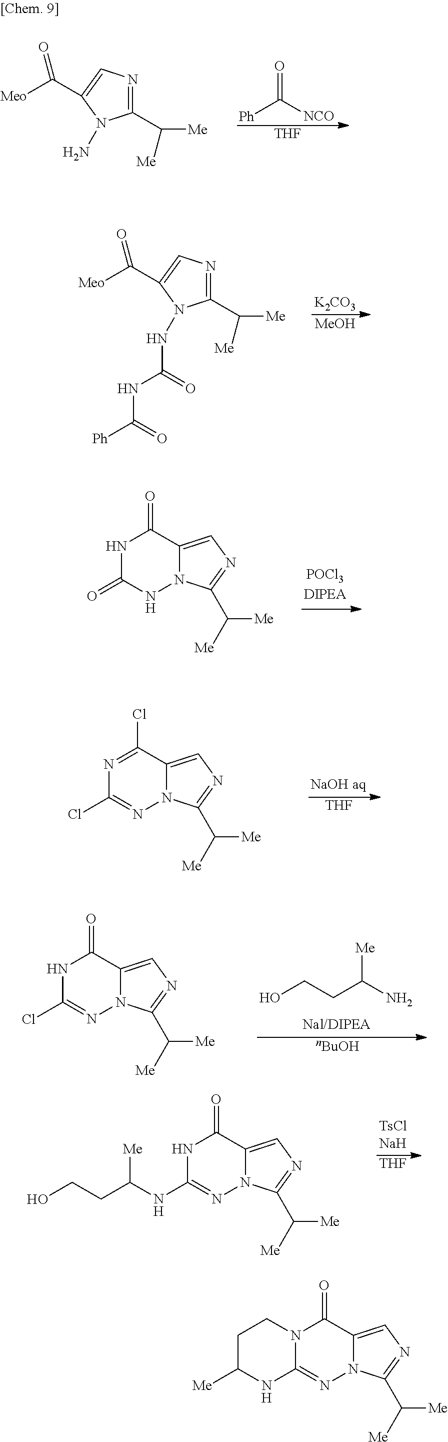

A solution of 2-[(4-Hydroxybutan-2-yl)amino]-7-(propan-2-yl)imidazo[5, 1-f][1,2,4]triazin-4(3H)-one (150 mg, 0.57 mmol) in 10 mL anhydrous THF was added NaH (60% in oil, 113 mg, 2.8 mmol) at 0° C. under N₂ protection. The mixture was stirred at 0° C. for TsCl (107 mg, 0.57 mmol) was dropped into the mixture under N₂ protection. The mixture was stirred at 25° C. for 1 h, and then quenched by adding 1 mL aq. NH₄Cl. The product was purified through flash-column to give the title compound (76 mg, 55%). LC-MS (m/z)=248 [M+H]⁺. ¹H NMR (400 MHz, CD₃OD): δ 1.11-1.17 (m, 3H), 1.20-1.22 (m, 6H), 1.52-1.61 (m, 1H), 2.02-2.09 (m, 1H), 3.29-3.36 (m, 1H), 3.48-3.55 (m, 2H), 4.20-4.25 (m, 1H), 7.47 (s, 1H).

### Methyl 1-[(benzylcarbamoyl)amino]-2-(propan-2-yl)-1H-imidazole-5-carboxylate

A solution of methyl 1-amino-2-(propan-2-yl)-1H-imidazole-5-carboxylate (30 g, 164 mmol), benzoyl isocyanate (29 g, 197 mmol) in THF (900 ml) was stirred at 75° C. for 2 h. Then it was concentrated in vacuo to give the title compound as a white solid (50.7 g, 94%). LC-MS (m/z)=331 [M+H]⁺.

### 7-(Propan-2-yl)imidazo[5,1-f][1,2,4]triazine-2,4(1H,3H)-dione

To a solution of methyl 1-[(benzoylcarbamoyl)amino]-2-(propan-2-yl)-1H-imidazole-5-carboxylate (48.8 g, 148 mmol) in methanol (1 L) was added potassium carbonate (40.8 g, 296 mmol). The reaction mixture was stirred at 60° C. for 5 h. Then it was filtered and the filtrate was concentrated and HCl was added until pH=5-6. White solid precipitated to give the title compound as a white solid (23.5 g, 82%). LC-MS (m/z)=195 [M+H]⁺. ¹H NMR (400 MHz, DMSO-d₆): δ 1.25-1.26 (m, 6H), 7.52-7.97 (m, 2H), 11.13 (br, 1H), 13.30 (br, 1H).

### 2,4-Dichloro-7-(propan-2-yl)imidazo[5,1-f][1,2,4]triazine

To a solution of 7-(Propan-2-yl)imidazo[5, 1-f][1,2,4]triazine-2,4(1H,3H)-dione (10 g, 51.5 mmol) in phosphorus oxychloride (70 mL) was added N,N-diisopropylethylamine (8 g, 61.9 mmol). The reaction mixture was stirred at 120° C. for 3 h. Then it was concentrated used directly for next step without further purification. LC-MS (m/z)=231 [M+H]⁺.

### 2-Chloro-7-(propan-2-yl)imidazo[5,1-f][1,2,4]triazin-4(3H)-one

To the reaction mixture (2,4-dichloro-7-(propan-2-yl)imidazo[5,1-f][1,2,4]triazine) was added 2 N NaOH (200 mL, 0.6 mol) and THF (300 mL). The mixture was stirred at 50° C. for 3 h. Then it was concentrated and HCl was added until pH=5-6. It was extracted with dichloromethane (100 mL×3). The combined organics were washed with brine (50 ml×2), dried over sodium sulfate, concentrated and purified by silica gel (eluted with DCM/methanol=20/1) to give the title compound as a light white solid (8.0 g, 73% for 2 steps). LC-MS (m/z)=213 [M+H]⁺. ¹H NMR (400 MHz, DMSO-d₆): δ 1.26-1.27 (m, 6H), 3.31-3.38 (m, 1H), 7.72 (s, 1H), 12.99 (br, 1H).

### 2-[(4-Hydroxybutan-2-yl)amino]-7-(propan-2-yl)imidazo[5,1-f][1,2,4]triazin-4(3H)-one

A solution of 2-chloro-7-(propan-2-yl)imidazo[5, 1-f][1,2,4]triazin-4(3H)-one (300 mg, 1.41 mmol), 3-aminobutan-1-ol (378 mg, 4.24 mmol), DIPEA (1.8 g, 14.1 mmol) and NaI (212.2 mg, 1.41 mmol) in 6 mL n-BuOH was heated to 170° C. for 16 h under N₂ protection. The reaction was cooled to 25° C., and then purified through flash-column to give the title compound (153 mg, 40%). LC-MS (m/z)=266 [M+H]⁺. ¹H NMR (400 MHz, CDCl₃): δ 1.34-1.36 (m, 3H), 1.41-1.44 (m, 9H), 1.73-1.80 (m, 1H), 1.84-1.93 (m, 1H), 3.47-3.52 (m, 1H), 3.69-3.74 (m, 1H), 3.77-3.81 (m, 1H), 4.11-4.16 (m, 1H), 7.44 (s, 1H).

### Reference Example 8

### (R)-2-(4-Chlorophenyl)-9-(tetrahydro-2H-pyran-4-yl)-1,2,3,4-tetrahydro-6H-imidazo[5,1-f]pyrimido[2,1-c][1,2,4]triazin-6-one

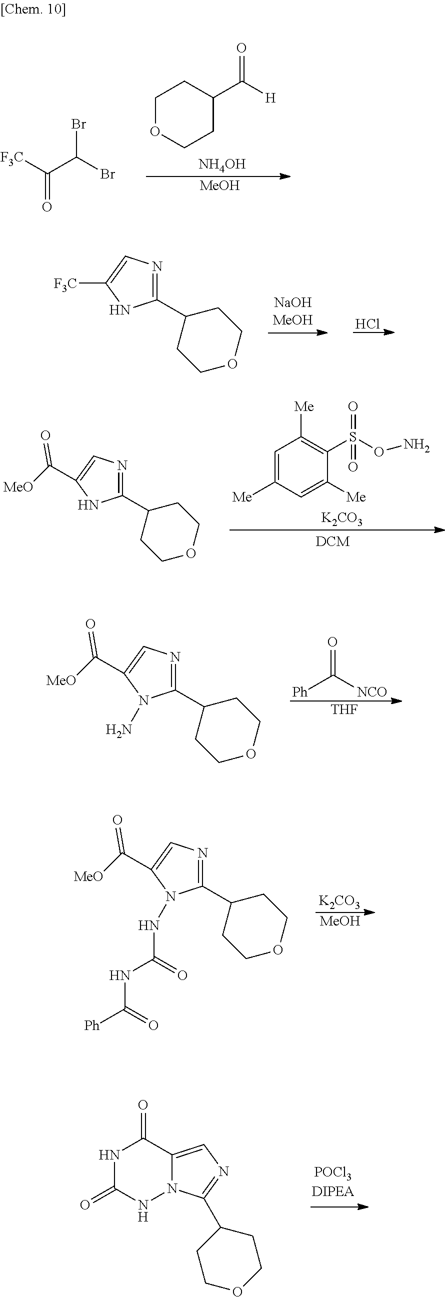

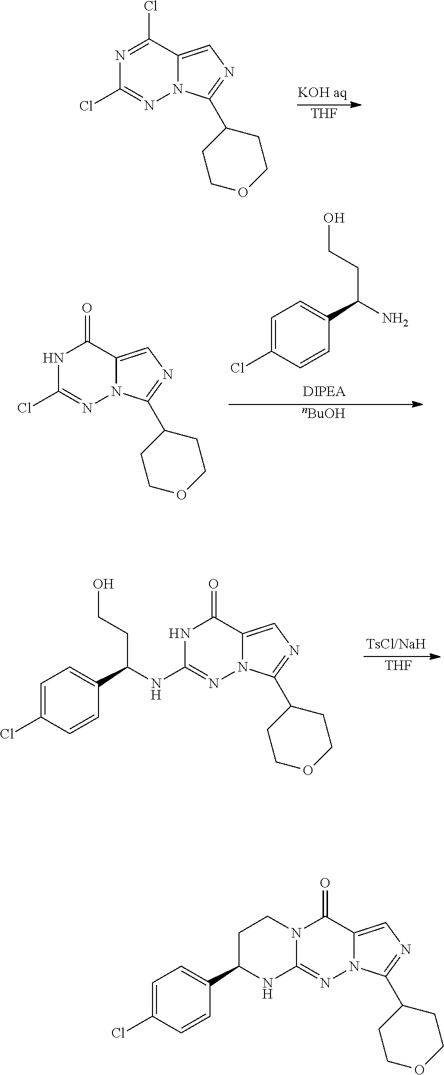

To a solution of (R)-2-((1-(4-chlorophenyl)-3-hydroxypropyl)amino)-7-(tetrahydro-2H-pyran-4-yl)imidazo[5,1-f][1,2,4]triazin-4(3H)-one (600 mg, 1.49 mmol) in THF (10 mL) was added sodium hydride (0.11 g, 4.47 mmol) and TsCl (0.34 g, 1.79 mmol). The reaction mixture was stirred for 30 min, and then purified by prep-HPLC to give the title compound (282 mg, 50%) as white solid. LC-MS (m/z)=386 [M+H]⁺. ¹H NMR (CDCl₃, 400 MHz) δ: 7.79 (s, 1H), 7.41 (d, J=8.4 Hz, 2H), 7.32 (d, J=8.4 Hz, 2H), 5.12 (s, 1H), 4.70-4.67 (m, 1H), 4.26-4.20 (m, 1H), 4.09-4.07 (m, 2H), 3.87-3.80 (m, 1H), 3.60-3.53 (m, 2H), 3.35-3.27 (m, 1H), 2.40-2.34 (m, 1H), 2.15-2.01 (m, 3H), 1.69-1.67 (m, 2H).

### 2-(Tetrahydro-2H-pyran-4-yl)-5-(trifluoromethyl)-4,5-dihydro-1H-imidazole

A mixture of sodium acetate trihydrate (27.2 g, 200 mmol) and 3, 3-dibromo-1, 1, 1-trifluoropropan-2-one (26.98 g, 100 mmol) in water (75 ml) was heated under reflux for 1 h. The mixture was then cooled to r.t. and was slowly added to a solution of tetrahydro-2H-pyran-4-carbaldehyde (90 mmol, 10.27 g) and concentrated ammonium hydroxide solution (50 mL) in MeOH (150 mL). The mixture was stirred at r.t. for 18 h and was then evaporated under reduced pressure. The aqueous residue was extracted with EtOAc (150 mL×3) and the combined organic solution was dried over magnesium sulfate and concentrated in vacuo to give an oil. The oil was then triturated in water with a trace of MeOH to afford the title compound as a crystalline solid in 90% yield (19.8 g). LC-MS (m/z)=221 [M+H]⁺.

### Methyl 2-(tetrahydro-2H-pyran-4-yl)-4,5-dihydro-1H-imidazole-5-carboxylate

To a solution of methyl 2-(tetrahydro-2H-pyran-4-yl)-5-(trifluoromethyl)-4,5-dihydro-1H-imidazole (85 mmol) in MeOH (200 mL) was added NaOH solution (2.7 M, 50 mL) and the mixture was stirred at 95 C overnight. Then conc. HCl (25 mL) was added. The mixture was stirred at that temperature for 4 h. EtOAc (250 mL) was added to the reaction vessel and the resulting biphasic mixture was transferred to a separatory funnel. The layers were separated and the water phase was extracted with EtOAc (150 mL×3). The combined organics were dried over anhydrous Na₂SO₄, filtered and concentrated in vacuum to afford the title compound as a solid in 80% yield (16.5 g). LC-MS (m/z)=210 [M+H]⁺

### Amino-2-(tetrahydro-2H-pyran-4-yl)-4,5-dihydro-1H-imidazole-5-carboxylate

To a solution of 2-(tetrahydro-2H-pyran-4-yl)-4, 5-dihydro-1H-imidazole-5-carboxylate (70 g, 0.34 mol) in DCM (250 mL) was added O-(mesitylsulfonyl) hydroxylamine (110 g, 0.51 mol) and K₂CO₃ (94 g, 0.64 mol). The reaction mixture was cooled to 0° C. and stirred at that temperature for 15 h. Water (50 mL) was added to the reaction vessel and the resulting biphasic mixture was transferred to a separatory funnel. The layers were separated. The combined organics were dried over anhydrous Na₂SO₄, filtered and concentrated in vacuum. The resulting solid was purified by flash column chromatography to provide the title compound (25 g, 35%) as a white solid. LC-MS (m/z)=225 [M+H]+

### Methyl 1-(3-benzoylureido)-2-(tetrahydro-2H-pyran-4-yl)-1H-imidazole-5-carboxylate

To a solution of methyl 1-amino-2-(tetrahydro-2H-pyran-4-yl)-1H-imidazole-5-carboxylate (5 g, 22.2 mmol) in THF (75 mL) was added benzoyl isocyanate (3.59 g, 24.42 mmol). The reaction mixture was heated to and stirred at that temperature for 12 h. The combined organics was concentrated in vacuo to give the title compound (5 g, 80%). LC-MS (m/z)=373 [M+H]⁺. ¹H NMR (400 MHz, DMSO-d₆): δ 1.67-1.88 (m, 4H), 3.04-3.12 (m, 1H), 3.40-3.46 (m, 2H), 3.71 (s, 3H), 3.89-3.94 (m, 2H), 7.56-7.60 (m, 2H), 7.67-7.71 (m, 2H), 8.06-8.08 (m, 2H), 11.19 (s, 1H), 11.34 (s, 1H).

### 7-(Tetrahydro-2H-pyran-4-yl)imidazo[5,1-f][1,2,4]triazine-2,4(1H,3H)-dione

To a solution of methyl 1-(3-benzoylureido)-2-(tetrahydro-2H-pyran-4-yl)-1H-imidazole-5-carboxylate (5 g, 13.43 mmol) in methanol (45 mL) was added potassium carbonate (2.23 g, 16.11 mmol). The combined organics concentrated in vacuo. Water (20 mL) was added to the reaction. The mixture was neutralized with 1 N HCl, filtered and washed with MeOH to give the title compound (1.8 g, 56%). LC-MS (m/z)=237 [M+H]⁺. ¹H NMR (400 MHz, DMSO-d₆): δ 1.75-1.85 (m, 4H), 3.32-3.35 (m, 1H), 3.38-3.49 (m, 2H), 3.92-3.95 (m, 2H), 7.50 (br, 1H), 7.74 (s, 1H), 11.15 (s, 1H).

### 2,4-Dichloro-7-(tetrahydro-2H-pyran-4-yl)imidazo[5,1-f][1,2,4]triazine

To the mixture of 7-(tetrahydro-2H-pyran-4-yl)imidazo[5,1-f][1,2,4]triazine-2,4(1H,3H)-dione (1.8 g) in phosphoryl trichloride (20 mL), N,N-diisopropylethylamine (1.48 g) was added. The mixture was stirred for 3 h at 120° C. The pH was adjusted to 7-8 and a white precipitate formed. After filtration, the title compound was collected (1.2 g, 60%) as a yellow solid. LC-MS (m/z)=273 [M+H]⁺. ¹H NMR (400 MHz, DMSO-d₆): δ 1.24-1.31 (m, 2H), 1.82-1.87 (m, 2H), 3.40-3.54 (m, 1H), 3.47-3.54 (m, 2H), 3.92-3.96 (m, 2H), 7.88 (s, 1H).

### 2-Chloro-7-(tetrahydro-2H-pyran-4-yl)imidazo[5,1-f][1,2,4]triazin-4(3H)-one

To the solution of 2,4-dichloro-7-(tetrahydro-2H-pyran-4-yl)imidazo[5, 1-f][1,2,4]triazine (1.2 g) in tetrahydrofuran (20 mL) was added 2N KOH (20 mL). The mixture was stirred for 2 h at 50° C., and then was neutralized with 1N HCl. The mixture was filtered to get the product (0.78 g, 70%). LC-MS (m/z)=255 [M+H]⁺. ¹H NMR (400 MHz, DMSO-d₆): δ 1.79-1.88 (m, 4H), 3.34-3.38 (m, 1H), 3.46-3.53 (m, 2H), 3.91-3.95 (m, 2H), 7.76 (s, 1H), 13.01 (br, 1H).

### (R)-2-((1-(4-chlorophenyl)-3-hydroxypropyl)amino-7-(tetrahydro-2H-pyran-4-yl)imidazo[5,1-f][1,2,4]triazin-4(3H)-one

(R)-3-amino-3-(4-chlorophenyl)propan-1-ol (370 mg, 2 mmol) and 2-chloro-7-(tetrahydro-2H-pyran-4-yl)imidazo[5,1-f][1,2,4]triazin-4(3H)-one (508 mg, 1 mmol), N,N-diisopropylethylamine (0.78 g, 6 mmol) in butyl alcohol (6 mL) was heated to 170° C. for 15 h under microwave. The mixture was purified by column chromatography (DCM/MeOH=20/1) to give the title compound (600 mg, 48%) as a colorless oil. LC-MS (m/z)=404 [M+H]⁺

The compounds of Reference Examples 9 and 10 were synthesized in a similar manner to Reference Example 8.

TABLE 2

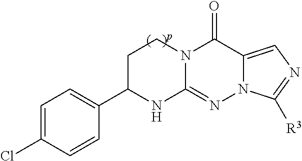

| No. | p | R³ | ¹H NMR |
|---|---|---|---|
| 9 | 0 | 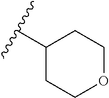 [SMILES: *C1CCOCC1] | (400 MHz, CDCl₃): δ 7.80 (s, 1H), 7.45- 7.43 (m, 2H), 7.39 (t, J = 11.2 Hz, 2H), 5.12 (t, J = 8.2 Hz, 1H), 4.59 (t, J = 10.0 Hz, 1H), 4.13-4.09 (m, 2H), 3.93-3.89 (m, 1H), 3.63-3.57 (m, 1H), 3.37-3.33 (m, 2H), 3.31 (s, 1H), 2.13-2.07 (m, 2H), 1.95-1.91 (m, 2H). |
| 10 | 1 | 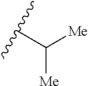 [SMILES: *C(C)C] | (400 MHz, CDCl₃): δ 7.82 (s, 1H), 7.42 (d, J = 8.0 Hz, 2H), 7.33 (d, J = 8.4 Hz, 2H), 5.07 (hr, 1H), 4.71-4.68 (m, 1H), 4.32-4.26 (m, 1H), 4.12-4.09 (m, 2H), 3.89-3.82 (m, 1H), 3.58 (t, J = 12.0 Hz, 2H), 3.37-3.32 (m, 1H), 2.41-2.37 (m, 1H), 2.15-2.03 (m, 3H), 1.60 (hr, 2H). |

### Reference Example 11

### (S)-2-(4-methoxyphenyl)-8-(tetrahydro-2H-pyran-4-yl)-2,10-dihydro-3H,5H-diimidazo[2,1-c:5′,1′-f][1,2,4]triazin-5-one

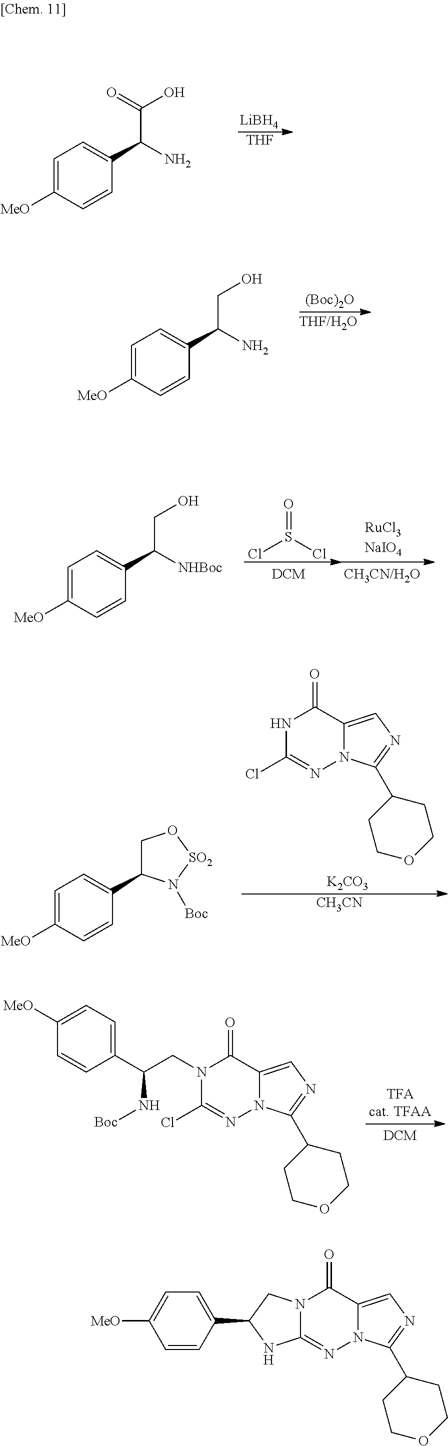

A mixture of 2-methyl-2-propanyl [(1S)-2-[2-chloro-4-oxo-7-(tetrahydro-2H-pyran-4-yl)imidazo[5, 1-f][1,2,4]triazin-3(4H)-yl]-1-(4-methoxyphenyl)ethyl]carbamate (1.26 g, 2.50 mmol), trifluoroacetic acid (1.90 mL, 25.0 mmol), and trifluoroacetic anhydride (17.0 μL, 0.125 mL) in dichloromethane (8.30 mL) was stirred at rt for 4 h. The resulting mixture was concentrated in vacuo. To an ice-cooled mixture of the above residue in THF (8.30 mL) was added triethylamine (3.50 mL, 25.0 mmol), and the reaction mixture was stirred at rt overnight. The resulting mixture was diluted with water, and extracted with ethylacetate. The combined organic extracts were dried over sodium sulfate, filtered, and concentrated in vacuo. The solid was washed with ethylacetate to give the title compound (666 mg). ¹H NMR (400 MHz, CDCl₃): δ 7.74 (s, 1H), 7.29-7.26 (m, 2H), 6.93-6.90 (m, 2H), 5.17 (s, 1H), 5.04 (t, J=8.0 Hz, 1H), 4.48 (dd, J=11.3, 8.7 Hz, 1H), 4.08-4.02 (m, 2H), 3.90-3.85 (m, 1H), 3.80 (s, 3H), 3.58-3.52 (m, 2H), 3.33-3.25 (m, 1H), 2.11-1.98 (m, 2H), 1.91-1.85 (m, 2H).

### (2S)-2-Amino-2-(4-methoxyphenyl)ethanol

To an ice-cooled mixture of lithium borohydride (16.5 g, 759 mmol) in THF (270 mL) was added trimethylsilyl chloride (194 mL, 1.52 mol). After stirring for 30 minutes, a mixture of (2S)-2-amino-2-(4-methoxyphenyl)acetic acid (45.9 g, 253 mmol) in THF (1.00 L) was added dropwise to the reaction. Then the reaction mixture was stirred at rt overnight. The reaction was quenched with methanol, and the resulting mixture was concentrated in vacuo. The residue was diluted with 1 M aqueous sodium hydroxide solution and chloroform, and filtered through a pad of Celite. The filtrate was diluted with brine, and organic layer was separated. The aqueous layer was extracted with chloroform. The combined organic extracts were dried over sodium sulfate, filtered, and concentrated in vacuo to give the title compound (37.1 g) as a crude product. ¹H NMR (400 MHz, CDCl₃): δ 7.26-7.24 (m, 3H), 6.89 (d, J=8.3 Hz, 2H), 4.02-3.99 (m, 1H), 3.80 (s, 3H), 3.73-3.69 (m, 1H), 3.55-3.50 (m, 1H), 1.72 (br s, 3H).

### 2-Methyl-2-propanyl [(1S)-2-hydroxy-1-(4-methoxyphenyl)ethyl]carbamate

A mixture of (2S)-2-amino-2-(4-methoxyphenyl)ethanol (37.1 g, 222 mmol), (Boc)₂ O (50.8 g, 233 mmol), sodium carbonate (24.7 g, 233 mmol) in THF-water (750 mL, 2:1) was stirred at rt overnight. The resulting mixture was filtered through a pad of Celite, and the cake was washed with ethyl acetate. The filtrate was extracted with ethyl acetate. The combined organic extracts were dried over sodium sulfate, filtered, and concentrated in vacuo to give the title compound (59.0 g) as a crude product. ¹H NMR (400 MHz, CDCl₃): δ 7.24-7.21 (m, 2H), 6.92-6.88 (m, 2H), 5.12 (br s, 1H), 4.72 (br s, 1H), 3.84-3.80 (m, 5H), 2.34 (br s, 1H), 1.43 (s, 9H).

### 2-Methyl-2-propanoyl (4S)-4-(4-methoxyphenyl)-1,2,3-oxathiazolidine-3-carboxylate 2,2-dioxide

To a mixture of 2-methyl-2-propanyl [(1S)-2-hydroxy-1-(4-methoxyphenyl)ethyl]carbamate (5.00 g, 18.7 mmol) and triethylamine (7.80 mL, 56.1 mmol) in dichloromethane (171 mL) was added the mixture of thionyl chloride (1.60 mL, 22.1 mmol) in dichloromethane (19.0 mL) at −40° C., and the reaction mixture was stirred at −40° C. for 2 h. The reaction was quenched with water, and extracted with chloroform. The combined organic extracts were dried over sodium sulfate, filtered, and concentrated in vacuo. To an ice-cooled mixture of the above residue and ruthenium chloride n-hydrate (39.0 mg, 0.187 mmol) in acetonitrile/water (90.0 mL, 2/1) was added sodium periodate (6.00 g, 28.1 mmol) stirred at rt for 3 h. The resulting mixture was concentrated in vacuo. The residue was diluted with water and extracted with ethylacetate. The combined organic extracts were washed with brine, dried over sodium sulfate, filtered, and concentrated in vacuo. The solid was washed with chloroform, diisopropylether, and methanol to give the title compound (2.43 g). ¹H NMR (400 MHz, CDCl₃): δ 7.36-7.33 (m, 2H), 6.95-6.91 (m, 2H), 5.25-5.23 (m, 1H), 4.86-4.82 (m, 1H), 4.41-4.38 (m, 1H), 3.82 (s, 3H), 1.44 (s, 9H).

### 2-Methyl-2-propanyl

### [(1S)-2-[2-chloro-4-oxo-7-(tetrahydro-2H-pyran-4-yl)imidazo[5,1-f][1,2,4]triazin-3(4H)-yl]-1-(4-methoxyphenyl)ethyl]carbamate

A mixture of 2-methyl-2-propanyl (4S)-4-(4-methoxyphenyl)-1,2,3-oxathiazolidine-3-carboxylate 2,2-dioxide (24.3 g, 73.6 mmol), 2-chloro-7-(tetrahydro-2H-pyran-4-yl)imidazo[5,1-f][1,2,4]triazin-4(3H)-one (17.0 g, 66.9 mmol), and potassium carbonate (10.2 g, 73.6 mmol) in acetonitrile (335 mL) was stirred at 50° C. overnight. The reaction was quenched with 1M hydrochloric acid. After stirring for 1 h at rt, saturated aqueous sodium bicarbonate solution was added to the ice-cooled mixture. The precipitate was collected. The solid was purified by silica gel chromatography (hexane/ethylacetate) to give the title compound (13.1 g). ¹H-NMR (400 MHz, CDCl₃): δ 7.87 (s, 1H), 7.32-7.30 (m, 2H), 6.95-6.92 (m, 2H), 5.20-5.14 (m, 1H), 5.03 (d, J=8.5 Hz, 1H), 4.81-4.74 (m, 1H), 4.13-4.08 (m, 3H), 3.82 (s, 3H), 3.64-3.57 (m, 2H), 3.48-3.40 (m, 1H), 2.15-2.00 (m, 2H), 1.93-1.87 (m, 2H), 1.16 (s, 9H).

### Reference Example 12

### 2-(4-Methoxyphenyl)-8-(tetrahydro-2H-pyran-4-yl)-2,10-dihydro-3H,5H-diimidazo[2,1-c:5′,1′-f][1,2,4]triazin-5-one

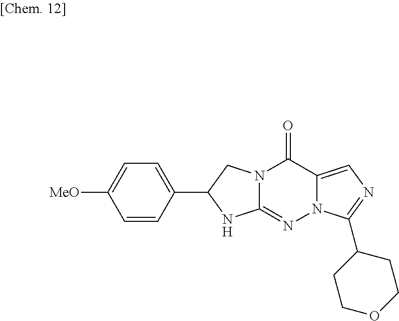 [SMILES: COc1ccc(C2Cn3c(nn4c(C5CCOCC5)ncc4c3=O)N2)cc1]

The titled compound was synthesized in a similar manner to Reference Example 11.

¹H NMR (400 MHz, CDCl₃): δ 7.79 (s, 1H), 7.33-7.28 (m, 2H), 6.97-6.95 (m, 2H), 5.08-5.02 (m, 2H), 4.56-4.51 (m, 1H), 4.12-4.09 (m, 2H), 3.95-3.90 (m, 1H), 3.83 (s, 3H), 3.63-3.56 (m, 2H), 3.37-3.31 (m, 1H), 2.13-2.07 (m, 2H), 1.94-1.91 (m, 2H).

### Reference Example 13

### 2-(5-Chloropyridin-2-yl)-8-isopropyl-2,3-dihydrodiimidazo[2,1-c:1′,5′-f][1,2,4]triazin-5(1H)-one

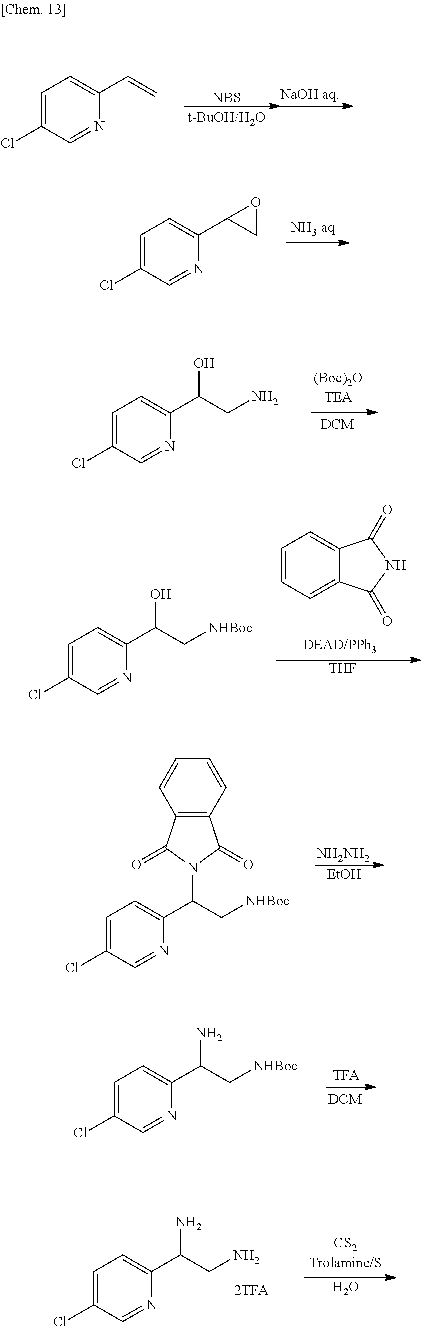

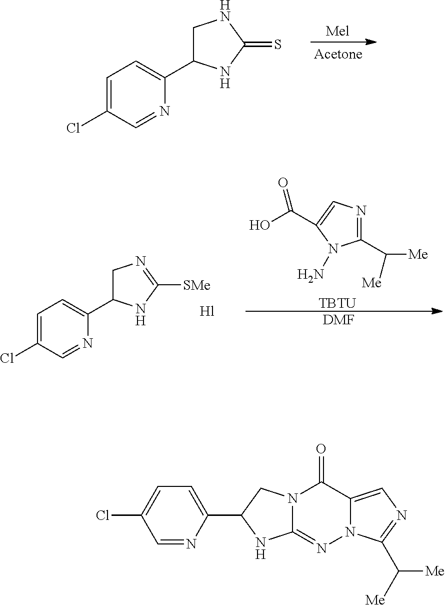

To solution of 5-chloro-2-(2-(methylthio)-4,5-dihydro-1H-imidazol-4-yl)pyridine hydroiodide (3.5 g, 13.57 mmol) in DMF (30 mL) was added 1-amino-2-isopropyl-1H-imidazole-5-carboxylic acid (3.9 g, 23.06 mmol) and TBTU (9.87 g, 30.74 mmol), Diisopropylethylamine (7.95 g, 61.48 mmol). The mixture was stirred at room temperature overnight. The crude product was purified by silica gel chromatography (eluted with DCM/methanol=20/1) to give the title compound as a white solid. (770 mg, 17%). LC-MS (m/z)=331 [M+H]⁺.

### 5-Chloro-2-(oxiran-2-yl)pyridine

To solution of 5-chloro-2-vinylpyridine (6 g, 42.99 mmol) in tert-Butanol (40 mL) and water (120 mL) was added NBS (9.18 g, 51.59 mmol). The mixture was stirred at room temperature for 1 h, then sodium hydroxide was added (10 N, 12.9 mL). The mixture was stirred at room temperature for 1 h. The crude product was extracted with Ethyl ether (100 mL×3). The combined organic layers were washed with brine (150 mL×3), dried over sodium sulfate, concentrated and purified by silica gel chromatography (eluted with dichloromethane/methanol=20/1) to give the title compound as a yellow oil (5.3 g, 79%). LC-MS (m/z)=156 [M+H]⁺.

### 2-Amino-1-(5-chloropyridin-2-yl)ethanol

A mixture of 5-chloro-2-(oxiran-2-yl)pyridine (5.3 g, 34.07 mmol) in ammonia hydrate (50 mL) was stirred at room temperature overnight. The crude product was purified by silica gel chromatography (eluted with dichloromethane/methanol=20/1) to give the title compound as a white solid. (5.2 g, 88%). LC-MS (m/z)=173 [M+H]⁺.

### tert-Butyl (2-(5-chloropyridin-2-yl)-2-hydroxyethyl)carbamate

To solution of 2-amino-1-(5-chloropyridin-2-yl)ethanol (5.2 g, 30.13 mmol) in DCM (100 mL) was added Di-tert-butyl dicarbonate (9.86 g, 45.2 mmol) and triethylamine (6.1 g, 60.26 mmol). The mixture was stirred at room temperature for 3 h. The crude product was purified by silica gel chromatography (eluted with dichloromethane/methanol=20/1) to give the title compound as a white solid. (7.9 g, 96%). LC-MS (m/z)=217 [M−56+H]⁺.

### tert-Butyl (2-(5-chloropyridin-2-yl)-2-(1, 3-dioxoisoindolin-2-yl) ethyl) carbamate

To a solution of tert-butyl (2-(5-chloropyridin-2-yl)-2-hydroxyethyl)carbamate (7.9 g, 28.97 mmol) in THF (100 mL) was added isoindoline-1,3-dione (5.11 g, 34.76 mmol) and triphenylphosphine (15.2 g, 57.94 mmol). The mixture was stirred at 0° C. and added diethyl azodicarboxylate (10.09 g, 57.94 mmol) under N₂. The mixture was stirred at room temperature overnight. The crude product was purified by silica gel chromatography (eluted with petroleum ether/ethyl acetate=5/1) to give the title compound as a white solid. (9.4 g, 84%). LC-MS (m/z)=302 [M−100+H]⁺.

### tert-Butyl (2-amino-2-(5-chloropyridin-2-yl)ethyl)carbamate

To a solution of tert-butyl (2-(5-chloropyridin-2-yl)-2-(1,3-dioxoisoindolin-2-yl) ethyl)carbamate (9.4 g, 23.39 mmol) in ethanol (150 mL) was added hydrazine (3.75 g, 116.95 mmol). The mixture was stirred at 75 C for 2 h. The crude product was purified by silica gel chromatography (eluted with dichloromethane/methanol=20/1) to give the title compound as a white solid. (5 g, 79%). LC-MS (m/z)=216 [M−56+H]⁺.

### 1-(5-Chloropyridin-2-yl) ethane-1,2-diamine bis(2,2,2-trifluoroacetate)

To a solution of tert-butyl (2-amino-2-(5-chloropyridin-2-yl) ethyl) carbamate (5 g, 18.4 mmol) in DCM (20 mL) was added 2, 2, 2-trifluoroacetic acid (20 mL). The mixture was stirred at room temperature overnight. Then it was concentrated in vacuo to give the title compound as a white solid (7.34 g, 100%). LC-MS (m/z)=172 [M+H]⁺.

### 4-(5-Chloropyridin-2-yl)imidazolidine-2-thione

To a solution of 1-(5-chloropyridin-2-yl)ethane-1,2-diamine bis(2,2,2-trifluoroacetate) (7.2 g, 18.06 mmol) in water (8 mL) was added trolamine (5.39 g, 36.12 mmol) and sulfur (60 mg, 1.81 mmol), carbon disulfide (1.79 g, 23.48 mmol). The mixture was stirred at 100° C. for 2 h. Some precipitate formed and was filtered to give the title compound. (3.3 g, 86%). LC-MS (m/z)=214 [M+H]⁺.

### 5-Chloro-2-(2-(methylthio)-4,5-dihydro-1H-imidazol-4-yl)pyridine hydroiodide

To a solution of 4-(5-chloropyridin-2-yl)imidazolidine-2-thione (3.3 g, 15.44 mmol) in acetone (8 mL) was added iodomethane (2.41 mg, 16.98 mmol). The mixture was stirred at 80° C. for 2 h. The solvents were removed in vacuo. The crude product was purified by silica gel chromatography (eluted with DCM/methanol=10/1) to give the title compound as a yellow solid (3.5 g, 100%). LC-MS (m/z)=228 [M+H]⁺.

The compounds of Reference Examples 14 and 15 were synthesized in a similar manner to Reference Example 13.

TABLE 3

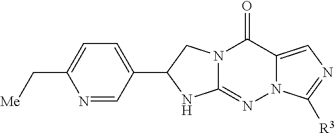

| No. | R³ | ¹H NMR |
|---|---|---|
| 14 | 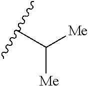 [SMILES: *C(C)C] | (400 MHz, CDCl₃): δ 8.55 (d, J = 4.0 Hz, 1H), 7.76 (s, 1H), 7.71- 7.69 (d, J = 8.0 Hz, 1H), 7.28 (t, J = 6.0 Hz, 1H), 5.63 (s, 1H), 5.15 (t, J = 8.0 Hz, 1H), 4.60 (t, J = 10.0 Hz, 1H), 3.95-3.90 (m, 1H), 3.40-3.37 (m, 1H), 2.90-2.84 (m, 2H), 1.39-1.28 (m, 9H). |
| 15 | 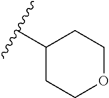 [SMILES: *C1CCOCC1] | (400 MHz, CDCl₃): δ 8.55-8.54 (m, 1H), 7.79 (s, 1H), 7.68-7.65 (m, 1H), 7.26-7.24 (m, 1H), 5.15-5.11 (m, 2H), 4.61-4.56 (m, 1H), 4.10-4.07 (m, 2H), 3.94-3.90 (m, 1H), 3.60-3.54 (m, 2H), 3.34- 3.28 (m, 1H), 2.89-2.83 (m, 2H), 2.11-2.03 (m, 2H), 1.92-1.88 (m, 2H), 1.32 (t, J = 8.0 Hz, 3H). |

### Reference Example 16

### 1-(Propan-2-yl)-6,7,8,9-tetrahydropyrazolo[3,4-d]pyrimido[1,2-a]pyrimidin-4(1H)-one

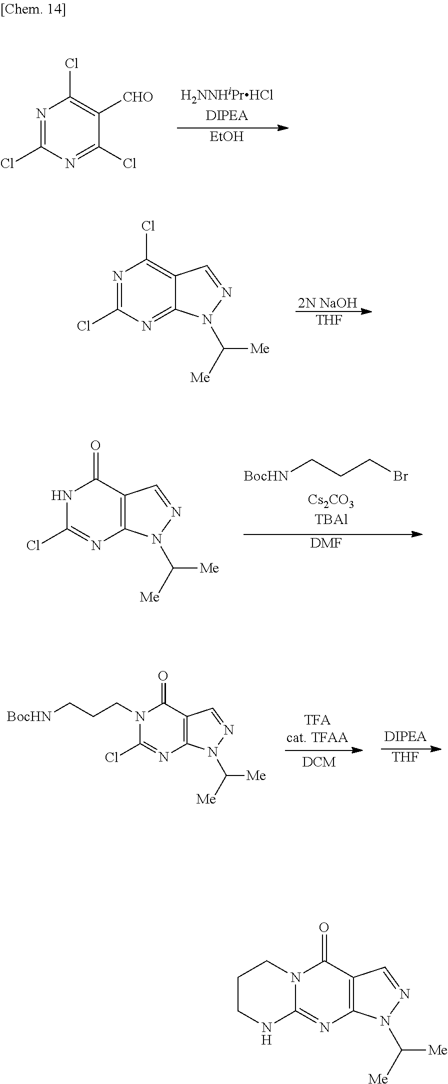

A mixture of compound tert-butyl {3-[6-chloro-4-oxo-1-(propan-2-yl)-1,4-dihydro-5H-pyrazolo[3,4-d]pyrimidin-5-yl]propyl}carbamate (910 mg, 2.46 mmol), trifluoroacetic acid (1.9 mL, 24.6 mmol) and trifluoroacetic anhydride (17 μL, 0.123 mmol) in dichloromethane (4.9 mL) was stirred at rt overnight. The reaction was concentrated in vacuo. A mixture of the above crude and diisopropylethylamine in THF was stirred at rt for 12 h. The resulting mixture was diluted with water, and extracted with ethyl acetate. The combined organic extracts were dried over sodium sulfate, filtered, and concentrated in vacuo. The residue was purified by silica gel chromatography (chloroform/methanol) to give the title compound (482 mg, 84%). LC-MS (m/z)=234 [M+H]⁺. ¹H NMR (400 MHz, CDCl₃): δ 7.89 (s, 1H), 5.26 (br s, 1H), 4.76-4.69 (m, 1H), 4.05-4.02 (m, 2H), 3.48-3.44 (m, 2H), 2.09-2.03 (m, 2H), 1.45 (d, J=6.7 Hz, 6H).

### 4,6-Dichloro-1-(propan-2-yl)-1H-pyrazolo[3,4-d]pyrimidine

To a solution of 2,4,6-trichloropyrimidine-5-carbaldehyde (6.00 g, 28.38 mmol) in ethanol (120 mL) was cooled to −78° C. and added isopropylhydrazine hydrochloride (3.14 g, 28.38 mmol) under a N₂ atmosphere. To a solution was added N-ethyldiisopropylamine (14.83 mL, 85.14 mmol) dropwise. The mixture was stirred at −78° C. for 2 h and then warmed up to room temperature and stirred at this temperature for 1 h. Water (60 mL) was added to the reaction solution and concentrated under reduced pressure. The mixture was extracted with ethyl acetate, washed with brine and dried over sodium sulfate. After concentration under reduced pressure, the residue was purified by silica gel column chromatography to give the title compound (6.15 g, 94%) as a white solid. LC-MS (m/z)=231 [M+H]⁺. ¹H NMR (400 MHz, CDCl₃): δ 8.14 (s, 1H), 5.18 (sept, J=6.8 Hz, 1H), 1.58 (d, J=6.8 Hz, 6H).

### 6-Chloro-1-(propan-2-yl)-1,5-dihydro-4H-pyrazolo[3,4-d]pyrimidin-4-one

To a solution of 4,6-dichloro-1-(propan-2-yl)-1H-pyrazolo[3,4-d]pyrimidine (6.14 g, 26.57 mmol) in THF (80 mL) was added 2 N sodium hydroxide (240 mL, 159.42 mmol) and the mixture was stirred at 50° C. for 12.5 h. After concentration under reduce pressure, the residue was added 5 N HCl (26 mL) and filtrated to give the title compound (5.53 g, 98%) as a white solid. LC-MS (m/z)=213 [M+H]⁺. ¹H NMR (400 MHz, CDCl₃): δ 10.82 (br, 1H), 8.10 (s, 1H), 5.01 (sept, J=6.6 Hz, 1H), 1.54 (d, J=6.6 Hz, 6H).

### tert-Butyl {3-[6-chloro-4-oxo-1-(propan-2-yl)-1,4-dihydro-5H-pyrazolo[3,4-d]pyrimidin-5-yl]propyl}carbamate

A mixture of compound 6-chloro-1-(propan-2-yl)-1,5-dihydro-4H-pyrazolo[3,4-d]pyrimidin-4-one (2.1 g, 9.88 mmol), cesium carbonate (4.82 g, 1.8 mmol), tetra-n-butylammonium iodide (365 mg, 0.988 mmol), and tert-butyl (3-bromopropyl)carbamate (2.35 g, 9.88 mmol) in DMF (33 mL) was stirred at rt for 2 days. The reaction was quenched with water, and extracted with ethyl acetate. The combined organic extracts were washed with brine, dried over sodium sulfate, filtered, and concentrated in vacuo. The residue was purified by silica gel chromatography (hexane/EtOAc) to give the title compound (910 mg, 25%). LC-MS (m/z)=370 [M+H]⁺.

### Reference Example 17

### 1-(propan-2-yl)-1,6,7,8-tetrahydro-4H-imidazo[1,2-a]pyrazolo[3,4-d]pyrimidin-4-one

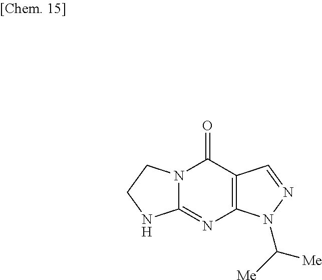 [SMILES: CC(C)n1ncc2c(=O)n3c(nc21)NCC3]

The titled compound was synthesized in a similar manner to Reference Example 16.

¹H NMR (400 MHz, CDCl₃): δ 7.92 (s, 1H), 5.21 (br, 1H), 4.77 (sept, J=6.7 Hz, 1H), 4.23 (t, J=8.4 Hz, 2H), 3.83 (t, J=8.4 Hz, 2H), 1.48 (d, J=6.7 Hz, 6H).

### Reference Example 18

### 8-(4-Chlorophenyl)-1-isopropyl-6,7,8,9-tetrahydropyrazolo[3,4-d]pyrimido[1,2-a]pyrimidin-4(1H)-one

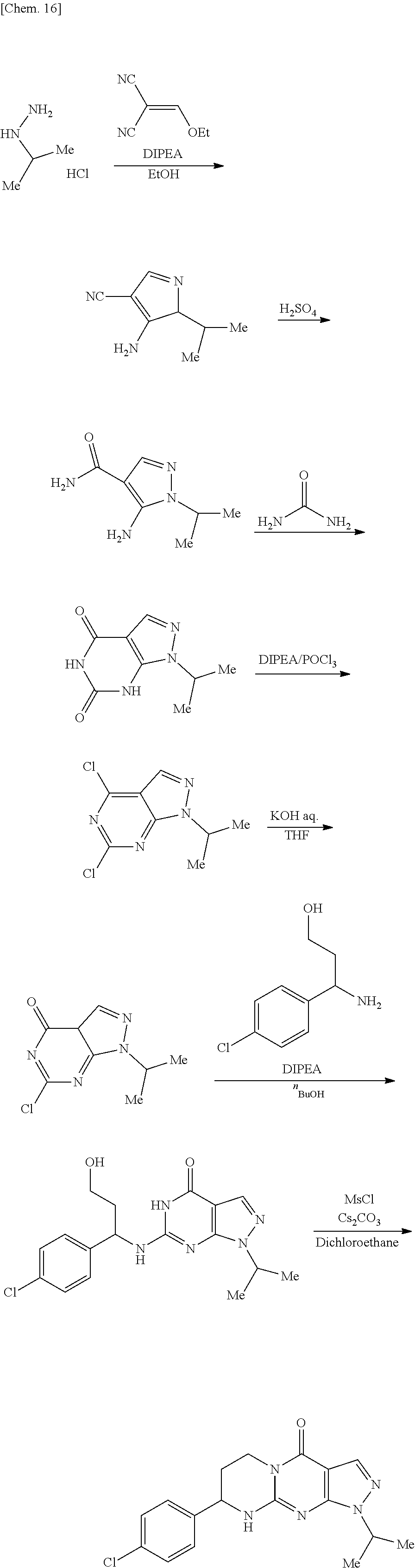

To a mixture of 6-{[1-(4-Chlorophenyl)-3-hydroxypropyl]amino}-1-(propan-2-yl)-1,5-dihydro-4H-pyrazolo[3,4-d]pyrimidin-4-one (205 mg, 0.57 mmol) in dichloroethane (5 mL) was added methanesulfonyl chloride (65 mg, 0.57 mmol), Cs₂CO₃ (557 mg, 1.71 mmol). The mixture was stirred at 60° C. for 12 h. Filtered and concentrated, the crude product was purified by Prep-TLC plate (eluted with DCM/MeOH=10/1) to give the title compound (100 mg, 51%) as a white solid. LC-MS (m/z)=344 [M+H]⁺. ¹H NMR (400 MHz, CDCl₃): δ 7.95 (s, 1H), 7.39 (d, J=8.0 Hz, 2H), 7.30 (d, J=8.0 Hz, 2H), 5.52 (s, 1H), 4.81-4.70 (m, 2H), 4.26-4.20 (m, 1H), 3.93-3.86 (m, 1H), 3.38-3.33 (m, 1H), 2.06-2.01 (m, 1H), 1.51-1.48 (m, 6H).

### 5-Amino-1-(propan-2-yl)-1H-pyrazole-4-carbonitrile

(Ethoxymethylidene)propanedinitrile (12.83 g, 105 mmol) and isopropylhydrazine hydrochloride (11.06 g, 100 mmol) were combined in EtOH (250 mL). Diisopropylethylamine (36.6 mL, 210 mmol) was added drop-wise, resulting in some warming of the reaction mixture. The reaction was allowed to stir for about 18 h at room temperature. Volatiles were then removed in vacuo, and the resulting viscous yellow oil was dissolved in dichloromethane and loaded onto a short column of silica gel. The column was eluted with dichloromethane (about 300 mL), followed by a 1:1 mixture of EtOAc and hexane (about 750 mL), and the EtOAc/hexanes eluant was concentrated under reduced pressure to provide the title compound (12.1 g, 81%) as a pale yellow solid. LC-MS (m/z)=151 [M+H]⁺. ¹H NMR (400 MHz, DMSO-d₆): δ 1.26 (d, J=6.6 Hz, 6H), 4.41 (sept, J=6.5 Hz, 1H), 6.52 (br, 2H), 7.53 (s, 1H).

### 5-Amino-1-(propan-2-yl)-1H-pyrazole-4-carboxamide

5-Amino-1-(propan-2-yl)-1H-pyrazole-4-carbonitrile (4.0 g, 27 mmol) was combined with concentrated sulfuric acid (about 10 mL) and stirred at room temperature for 2 h. The reaction was then poured onto ice, adjusted to pH 9 with concentrated aqueous ammonium hydroxide, and extracted with a mixture of dichloromethane and tetrahydrofuran. The organic layer was dried over magnesium sulfate, filtered and concentrated in vacuo to provide the title compound (3.02 g, 67%) as a pale gray solid. LC-MS (m/z)=169 [M+H]⁺.

¹H NMR (400 MHz, CD₃OD): δ 1.39 (d, J=6.6 Hz, 6H), 4.39 (sept, J=6.6 Hz, 1H), 7.69 (s, 1H).

### 1-(Propan-2-yl)-1H-pyrazolo[3,4-d]pyrimidine-4,6(5H,7H)-dione

A mixture of 5-amino-1-(propan-2-yl)-1H-pyrazole-4-carboxamide (4.2 g, 25 mmol) and urea (3.0 g, 50 mmol) was heated to 230° C. for 3 h. The reaction was cooled to room temperature, quenched with 20% NaOH/H₂O (W/W) (100 mL). The resulted mixture was stirred at room temperature for 10.0 h and neutralized with 1.5 M HCl aqueous solution. The resulted mixture was filtered to give the title compound (3.4 g, 70%) as white solid. LC-MS (m/z)=195 [M+H]⁺.

### 4,6-Dichloro-1-(propan-2-yl)-1H-pyrazolo[3,4-d]pyrimidine

To a mixture of 1-(propan-2-yl)-1H-pyrazolo[3,4-d]pyrimidine-4,6(5H,7H)-dione (3.4 g, 17.5 mmol) in phosphoryl trichloride (30 mL), was added N,N-diisopropylethylamine (3.4 g, 26.3 mmol). The reaction mixture was stirred at 120° C. for 3 h and cooled to room temperature. Excess of phosphoryl trichloride was concentrated. The residue obtained was poured into ice-water and neutralized with saturated NaHCO₃ aqueous solution. The resulted mixture was filtered to give the title compound (2.5 g, 62%). LC-MS (m/z)=231 [M+H]⁺.

### 6-Chloro-1-(propan-2-yl)-1,3a-dihydro-4H-pyrazolo[3,4-d]pyrimidin-4-one

To a mixture of 4,6-dichloro-1-(propan-2-yl)-1H-pyrazolo[3,4-d]pyrimidine (2.5 g, 10.8 mmol) in tetrahydrofuran (20 mL) was added 2.0 M KOH aqueous solution (20 mL, 40.0 mmol). The reaction mixture was stirred at 50° C. for 2.0 h and cooled to room temperature. The mixture was neutralized with 1.0 N HCl aqueous solution and white precipitation was formed. The resulted mixture was filtered to give the title compound (1.5 g, 66%) as white solid. LC-MS (m/z)=213 [M+H]⁺.

### 6-{[1-(4-Chlorophenyl)-3-hydroxypropyl]amino}-1-(propan-2-yl)-1,5-dihydro-4H-pyrazolo[3,4-d]pyrimidin-4-one

To a solution of 6-chloro-1-isopropyl-1H-pyrazolo[3,4-d]pyrimidin-4(5H)-one (197 mg, 0.93 mmol) in n-butyl alcohol (4 mL) was added Diisopropylethylamine (375 mg, 2.91 mmol) and 3-amino-3-(4-chlorophenyl)propan-1-ol (172 mg, 0.93 mmol) at room temperature. The mixture was stirred at 120° C. overnight. The crude product was purified by silica gel chromatography (eluted with DCM/MeOH=10/1) to give the title compound (214 mg, 64%) as a white solid. LC-MS (m/z)=362 [M+H]⁺.

### Reference Example 19

### 4-(Propan-2-yl)-7,8-dihydroimidazo[1,2-a]pyrimido[5,4-d]pyrimidin-10(6H)-one

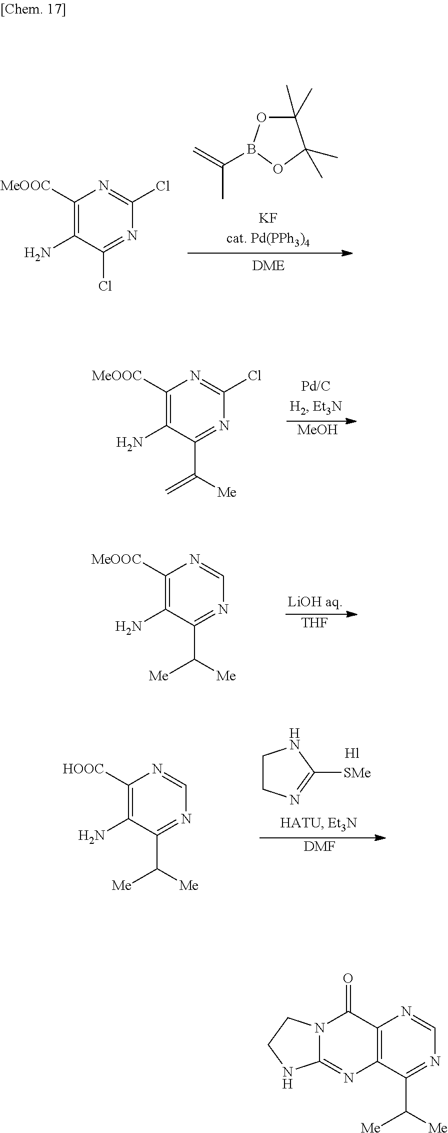

To a solution of 5-amino-6-(propan-2-yl)pyrimidine-4-carboxylic acid (130 mg, 0.90 mmol) and 2-(methylsulfanyl)-4,5-dihydro-1H-imidazole hydroiodide (125 mg, 1.076 mmol) in DMF (3 mL) were added HATU (409 mg, 1.076 mmol) and triethylamine (0.299 ml, 2.152 mmol). The mixture was stirred at room temperature overnight. The reaction mixture was concentrated and the crude product was washed with 2 M NaOH/AcOEt to give the title compound (0.135 g, 81%) as a white solid. LC-MS (m/z)=232 [M+H]⁺. ¹H NMR (400 MHz, CDCl₃): δ 8.12 (s, 1H), 7.54 (s, 1H), 3.56 (t, J=8.5 Hz, 2H), 3.23-3.16 (m, 1H), 3.08 (t, J=8.7 Hz, 2H), 0.57 (d, J=6.8 Hz, 6H).

### Methyl 5-amino-2-chloro-6-(prop-1-en-2-yl)pyrimidine-4-carboxylate

To a solution of methyl 5-amino-2,6-dichloropyrimidine-4-carboxylate (3.0 g, 13.51 mmol) and 4,4,5,5-tetramethyl-2-(prop-1-en-2-yl)-1,3,2-dioxaborolane (3.86 ml, 20.26 mmol) in DME (100 mL) and 10% KF aq. (25 ml) was added tetrakis(triphenylphosphine)palladium (1.56 g, 1.351 mmol). The reaction was heated overnight at 90° C. under nitrogen atmosphere. Upon completion, the reaction mixture was cooled and partitioned between ethyl acetate (50 mL) and brine (40 ml). The aqueous layer was extracted with EtOAc (80 mL×2), and the combined organic phases were dried with sodium sulfate and concentrated to dryness. The residue was purified by column chromatography on silica gel (elution with petroleum ether/ethyl acetate=100/0-50/50) to give the title compound (1.83 g, 59%) as a white solid. LCMS (m/z)=228 [M+H]⁺. ¹H NMR (400 MHz, CDCl₃): δ 6.09 (s, 2H), 5.66 (dd, J=1.6, 0.9 Hz, 1H), 5.50 (t, J=1.0 Hz, 1H), 3.97 (s, 3H), 2.15 (dd, J=1.7, 1.0 Hz, 3H).

### Methyl 5-amino-6-(propan-2-yl)pyrimidine-4-carboxylate

To methyl 5-amino-2-chloro-6-(prop-1-en-2-yl)pyrimidine-4-carboxylate (1.82 g, 7.959 mmol) in methanol (70 mL) and triethylamine (7 ml) under nitrogen was added palladium (10 wt. % on activated carbon) (1.0 g). The reaction mixture was deoxygenated under vacuum, and hydrogenated at 0.3 MPa overnight. Upon completion, the reaction mixture was filtered through a pad of Celite and washed with MeOH (40 mL×2). The filtrate was concentrated and the residue was purified by column chromatography on silica gel (elution with PE/EtOAc=100/0-50/50) to give the title compound (1.30 g, 84%) as a white solid. LC-MS (m/z)=196 [M+H]⁺.

### 5-Amino-6-(propan-2-yl)pyrimidine-4-carboxylic acid

To a solution of methyl 5-amino-6-(propan-2-yl)pyrimidine-4-carboxylate (0.5 g, 2.561 mmol) in THF (5.0 mL) and H₂O (5.0 ml) was added LiOH (0.322 g, 7.683 mmol). The mixture was stirred at ambient temperature for 2 h. The reaction mixture was acidified to pH 5 with 1M HCl, extracted with CHCl₃/MeOH (10/1, 30 mL×5) and dried (MgSO₄). The solvent was concentrated to give the title compound (0.447 g, 96%) as a white solid, which was used without further purification.

### Example 1

### 8-Isopropyl-1-((tetrahydro-2H-pyran-4-yl)methyl)-2,3-dihydrodiimidazo[2,1-c:1′,5′-f][1,2,4]triazin-5(1H)-one

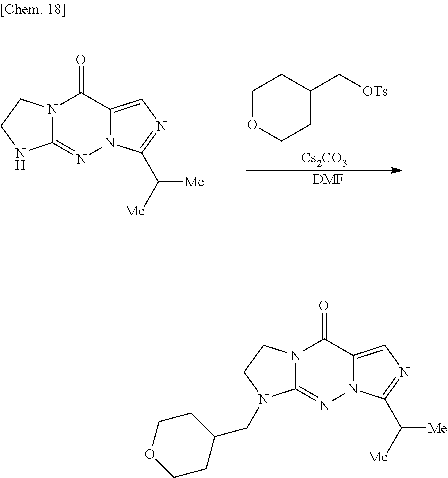

To a solution of (tetrahydro-2H-pyran-4-yl)methyl 4-methylbenzenesulfonate (101 mg, 0.38 mmol) and 8-isopropyl-2,3-dihydrodiimidazo[2,1-c:1′,5′-f][1,2,4]triazin-5(1H)-one (55 mg, 0.25 mmol) in N,N-dimethylformamide (1.5 mL) was added Cs₂CO₃ (163 mg, 0.5 mmol) the reaction mixture was stirred at 50° C. for 5 h and purified by prep-HPLC (MeCN and H₂O with 0.01% NH₃.H₂O as mobile phase) to give the title compound as a white solid (40 mg, 50%). LC-MS (m/z)=318 [M+H]⁺. ¹H NMR (400 MHz, CDCl₃): δ 1.38 (d, J=6.8 Hz, 6H), 1.42-1.49 (m, 2H) 1.66-1.69 (m, 2H), 1.98-2.04 (m, 1H), 3.27 (d, J=7.2 Hz, 2H), 3.37-3.45 (m, 3H), 3.70 (t, J=8.2 Hz, 2H), 4.01-4.04 (m, 2H), 4.11 (t, J=8.2 Hz, 2H), 7.73 (s, 1H).

### Example 2

### 1-Benzyl-9-isopropyl-1,2,3,4-tetrahydro-6H-imidazo[5,1-f]pyrimido[2,1-c][1,2,4]triazin-6-one

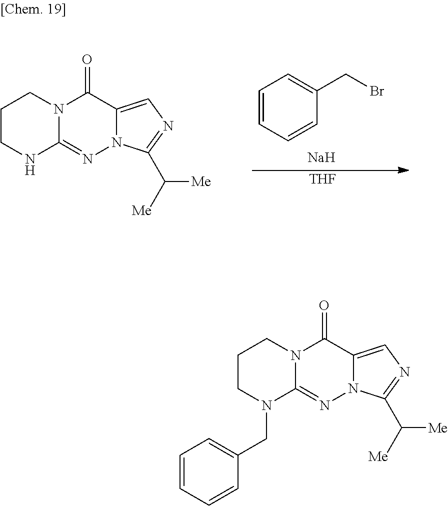

A solution of 9-(propan-2-yl)-1,2,3,4-tetrahydro-6H-imidazo[5,1-f]pyrimido[2,1-c][1,2,4]triazin-6-one (78 mg, 0.334 mmol) in anhydrous THF (2 mL) was added NaH (60% in mineral oil, 20 mg, 0.50 mmol) at 25° C. Then the mixture was stirred at 70° C. for 1 hour. After cooled to room temperature, (bromomethyl)benzene (85 mg, 0.50 mmol) was added. The mixture was heated to reflux for 8 hrs. The resulting mixture was quenched with water (20 mL) and extracted with EtOAc (15 mL×3). The combined organic layers were washed with brine dried over Na₂SO₄ and concentrated to dryness. The residue was purified by prep-HPLC (0.1% NH₃.H₂O as additive) to give the title compound (25 mg, 23%). LC-MS (m/z)=324 [M+H]⁺. ¹H NMR (400 MHz, CDCl₃): δ 7.73 (s, 1H), 7.37-7.25 (m, 5H), 4.76 (s, 2H), 4.03 (t, J=6.0 Hz, 2H), 3.42-3.35 (m, 3H), 2.09-2.03 (m, 2H), 1.33 (d, J=6.8 Hz, 6H).

### Example 3

### 11-Methyl-10-(oxan-4-ylmethyl)-6-(propan-2-yl)-1,5,7,8,10-pentaazatricyclo[7,4,0,0,3,7]trideca-3,5,8-trien-2-one

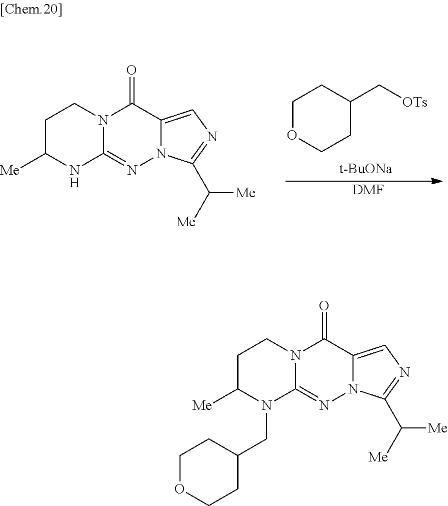

A solution of 9-isopropyl-2-methyl-3,4-dihydro-1H-imidazo[1,5-f]pyrimido[2,1-c][1,2,4]triazin-6(2H)-one (70 mg, 0.28 mmol), (tetrahydro-2H-pyran-4-yl)methyl 4-methylbenzene sulfonate (153 mg, 0.57 mmol) and t-BuONa (41 mg, 0.43 mmol) in anhydrous DMF (5 mL) was heated to 50° C. for 16 h. The reaction was cooled to 25° C., then quenched by aq. NH₄Cl. The title compound was purified through Pre-HPLC (11 mg, 11%). LCMS (m/z)=346 [M+H]⁺. ¹H NMR (400 MHz, CD₃OD): δ 1.20-1.21 (m, 3H), 1.24-1.27 (m, 6H), 1.53-1.62 (m, 2H), 1.86-1.91 (m, 1H), 2.02-2.10 (m, 1H), 2.14-2.24 (m, 1H), 2.94-2.99 (m, 1H), 3.24-3.34 (m, 4H), 3.57-3.70 (m, 4H), 3.84-3.88 (m, 2H), 4.18-4.23 (m, 1H), 7.47 (s, 1H).

The compounds of Examples 4 to 83 were synthesized in a similar manner to Example 1, 2, or 3.

TABLE 4

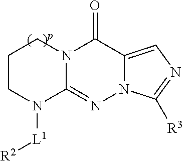

| No. | p | R²—L¹— | R³— | ¹H NMR |
|---|---|---|---|---|
| 4 | 0 | 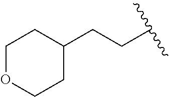 [SMILES: *CCC1CCOCC1] | 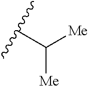 [SMILES: *C(C)C] | (400 MHz, CDCl₃): δ 1.33-1.39 (m, 8H), 1.59-1.63 (m, 3H), 1.69-1.73 (m, 2H), 3.36-3.43 (m, 5H), 3.63- 3.68 (m, 2H), 3.96-4.01 (m, 2H), 4.07-4.12 (m, 2H), 7.72 (s, 1H). |
| 5 | 0 | 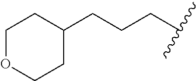 [SMILES: *CCCC1CCOCC1] | 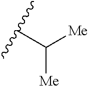 [SMILES: *C(C)C] | (400 MHz, CDCl₃): δ 1.18-1.31 (m, 10H), 1.43-1.48 (m, 1H), 1.53-1.64 (m, 4H), 3.25-3.35 (m, 5H), 3.59 (t, J = 8.0 Hz, 2H), 3.88-3.93 (m, 2H), 4.02 (t, J = 8.0 Hz, 2H), 7.65 (s, 1H). |
| 6 | 0 | 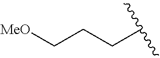 [SMILES: *CCCOC] | 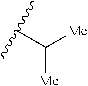 [SMILES: *C(C)C] | (400 MHz, CDCl₃): δ 1.38 (d, J = 6.8 Hz, 6H), 1.92-1.98 (m, 2H) 3.35 (s, 3H), 3.40-3.51 (m, 5H), 3.69 (t, J = 8.0 Hz, 2H), 4.09 (t, J = 8.0 Hz, 2H), 7.71 (s, 1H). |
| 7 | 0 | 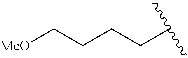 [SMILES: *CCCCOC] | 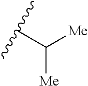 [SMILES: *C(C)C] | (400 MHz, CDCl₃): δ 1.36 (s, 3H), 1.38 (s, 3H), 1.65-1.70 (m, 2H), 1.74-1.78 (m, 2H), 3.35 (s, 3H), 3.37-3.46 (m, 5H), 3.66 (t, J = 8.0 Hz, 2H), 4.08 (t, J = 3.0 Hz, 2H), 7.72 (s, 1H). |
| 8 | 0 | 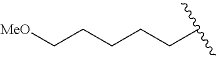 [SMILES: *CCCCCOC] | 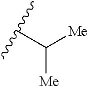 [SMILES: *C(C)C] | (400 MHz, CDCl₃): δ 1.36 (s, 3H), 1.37 (s, 3H), 1.44-1.49 (m, 2H), 1.64-1.74 (m, 4H), 3.34 (s, 3H), 3.34-3.42 (m, 5H), 3.64-3.68 (m, 2H), 4.06-4.10 (m, 2H), 7.72 (s, 1H). |
| 9 | 0 | 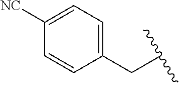 [SMILES: *Cc1ccc(C#N)cc1] | 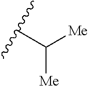 [SMILES: *C(C)C] | (400 MHz, CDCl₃): δ 1.36 (d, J = 6.8 Hz, 6 H), 3.37-3.44 (m, 1 H), 3.60 (t, J = 8.0 Hz, 2 H), 4.12 (t, J = 8.0 Hz, 2 H), 4.60 (s, 2 H), 7.49 (d, J = 8.0 Hz, 2 H), 7.69 (d. J = 8.0 Hz, 2H), 7.76 (s, 1 H). |
| 10 | 0 | 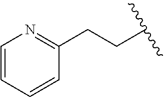 [SMILES: *CCc1ccccn1] | 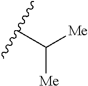 [SMILES: *C(C)C] | (400 MHz, CDCl₃): δ 1.29 (s, 3H), 1.31 (s, 3H), 3.10 (t, J = 7.0 Hz, 2H), 3.29-3.37 (m, 1H), 3.53 (t, J = 8.0 Hz, 2H), 3.73 (t, J = 7.0 Hz, 2H), 3.95 (t, J = 8.0 Hz, 2H), 7.08-7.12 (m, 1H), 7.15-7.18 (m, 1H), 7.54- 7.59 (m, 1H), 7.64 (s, 1 H), 8.47- 8.49 (m, 1H). |
| 11 | 0 | 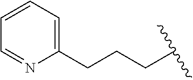 [SMILES: *CCCc1ccccn1] | 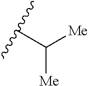 [SMILES: *C(C)C] | (400 MHz, CDCl₃: δ 1.37 (d, J = 6.7 Hz, 6H), 2.12-2.20 (m, 2H), 2.90 (t, J = 7.6 Hz, 2H), 3.35-3.42 (m, 1H), 3.44 (t, J = 7.1 Hz, 2H), 3.65 (t, J = 8.0 Hz, 2H), 4.00 (t, J = 8.0 Hz, 2H), 7.12-7.17 (m, 2H), 7.54-7.57 (m, 1H), 7.71 (s, 1H), 8.54 (d, J = 4.6 Hz, 1H). |
| 12 | 0 | 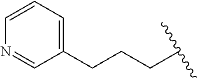 [SMILES: *CCCc1cccnc1] | 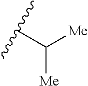 [SMILES: *C(C)C] | (400 MHz, CDCl₃): δ 1.37 (d, J = 7.2 Hz, 6H), 1.99-2.07 (m, 2H), 2.75 (t, J = 7.6 Hz, 2H), 3.43-3.45 (m, 3H), 3.65 (t, J = 8.0 Hz, 2H), 4.07 (t, J = 7.6 Hz, 2H), 7.21-7.24 (m, 1H), 7.54-7.56 (m, 1H), 7.73 (s, 1H), 8.48-8.50 (m, 2H). |
| 13 | 0 | 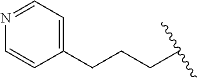 [SMILES: *CCCc1ccncc1] | 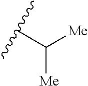 [SMILES: *C(C)C] | (400 MHz, CDCl₃): δ 1.37 (d. J = 7.2 Hz, 6H), 2.00-2.08 (m, 2H), 2.74 (t, J = 8.0 Hz, 2H), 3.35-3.42 (m, 3H), 3.65 (t, J = 8.4 Hz, 2H), 4.08 (t, J = 8.0 Hz, 2H), 7.16 (d, J = 6.0 Hz, 2H), 7.73 (s, 1H), 8.52 (d, J = 5.6 Hz, 2H). |
| 14 | 1 | Me— | 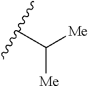 [SMILES: *C(C)C] | (400 MHz, CDCl₃): δ 7.72 (s, 1H), 4.07-3.94 (m, 2H), 3.52-3.33 (m, 3H), 3.10 (s, 3H), 2.15-2.20 (m, 2H), 1.38 (d, J = 6.4 Hz, 6H). |
| 15 | 1 | ⁿPr— | 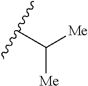 [SMILES: *C(C)C] | (400 MHz, CDCl₃): δ 7.70 (s, 1H), 4.02-3.99 (m, 2H), 3.49-3.39 (m, 5H), 2.08-2.05 (m, 2H), 1.77-1.71 (m, 2H), 1.38 (d, J = 7.2 Hz, 6H), 0.98 (t, J = 7.4 Hz, 3H). |
| 16 | 1 | 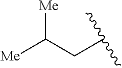 [SMILES: *CC(C)C] | 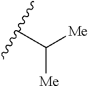 [SMILES: *C(C)C] | (400 MHz, CDCl₃): δ 7.70 (s, 1H), 4.02-3.99 (m, 2H), 3.45-3.32 (m, 5H), 2.25-2.21 (m, 1H), 2.10-2.04 (m, 2H), 1.37 (d, J = 7.2 Hz, 6H), 0.97 (d, J = 6.4 Hz, 6H). |
| 17 | 1 | ⁿBu— | 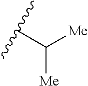 [SMILES: *C(C)C] | (400 MHz, CD₃OD): δ 0.99-1.02 (m, 3H), 1.35-1.37 (m, 6H), 1.40-1.46 (m, 2H), 1.70-1.78 (m, 2H), 2.05- 2.11 (m, 2H), 3.41-3.49 (m, 3H), 3.54-3.57 (m, 2H), 3.97-4.00 (m, 2H), 7.57 (s, 1H). |
| 18 | 1 | ⁿPen— | 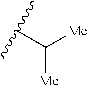 [SMILES: *C(C)C] | (400 MHz, CDCl₃): δ 7.71 (s, 1H), 4.01 (t, J = 6.0 Hz, 2H), 3.49 (t, J = 7.6 Hz, 2H), 3.43-3.37 (m, 3H), 2.08-2.05 (m, 2H), 1.73-1.69 (m, 2H), 1.39-1.34 (m, 10H), 0.94 (t, J = 7.0 Hz, 3H). |
| 19 | 1 | 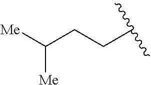 [SMILES: *CCC(C)C] | 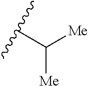 [SMILES: *C(C)C] | (400 MHz, CDCl₃): δ 7.71 (s, 1H), 4.03-4.00 (m, 2H), 3.56-3.52 (m, 2H), 3.44-3.41 (m, 3H), 2.12-2.06 (m, 2H), 1.64-1.62 (m, 3H), 1.41- 1.39 (m, 6H), 1.01-0.99 (m, 6H). |
| 20 | 1 | 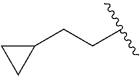 [SMILES: *CCC1CC1] | 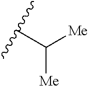 [SMILES: *C(C)C] | (400 MHz, CD₃OD): δ −0.01-0.00 (m, 2H), 0.35 (s, 2H), 0.60-0.62 (m, 1H), 1.21-1.22 (m, 6H), 1.48-1.52 (m, 2H), 1.91-1.98 (m, 2H), 3.27- 3.31 (m, 1H), 3.35-3.39 (m, 2H), 3.47-3.51 (m, 2H), 3.83-3.86 (m, 2H), 7.43 (s, 1H). |
| 21 | 1 | 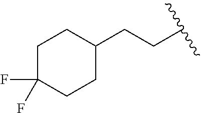 [SMILES: *CCC1CCC(F)(F)CC1] | 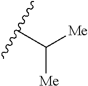 [SMILES: *C(C)C] | (400 MHz, CDCl₃): δ 1.37-1.41 (m, 9H), 1.66-1.87 (m, 6H) 2.02-2.12 (m, 4H), 3.33-3.43 (m, 3H), 3.52- 3.56 (m, 2H), 4.01 (t, J = 6.0 Hz, 2H), 7.72 (s, 1H). |
| 22 | 1 | 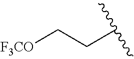 [SMILES: *CCOC(F)(F)F] | 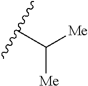 [SMILES: *C(C)C] | (400 MHz, CDCl₃): δ 7.73 (s, 1H), 4.40-4.30 (m, 2H), 4.08-3.95 (m, 2H), 3.88-3.78 (m, 2H), 3.60-3.50 (m, 2H), 3.40-3.26 (m, 1H), 2.15- 2.03 (m, 2H), 1.36 (d, J = 7.2 Hz, 6H). |
| 23 | 1 | 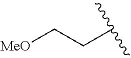 [SMILES: *CCOC] | 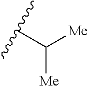 [SMILES: *C(C)C] | (400 MHz, CDCl₃): δ 7.70 (s, 1H), 4.01-3.98 (m, 2H), 3.71 (s, 4H), 3.56-3.53 (m, 2H), 3.38-3.35 (m, 4H), 2.09-2.03 (m, 2H), 1.36 (d, J = 7.2 Hz, 6H). |
| 24 | 1 | 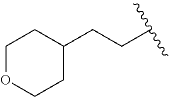 [SMILES: *CCC1CCOCC1] | 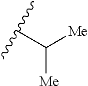 [SMILES: *C(C)C] | (400 MHz, CDCl₃): δ 7.72 (s, 1H), 4.02-3.97 (m, 4H), 3.57-3.53 (m, 2H), 3.43-3.37 (m, 5H), 2.09-2.06 (m, 2H), 1.69-1.55 (m, 5H), 1.43- 1.37 (m, 8H). |
| 25 | 1 | 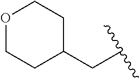 [SMILES: *CC1CCOCC1] | 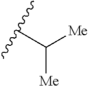 [SMILES: *C(C)C] | (400 MHz, CDCl₃): δ 7.69 (s, 1H), 3.98-3.95 (m, 4H), 3.43-3.31 (m, 7H), 2.40-2.30 (m, 1H), 2.16-2.04 (m, 2H), 1.63-1.59 (m, 2H), 1.40- 1.34 (m, 8H). |
| 26 | 1 | 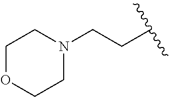 [SMILES: *CCN1CCOCC1] | 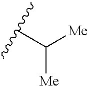 [SMILES: *C(C)C] | (400 MHz, DMSO-d₆): δ 1.26-1.28 (m, 6H), 1.94-2.00 (m, 2H), 2.45 (s, 4H), 2.59-2.62 (m, 2H), 3.26-3.31 (m, 1H), 3.43-3.44 (m, 2H), 3.55- 3.58 (m, 6H), 3.85-3.87 (m, 2H), 7.51 (s, 1H). |
| 27 | 1 | 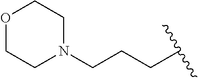 [SMILES: *CCCN1CCOCC1] | 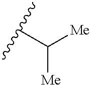 [SMILES: *C(C)C] | (400 MHz, CDCl₃): δ 1.36-1.38 (m, 6H), 1.92-1.93 (m, 2H), 2.02-2.11 (m, 2H), 2.42-2.47 (m, 6H), 3.33- 3.40 (m, 1H), 3.42-3.45 (m, 2H), 3.56-3.58 (m, 2H), 3.72-3.74 (m, 4H), 3.99-4.02 (m, 2H), 7.70 (s, 1H). |
| 28 | 1 | 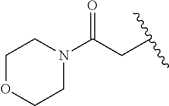 [SMILES: *CC(=O)N1CCOCC1] | 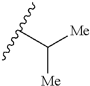 [SMILES: *C(C)C] | (400 MHz, CDCl₃): δ 1.35 (s, 3H), 1.37 (s, 3H), 2.14-2.20 (m, 2H), 3.26-3.33 (m, 1H), 3.53-3.58 (m, 4H), 3.64 (t, J = 4.6 Hz, 2H), 3.73 (t, J = 4.6 Hz, 2H), 3.77 (t, J = 4.8 Hz, 2H), 4.07 (t, J = 6.0 Hz, 2H), 4.26 (s, 2H), 7.70 (s, 1H). |
| 29 | 1 | 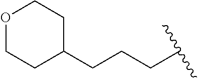 [SMILES: *CCCC1CCOCC1] | 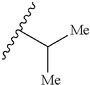 [SMILES: *C(C)C] | (400 MHz, CDCl₃): δ 1.25-1.29 (m, 4H), 1.35-1.39 (m, 6H), 1.45-1.58 (m, 1H), 1.62-1.78 (m, 4H), 2.05- 2.11 (m, 2H), 3.34-3.51 (m, 7H), 3.95-4.03 (m, 4H), 7.72 (s, 1H). |
| 30 | 1 | 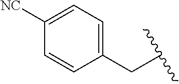 [SMILES: *Cc1ccc(C#N)cc1] | 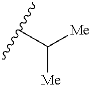 [SMILES: *C(C)C] | (400 MHz, CDCl₃): δ 7.73 (s, 1H), 7.66 (d, J = 8.0 Hz, 2H), 7.47 (d, J = 8.0 Hz, 2H), 4.80 (s, 2H), 4.07 (t, J = 6.0 Hz, 2H), 3.46 (t, J = 6.0 Hz, 2H), 3.27 (sept, J = 6.8 Hz, 1H), 2.16- 2.09 (m, 2H), 1.27 (d, J = 6.8 Hz, 6H). |
| 31 | 1 | 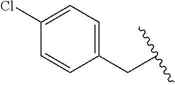 [SMILES: *Cc1ccc(Cl)cc1] | 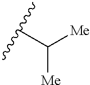 [SMILES: *C(C)C] | (400 MHz, CDCl₃): δ 7.73 (s, 1H), 7.35-7.25 (m, 4H), 4.71 (s, 2H), 4.07-4.00 (m, 2H), 3.41-3.29 (m, 3H), 2.13-2.02 (m, 2H), 1.32 (d, J = 7.2 Hz, 6H). |
| 32 | 1 | 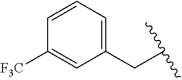 [SMILES: *Cc1cccc(C(F)(F)F)c1] | 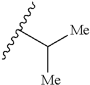 [SMILES: *C(C)C] | (400 MHz, CDCl₃): δ 7.75 (s, 1H), 7.70-7.46 (m, 4H), 4.79 (s, 2H), 4.07 (t, J = 6.0 Hz, 2H), 3.47 (t, J = 6.4 Hz, 2H), 3.34 (sept, J = 6.8 Hz, 1H), 2.15-2.09 (m, 2H), 1.31 (d, J = 6.8 Hz, 6H). |
| 33 | 1 | 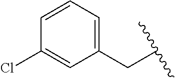 [SMILES: *Cc1cccc(Cl)c1] | 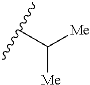 [SMILES: *C(C)C] | (400 MHz, CDCl₃): δ 7.72 (s, 1H), 7.37 (s, 1H), 7.31-7.20 (m, 3H), 4.70 (s, 2H), 4.04 (t, J = 6.0 Hz, 2H), 3.44-3.30 (m, 3H), 2.13-2.03 (m, 2H), 1.32 (d, J = 6.8 Hz, 6H). |
| 34 | 1 | 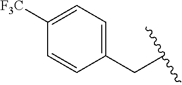 [SMILES: *Cc1ccc(C(F)(F)F)cc1] | 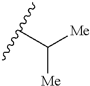 [SMILES: *C(C)C] | (400 MHz, CDCl₃): δ 7.73 (s, 1H), 7.62 (d, J = 8.0 Hz, 2H), 7.48 (d, J = 8.0 Hz, 2H), 4.80 (s, 2H), 4.06 (t, J = 6.0 Hz, 2H), 3.44 (t, J = 6.0 Hz, 2H), 3.32 (sept, J = 6.8 Hz, 1H), 2.13- 2.08 (m, 2H), 1.29 (d, J = 6.8 Hz, 6H). |
| 35 | 1 | 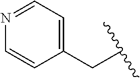 [SMILES: *Cc1ccncc1] | 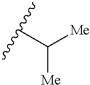 [SMILES: *C(C)C] | (400 MHz, CDCl₃): δ 8.64-8.55 (m, 2H), 7.73 (s, 1H), 7.26-7.24 (m, 2H), 4.74 (s, 2H), 4.08 (t, J = 6.0 Hz, 2H), 3.47 (t, J = 6.0 Hz, 2H), 3.25 (sept, J = 7.2 Hz, 1H), 2.17-2.11 (m, 2H), 1.25 (d, J = 7.2 Hz, 6H). |
| 36 | 1 | 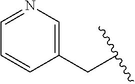 [SMILES: *Cc1cccnc1] | 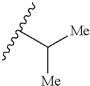 [SMILES: *C(C)C] | (400 MHz, DMSO-d₆): δ 8.63 (s, 1H), 8.47-8.46 (m, 1H), 7.83-7.81 (m, 1H), 7.51 (s, 1H), 7.38-7.36 (m, 1H), 4.72 (s, 2H), 3.91 (t, J = 6.0 Hz, 2H), 3.51 (t, J = 6.0 Hz. 2H), 3.22 (sept, J = 7.2 Hz, 1H), 2.08-2.02 (m, 2H), 1.16 (d, J = 7.2 Hz, 6H). |
| 37 | 1 | 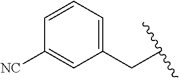 [SMILES: *Cc1cccc(C#N)c1] | 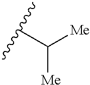 [SMILES: *C(C)C] | (400 MHz, CDCl₃): δ 7.72 (s, 1H), 7.67 (s, 1H), 7.62-7.59 (m, 2H), 7.50-7.46 (m, 1H), 4.76 (s, 2H), 4.06 (t, J = 6.0 Hz, 2H), 3.47 (t, J = 6.0 Hz, 2H), 3.30 (sept, J = 7.2 Hz, 1H), 2.15-2.09 (m, 2H), 1.29 (d, J = 7.2 Hz, 6H). |
| 38 | 1 | 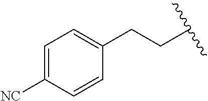 [SMILES: *CCc1ccc(C#N)cc1] | 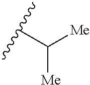 [SMILES: *C(C)C] | (400 MHz, CDCl₃): δ 1.43 (d, J = 6.8 Hz, 6H), 1.99-2.02 (m, 2H), 3.12 (t, J = 7.6 Hz, 2H), 3.29 (t, J = 6.0 Hz, 2H), 3.42-3.45 (m, 1H), 3.73 (t, J = 7.2 Hz, 2H), 3.99 (t, J = 6.0 Hz, 2H), 7.37 (d, J = 8.4 Hz, 2H), 7.64 (d, J = 8.8 Hz, 2H), 7.75 (s, 1H). |
| 39 | 1 | 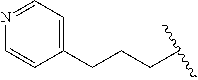 [SMILES: *CCCc1ccncc1] | 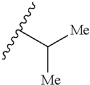 [SMILES: *C(C)C] | (400 MHz, CD₃OD): δ 1.30 (d, J = 6.8 Hz, 6H), 2.04-2.17 (m, 4H), 2.80 (t, J = 7.6 Hz, 2H), 3.24-3.29 (m, 1H), 3.48 (t, J = 5.6 Hz, 2H), 3.60 (t, J = 7.6 Hz, 2H), 3.96 (t, J = 6.0 Hz, 2H), 7.34 (d, J = 5.6 Hz, 2H), 7.57 (s, 1H), 8.42 (d, J = 6.0 Hz, 2H). |
| 40 | 1 | 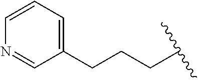 [SMILES: *CCCc1cccnc1] | 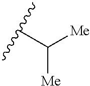 [SMILES: *C(C)C] | (400 MHz, CD₃OD): δ 1.30 (d, J = 6.8 Hz, 6H), 2.04-2.15 (m, 4H), 2.79 (t, J = 8.0 Hz, 2H), 3.23-3.33 (m, 1H), 3.48 (t, J = 6.0 Hz, 2H), 3.61 (t, J = 7.2 Hz, 2H), 3.96 (t, J = 6.4 Hz, 2H), 7.36-7.39 (m, 1H), 7.57 (s, 1H), 7.75-7.77 (m, 1H), 8.38-8.40 (m, 1H), 8.44 (d, J = 2.0 Hz, 1H). |
| 41 | 1 | 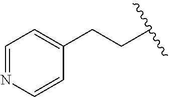 [SMILES: *CCc1ccncc1] | 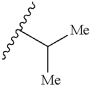 [SMILES: *C(C)C] | (400 MHz, CDCl₃): δ 1.42 (s, 3H), 1.44 (s, 3H), 1.98-2.04 (m, 2H), 3.06 (t, J = 7.3 Hz, 2H), 3.31 (t, J = 6.0 Hz, 2H), 3.41-3.48 (m, 1H). 3.74 (t, J = 7.3 Hz, 2H), 3.99 (t, J = 5.8 Hz, 2H), 7.19-7.20 (m, 2H), 7.75 (s, 1H), 8.56-8.58 (m, 2H). |
| 42 | 1 | 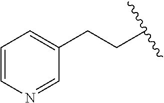 [SMILES: *CCc1cccnc1] | 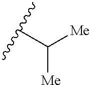 [SMILES: *C(C)C] | (400 MHz, DMSO-d₆): δ 1.44 (d, J = 7.2 Hz, 6H), 2.01-2.04 (m, 2H), 3.07 (t, J = 7.4 Hz, 2H), 3.33 (t, J = 6.0 Hz, 2H), 3.43-3.48 (m, 1H), 3.73 (t, J = 7.4 Hz, 2H), 4.00 (t, J = 6.0 Hz, 2H), 7.29 (d, J = 5.2 Hz, 1H), 7.58 (d, J = 7.6 Hz, 1H), 7.75 (s, 1H), 8.52-8.55 (s, 2 H). |
| 43 | 1 | 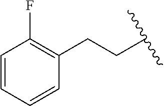 [SMILES: *CCc1ccccc1F] | 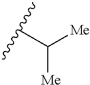 [SMILES: *C(C)C] | (400 MHz, CDCl₃): δ 1.41 (s, 3H), 1.43 (s, 3H), 1.96-2.06 (m, 2H), 3.10 (t, J = 7.3 Hz, 2H), 3.31 (t, J = 6.0 Hz, 2H), 3.42-3.49 (m, 1H), 3.74 (t, J = 7.4 Hz, 2H), 3.97 (t, J = 6.0 Hz, 2H), 7.03-7.11 (m, 2H), 7.21-7.26 (m, 2H), 7.33 (s, 1H). |
| 44 | 1 | 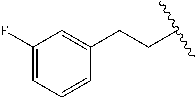 [SMILES: *CCc1cccc(F)c1] | 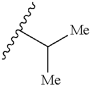 [SMILES: *C(C)C] | (400 MHz, CDCl₃): δ 1.43 (d, J = 7.2 HZ, 6H), 1.97-2.03 (m, 2H), 3.05 (t, J = 7.2 Hz, 2H), 3.28 (t, J = 6.4 Hz, 2H), 3.42-3.49 (m, 1H), 3.72 (t, J = 7.4 Hz, 2H), 3.98 (t, J = 6.0 Hz, 2H), 3.94-7.03 (m, 3H), 7.28-7.32 (m, 1H), 7.74 (s, 1H). |
| 45 | 1 | 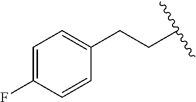 [SMILES: *CCc1ccc(F)cc1] | 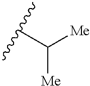 [SMILES: *C(C)C] | (400 MHz, CDCl₃): δ 1.44 (d, J = 6.8 Hz, 6H), 1.98-2.02 (m, 2H), 3.01-3.05 (t, J = 7.4 Hz, 2H), 3.27 (t, J = 6.0 Hz, 2H), 3.44-3.48 (m, 1H), 3.68-3.72 (t, J = 7.4 Hz, 2H), 3.98 (t, J = 6.0 Hz, 2H), 7.01-7.05 (m, 2H), 7.20-7.27 (m, 2H), 7.75 (s, 1H). |
| 46 | 1 | 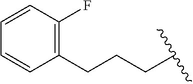 [SMILES: *CCCc1ccccc1F] | 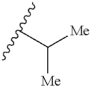 [SMILES: *C(C)C] | (400 MHz, DMSO-d₆): δ 1.36 (d, J = 7.2 Hz, 6H), 2.03-2.10 (m, 4H), 2.75 (t, J = 7.4 Hz, 2H), 3.25-3.32 (m, 1H), 3.41 (t, J = 6.0 Hz, 2H), 3.56 (t, J = 7.6 Hz, 2H), 3.98 (t, J = 6.0 Hz, 2H), 7.01-7.09 (m, 2H), 7.18-7.21 (m, 2H), 7.71 (s, 1H). |
| 47 | 1 | 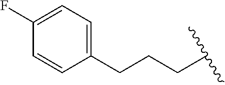 [SMILES: *CCCc1ccc(F)cc1] | 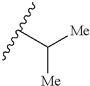 [SMILES: *C(C)C] | (400 MHz, DMSO-d₆): δ 1.37 (d, J = 7.2 Hz, 6H), 2.00-2.07 (m, 4H), 2.69 (t, J = 7.6 Hz, 2H), 3.25-3.32 (m, 1H), 3.40 (t, J = 6 Hz, 2H), 3.53 (t, J = 7.8 Hz, 2H), 3.99 (t, J = 5.8 Hz, 2H), 6.99 (t, J = 8.6 Hz, 2H), 7.14- 7.18 (m, 2H), 7.72 (s, 1H). |
| 48 | 1 | 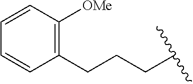 [SMILES: *CCCc1ccccc1OC] | 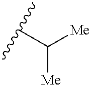 [SMILES: *C(C)C] | (400 MHz, CDCl₃): δ 7.70 (s, 1H), 7.19-7.12 (m, 2H), 6.88-6.83 (m, 2H), 3.97-3.94 (m, 2H), 3.79 (s, 3H), 3.57-3.53 (m, 2H), 3.40-3.37 (m, 2H), 3.29 (m, 1H), 2.71-2.67 (m, 2H), 2.05-1.99 (m, 4H), 1.35 (d, J = 7.2 Hz, 6H). |
| 49 | 1 | 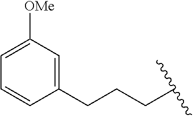 [SMILES: *CCCc1cccc(OC)c1] | 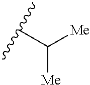 [SMILES: *C(C)C] | (400 MHz, CD₃OD): δ 1.30-1.32 (m, 6H), 2.00-2.08 (m, 4H), 2.68-2.72 (m, 2H), 3.26-3.28 (m, 1H), 3.41- 3.43 (m, 2H), 3.56-3.60 (m, 2H), 3.73(s, 3H), 3.88-3.89 (m, 2H), 6.72- 6.75 (m, 2H), 6.78-6.80 (m, 1H), 7.14-7.18 (m, 1H), 7.56 (s, 1H). |
| 50 | 1 | 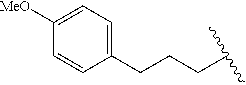 [SMILES: *CCCc1ccc(OC)cc1] | 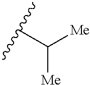 [SMILES: *C(C)C] | (400 MHz, CDCl₃): δ 7.73 (s, 1H), 7.13-7.11 (m, 2H), 6.85-6.83 (m, 2H), 3.98-3.95 (m, 2H), 3.81 (s, 3H), 3.56-3.52 (m, 2H), 3.40-3.37 (m, 2H), 3.34-3.31 (m, 1H), 2.69-2.65 (m, 2H), 2.06-2.02 (m, 4H), 1.39- 1.37 (m, 6H). |
| 51 | 2 | 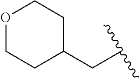 [SMILES: *CC1CCOCC1] | 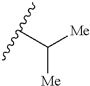 [SMILES: *C(C)C] | (400 MHz, DMSO-d₆): δ 1.34-1.38 (m, 2H), 1.41 (d, J = 6.8 Hz, 6H), 1.70-1.74 (m, 2H), 1.84-1.85 (m, 4H), 1.85-1.96 (m, 1H), 3.20-3.24 (m, 4H), 3.37-3.46 (m, 3H), 4.02 (dd, J = 11.0, 3.4 Hz, 2H), 4.04-4.17 (m, 2H), 7.78 (s, 1H). |
| 52 | 2 | 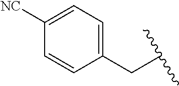 [SMILES: *Cc1ccc(C#N)cc1] | 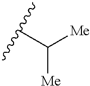 [SMILES: *C(C)C] | (400 MHz, CDCl₃): δ 1.33 (d, J = 6.8 Hz, 6H), 1.78-1.86 (m, 1H), 3.16-3.18 (m, 2H), 3.36-3.43 (m, 1H), 4.24-4.26 (s, 2H), 4.54 (s, 2H), 7.52-7.54 (m, 2H), 7.67-7.69 (m, 2H), 7.77 (s, 1H). |
| 53 | 0 | ⁿBu— | 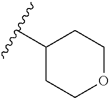 [SMILES: *C1CCOCC1] | (400 MHz, CDCl₃): δ 1.00 (t, J = 7.2 Hz, 3H), 1.42 (sext, J = 7.4 Hz, 2H), 1.65 (quint, J = 7.4 Hz, 2H), 1.90- 1.94 (m, 2H), 2.02-2.12 (m, 2H), 3.29-3.38 (m, 3H), 3.55-3.69 (m, 4H), 4.09 (t, J = 7.8 Hz, 4H), 7.72 (s, 1H). |
| 54 | 0 | 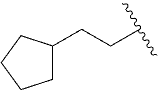 [SMILES: *CCC1CCCC1] | 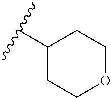 [SMILES: *C1CCOCC1] | (400 MHz, CDCl₃): δ 1.14-1.22 (m, 2H), 1.51-1.71 (m, 7H), 1.80-1.94 (m, 4H), 2.02-2.12 (m, 2H), 3.29- 3.39 (m, 3H), 3.55-3.61 (m, 2H), 3.67 (t, J = 8.0 Hz, 2H), 4.09 (t, J = 7.8 Hz, 4H), 7.72 (s, 1H). |
| 55 | 0 | 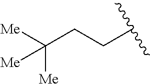 [SMILES: *CCC(C)(C)C] | 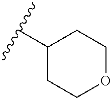 [SMILES: *C1CCOCC1] | (400 MHz, CDCl₃): δ 1.00 (s, 9H), 1.54-1.58 (m, 2H), 1.92-2.12 (m, 4H), 3.28-3.39 (m, 3H), 3.52-3.59 (m, 2H), 3.68 (t, J = 7.8 Hz, 2H), 4.08 (t, J = 7.8 Hz, 4H), 7.72 (s, 1H). |
| 56 | 0 | 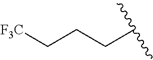 [SMILES: *CCCC(F)(F)F] | 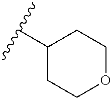 [SMILES: *C1CCOCC1] | (400 MHz, CDCl₃): δ 1.88-2.12 (m, 6H), 2.18-2.30 (m, 2H), 3.28-3.36 (m, 1H), 3.45 (t, J = 7.2 Hz, 2H), 3.55-3.61 (m, 2H), 3.69 (t, J = 8.0 Hz, 2H), 4.07-4.14 (m, 4H), 7.74 (s, 1H). |
| 57 | 0 | 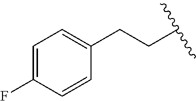 [SMILES: *CCc1ccc(F)cc1] | 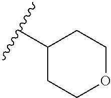 [SMILES: *C1CCOCC1] | (400 MHz, CDCl₃): δ 1.88-1.94 (m, 2H), 2.04-2.15 (m, 2H), 2.97 (t, J = 7.2 Hz, 2H), 3.30-3.37 (m, 1H), 3.55-3.63 (m, 6H), 4.04-4.12 (m, 4H), 7.02 (t, J = 8.4 Hz, 2H), 7.21 (dd, J = 8.4, 6.4 Hz, 2H), 7.27 (s, 1 H). |
| 58 | 0 | 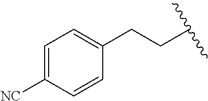 [SMILES: *CCc1ccc(C#N)cc1] | 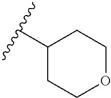 [SMILES: *C1CCOCC1] | (400 MHz, CDCl₃): δ 1.88-1.92 (m, 2H), 2.05-2.16 (m, 2H), 3.08 (t, J = 7.2 Hz, 2H), 3.29-3.36 (m, 1H), 3.56-3.66 (m, 6H), 4.04-4.12 (m, 4H), 7.39 (d, J = 8.0 Hz, 2H), 7.64 (d, J = 8.0 Hz, 2H), 7.72 (s, 1H). |
| 59 | 1 | 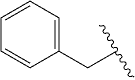 [SMILES: *Cc1ccccc1] | 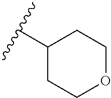 [SMILES: *C1CCOCC1] | (400 MHz, DMSO-d₆): δ 1.78-1.82 (m, 2H), 1.95-2.14 (m, 4H), 3.21- 3.29 (m, 1H), 3.46-3.54 (m, 4H), 4.00-4.08 (m, 4H), 4.74 (s, 2H), 7.29-7.39 (m, 5H), 7.74 (s, 1H). |
| 60 | 1 | 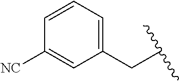 [SMILES: *Cc1cccc(C#N)c1] | 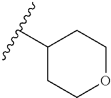 [SMILES: *C1CCOCC1] | (400 MHz, CDCl₃): δ 1.76-1.77 (m, 2H), 1.94-2.04 (m, 2H), 2.12-2.18 (m, 2H), 3.19 (tt, J = 3.8, 11.5 Hz, 1H), 3.45-3.46 (m, 2H), 3.51 (t, J = 6.0 Hz, 2H), 4.00-4.04 (m, 2H), 4.08 (t, J = 5.8 Hz, 2H), 4.75 (s, 2H), 7.47-7.51 (m, 1H), 7.59-7.62 (m, 2H), 7.66 (s, 1H), 7.74 (s, 1H). |
| 61 | 1 | 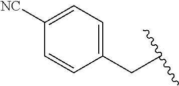 [SMILES: *Cc1ccc(C#N)cc1] | 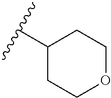 [SMILES: *C1CCOCC1] | (400 MHz, CDCl₃): δ 7.75 (s, 1H), 7.68 (d, J = 8.4 Hz, 2H), 7.46 (d, J = 8.4 Hz, 2H), 4.79 (m, 2H), 4.11-4.08 (m, 2H), 4.04-3.97 (m, 2H), 3.53- 3.43 (m, 4H), 3.16 (m, 1H), 2.18- 2.15 (m, 2H), 2.00-1.96 (m, 2H), 1.74-1.70 (m, 2H). |
| 62 | 1 | 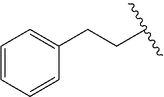 [SMILES: *CCc1ccccc1] | 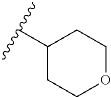 [SMILES: *C1CCOCC1] | (400 MHz, DMSO-d₆): δ 1.98-2.05 (m, 4H), 2.10-2.20 (m, 2H), 3.02- 3.06 (m, 2H), 3.31-3.34 (m, 2H), 3.36-3.44 (m, 1H), 3.59-3.65 (m, 2H), 3.70-3.74 (m, 2H), 3.98-4.01 (m, 2H), 4.12-4.16 (m, 2H), 7.27- 7.29 (m, 3H), 7.34-7.37 (m, 2H), 7.75 (s, 1H). |
| 63 | 1 | 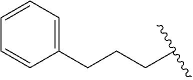 [SMILES: *CCCc1ccccc1] | 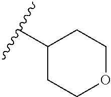 [SMILES: *C1CCOCC1] | (400 MHz, CDCl₃): δ 7.74 (s, 1H), 7.32-7.28 (m, 2H), 7.23-7.19 (m, 3H), 4.12-4.08 (m, 2H), 4.00-3.95 (m, 2H), 3.61-3.55 (m, 4H), 3.41- 3.38 (m, 2H), 3.33-3.27 (m, 1H), 2.76-2.70 (m, 2H), 2.16-2.05 (m, 6H), 1.96-1.93 (m, 2H). |

### Examples 64 and 65

TABLE 5

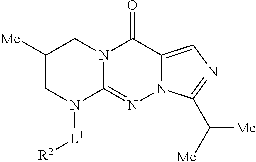

| No. | R²—L¹— | ¹H NMR |
|---|---|---|
| 64 | 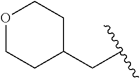 [SMILES: *CC1CCOCC1] | (400 MHz, CDCl₃): δ 1.14 (d, J = 6.8 Hz, 3H), 1.39 (d, J = 6.8 Hz, 6H), 1.43-1.44 (m, 2H) 1.63-1.66 (m, 2H), 2.16-2.23 (m, 2H), 3.11-3.21 (m, 2H), 3.33-3.49 (m, 6H), 4.00-4.04 (m, 2H), 4.45-4.49 (m, 1H), 7.72 (s, 1H). |
| 65 | 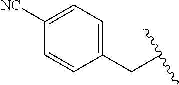 [SMILES: *Cc1ccc(C#N)cc1] | (400 MHz, CDCl₃): δ 1.12 (d, J = 6.8 Hz, 3H), 1.28 (d, J = 5.6 Hz, 6H), 2.20-2.26 (m, 1H), 3.10-3.15 (m, 1H), 3.20-3.31 (m, 2H), 3.37-3.41 (m, 1H), 4.50-4.54 (m, 1H), 4.71-4.75 (m, 1H), 4.82-4.86 (m, 1H), 7.46 (d, J = 8.0 Hz, 2H), 7.66 (d, J = 8.0 Hz, 2H), 7.73 (s, 1H). |

### Examples 66 to 72

TABLE 6

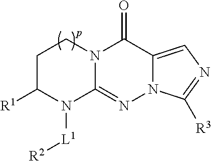

| No. | p | R¹— | R²—L¹— | R³— | ¹H NMR |
|---|---|---|---|---|---|
| 66 | 0 | 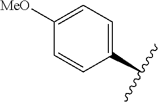 | Et— | 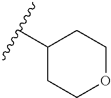 [SMILES: *C1CCOCC1] | (400 MHz, CDCl₃): δ 7.76 (s, 1H), 7.27-7.26 (m, 2H), 6.95 (d, J = 8.3 Hz, 2H), 4.85 (t, J= 8.4 Hz, 1H), 4.45 (dd, J = 11.3, 9.1 Hz, 1H), 4.10 (d, J = 11.5 Hz, 2H), 3.84-3.79 (m, |
| | | | | | 4H), 3.60 (t, J = 11.6 Hz, 2H), |
| | | | | | 3.54-3.45 (m, 1H), 3.41-3.33 |
| | | | | | (m, 1H), 3.03-2.94 (m, 1H), |
| | | | | | 2.17-2.05 (m, 2H), 1.95 (d, J = |
| | | | | | 12.2 Hz, 2H), 1.13 (t, J = |
| | | | | | 7.1 Hz, 3H). |
| 67 | 0 | 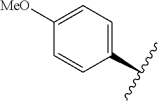 | ⁿPr— | 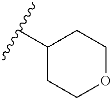 [SMILES: *C1CCOCC1] | (400 MHz, CDCl₃): δ 7.76 (s, 1H), 7.27-7.23 (m, 2H), 6.96- 6.93 (m, 2H), 4.86-4.82 (m, 1H), 4.47-4.42 (m, 1H), 4.12- 4.08 (m, 2H), 3.91-3.84 (m, 4H), 3.62-3.57 (m, 2H), 3.41- |
| | | | | | 3.32 (m, 2H), 2.96-2.86 (m, |
| | | | | | 1H), 2.17-2.04 (m, 2H), 1.97- |
| | | | | | 1.93 (m, 2H), 1.61-1.51 (m, |
| | | | | | 2H), 0.89 (t, J = 7.2 Hz, 3H). |
| 68 | 0 | 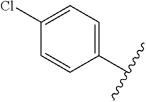 [SMILES: *c1ccc(Cl)cc1] | Me— | 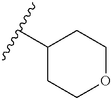 [SMILES: *C1CCOCC1] | (400 MHz, CDCl₃): δ 7.78 (s, 1H), 7.45-7.43 (m, 2H), 7.31- 7.27 (m, 2H), 4.73 (t, J = 8.0 Hz, 1H), 4.54-4.49 (m, 1H), 4.12-4.09 (m, 2H), 3.82- 3.77 (m, 1H), 3.63-3.57 (m, |
| | | | | | 2H), 3.57-3.36 (m, 1H), 2.81 |
| | | | | | (s, 3H), 2.14-2.07 (m, 2H), |
| | | | | | 1.97-1.93 (m, 2H). |
| 69 | 1 | 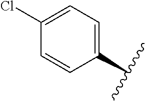 | Me— | 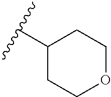 [SMILES: *C1CCOCC1] | (400 MHz, CDCl₃): δ 7.79 (s, 1H), 7.41 (d, J = 8.4 Hz, 2H), 7.17 (d, J = 8.4 Hz, 2H), 4.64- 4.66 (m, 1H), 4.30-4.36 (m, 1H), 4.10-4.15 (m, 2H), 3.53- 3.64 (m, 3H), 3.37-3.44 (m, |
| | | | | | 1H), 3.03 (s, 3H), 2.36-2.45 |
| | | | | | (m, 1H), 2.06-2.22 (m, 3H), |
| | | | | | 1.97-1.22 (m, 2H). |
| 70 | 1 | 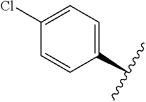 | Et— | 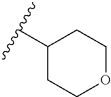 [SMILES: *C1CCOCC1] | (400 MHz, CDCl₃): δ 7.79 (s, 1H), 7.42-7.39 (m, 2H), 7.29- 7.18 (m, 2H), 4.76-4.74 (m, 1H), 4.44-4.16 (m, 1H), 4.15- 4.09 (m, 2H), 3.93-3.88 (m, 1H), 3.64-3.58 (m, 2H), 3.42- |
| | | | | | 3.33 (m, 2H), 3.10-3.05 (m, |
| | | | | | 1H), 2.32 (s, 1H), 2.21-1.99 |
| | | | | | (m, 3H), 1.99-1.97 (m, 2H), |
| | | | | | 1.62-1.26 (m, 3H). |
| 71 | 1 | 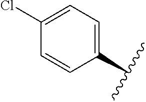 | ⁿPr— | 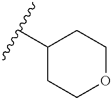 [SMILES: *C1CCOCC1] | (400 MHz, CDCl₃): δ 7.78 (s, 1H), 7.41-7.38 (m, 2H), 7.29- 7.15 (m, 2H), 4.78-4.75 (m, 1H), 4.43-4.14 (m, 1H), 4.15- 4.09 (m, 2H), 3.93-3.87 (m, 1H), 3.64-3.56 (m, 2H), 3.42- |
| | | | | | 3.33 (m, 2H), 2.96-2.89 (m, |
| | | | | | 1H), 2.38-2.31 (m, 1H), 2.21- |
| | | | | | 1.99 (m, 3H), 1.99-1.97 (m, |
| | | | | | 2H), 1.88-1.79 (m, 2H), 1.62- |
| | | | | | 1.26 (m, 3H). |
| 72 | 1 | 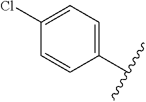 [SMILES: *c1ccc(Cl)cc1] | Me— | 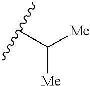 [SMILES: *C(C)C] | (400 MHz, CDCl₃): δ 7.76 (s, 1H), 7.39 (d, J = 8.4 Hz, 2H), 7.16 (d, J = 8.0 Hz, 2H), 4.62 (t, J = 5.2 Hz, 1H), 4.32-4.28 (m, 1H), 3.57-3.45 (m, 2H), 3.01 (s, 3H), 2.39-2.36 (m, |
| | | | | | 1H), 2.09-2.04 (m, 1H), 1.41 |
| | | | | | (t, J = 6.8 Hz, 6H). |

### Examples 73 to 81

TABLE 7

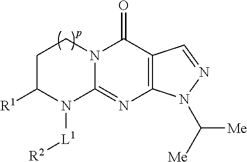

| No. | p | R¹ | R²—L¹— | ¹H NMR |
|---|---|---|---|---|
| 73 | 0 | H | 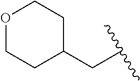 [SMILES: *CC1CCOCC1] | (400 MHz, CDCl₃): δ 7.89 (s, 1H), 4.83 (sept, J = 6.6 Hz, 1H), 4.13 (t, J = 8.5 Hz, 2H), 4.05-3.97 (m, 2H), 3.73 (t, J = 8.5 Hz, 2H), 3.45-3.36 (m, 2H), 3.35 (d, J = 7.3 Hz, 2H), |
| | | | | 2.05-1.90 (m, 1H), 1.70-1.61 (m, |
| | | | | 2H), 1.49 (d, J = 6.6 Hz, 6H), 1.47- |
| | | | | 1.40 (m, 2H). |
| 74 | 0 | H | 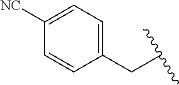 [SMILES: *Cc1ccc(C#N)cc1] | (400 MHz, CDCl₃): δ 7.92 (s, 1H), 7.68 (d, J = 8.1 Hz, 2H), 7.47 (d, J = 8.1 Hz, 2H), 4.85 (sept, J = 6.6 Hz, 1H), 4.70 (s, 2H), 4.14 (t, J = 8.5 Hz, 2H), 3.62 (t, J = 8.5 Hz, 2H), 1.50 (d, J = 6.6 Hz, 6H). |
| 75 | 0 | H | 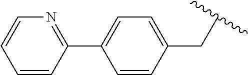 [SMILES: *Cc1ccc(-c2ccccn2)cc1] | (400 MHz, CDCl₃): δ 8.68 (dq, J = 4.9, 0.9 Hz, 1H), 7.99-7.97 (m, 2H), 7.90 (s, 1H), 7.73 (ddt, J = 18.1, 9.3, 2.5 Hz, 2H), 7.43 (d, J = 8.3 Hz, 2H), 7.26-7.21 (m, 1H), |
| | | | | 4.92-4.85 (m, 1H), 4.68 (s, 2H), |
| | | | | 4.10-4.06 (m, 2H), 3.61-3.57 (m, |
| | | | | 2H), 1.50 (d, J = 6.6 Hz, 6H). |
| 76 | 0 | H | 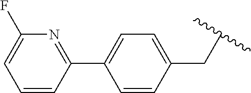 [SMILES: *Cc1ccc(-c2cccc(F)n2)cc1] | (400 MHz, CDCl₃): δ 8.52 (d, J = 2.9 Hz, 1H), 7.93 (d, J = 8.0 Hz, 3H), 7.90 (s, 1H), 7.70 (dd, J = 8.8, 4.1 Hz, 1H), 7.49-7.41 (m, 3H), 4.92-4.85 (m, 1H), 4.67 (s, 2H), 4.08 (t, J = 8.4 Hz, 2H), 3.59 (t, J = |
| | | | | 8.5 Hz, 2H), 1.50 (d, J = 6.8 Hz, |
| | | | | 6H). |
| 77 | 1 | H | ^{iso}Bu— | (400 MHz, CDCl₃): δ 7.85 (s, 1H), |
| | | | | 4.79-4.72 (m, 1H), 4.04-4.01 (m, |
| | | | | 2H), 3.47-3.43 (m, 4H), 2.22-2.15 |
| | | | | (m, 1H), 2.07-2.01 (m, 2H), 1.48 |
| | | | | (d, J = 7.3 Hz, 6H), 0.94 (d, J = |
| | | | | 6.7 Hz, 6H). |
| 78 | 1 | H | 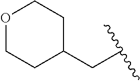 [SMILES: *CC1CCOCC1] | (400 MHz, CDCl₃): δ 7.86 (s, 1H), 4.78-4.71 (m, 1H), 4.04-3.97 (m, 4H), 3.55 (d, J = 7.3 Hz, 2H), 3.46 (t, J = 5.8 Hz, 2H), 3.39-3.33 (m, 2H), 2.16-2.02 (m, 3H), 1.62-1.53 (m, 2H), 1.51-1.36 (m, 8H). |
| 79 | 1 | H | 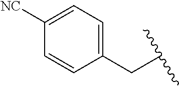 [SMILES: *Cc1ccc(C#N)cc1] | (400 MHz, CDCl₃): δ 7.88 (s, 1H), 7.64-7.62 (m, 2H), 7.43-7.41 (m, 2H), 4.94 (s, 2H), 4.73-4.67 (m, 1H), 4.10-4.07 (m, 2H), 3.44-3.41 (m, 2H), 2.10-2.04 (m, 2H), 1.41 (d, J = 6.7 Hz, 6H). |
| 80 | 1 | H | 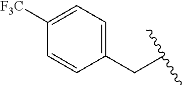 [SMILES: *Cc1ccc(C(F)(F)F)cc1] | (400 MHz, CDCl₃): δ 7.89 (s, 1H), 7.60-7.58 (m, 2H), 7.45-7.43 (m, 2H), 4.95 (s, 2H), 4.77-4.70 (m, 1H), 4.09-4.07 (m, 2H), 3.42 (t, J = 6.1 Hz, 2H), 2.08-2.02 (m, 2H), 1.42 (d, J = 6.7 Hz, 6H). |
| 81 | 1 | 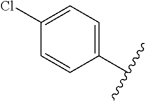 [SMILES: *c1ccc(Cl)cc1] | Me | (400 MHz, CDCl₃): δ 7.93 (s, 1H), 7.39-7.36 (m, 2H), 7.14 (d, J = 8.4 Hz, 2H), 4.86-4.93 (m, 1H), 4.69-4.67 (m, 1H), 4.48-4.42 (m, 1H), 3.47-3.40 (m, 1H), 3.14 (s, 3H), 2.37-2.29 (m, 1H), 2.11-2.05 (m, 1H), 1.53 (t, J = 7.2 Hz, 6H). |

### Examples 82 and 83

TABLE 8

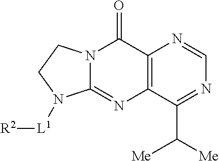

| No. | R²—L¹— | ¹H NMR |
|---|---|---|
| 82 | 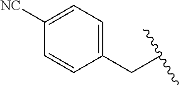 [SMILES: *Cc1ccc(C#N)cc1] | (400 MHz, CDCl₃): δ 9.00 (s, 1H), 7.63 (d, J = 8.0 Hz, 2H), 7.48 (d, J = 7.8 Hz, 2H), 4.75 (s, 2H), 4.25 (dd, J = 9.3, 7.8 Hz, 2H), 4.01-3.94 (m, 1H), 3.66 (t, J = 8.5 Hz, 2H, ), 1.32 (d, J = 6.8 Hz, 6H). |
| 83 | 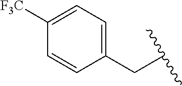 [SMILES: *Cc1ccc(C(F)(F)F)cc1] | (400 MHz, CDCl₃): δ 9.01 (s, 1H), 7.67 (d, J = 8.5 Hz, 2H), 7.48 (d, J = 8.5 Hz, 2H), 4.74 (s, 2H), 4.26 (t, J = 8.4 Hz, 2H), 3.98-3.91 (m, 1H), 3.67 (t, J = 8.4 Hz, 2H), 1.31 (d, J = 6.8 Hz, 6H). |

### Examples 84 and 85

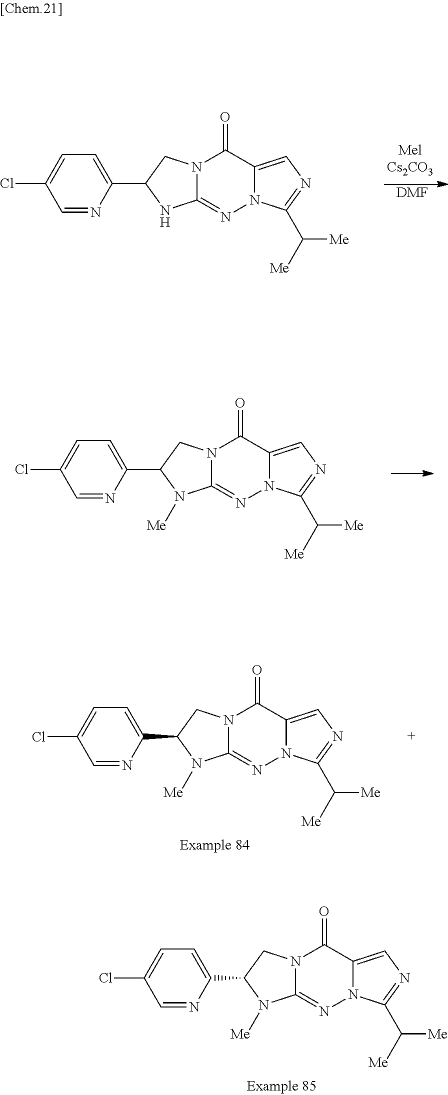

### (R)-2-(5-Chloropyridin-2-yl)-8-isopropyl-1-methyl-2,3-dihydrodiimidazo[2,1-c:1′,5′-f][1,2,4]triazin-5(1H)-one

The chiral separation of 2-(5-chloropyridin-2-yl)-8-isopropyl-1-methyl-2,3-dihydrodiimidazo[2,1-c:1′,5′-f][1,2,4]triazin-5(1H)-one (600 mg, 1.7 mmol) gave the title compound (155 mg, 25%). Retention Time: 6.46 min./Method A. LC-MS (m/z)=345 [M+H]⁺. ¹H NMR (400 MHz, CDCl₃): δ 8.62 (d, J=4.0 Hz, 1H), 7.79-7.76 (m, 2H), 7.32 (d, J=8.0 Hz, 1H), 4.93-4.89 (m, 1H), 4.51-4.46 (m, 1H), 4.06-4.02 (m, 1H), 3.49-3.42 (m, 1H), 2.85 (s, 3H), 1.41-1.38 (m, 6H).

### (S)-2-(5-Chloropyridin-2-yl)-8-isopropyl-1-methyl-2,3-dihydrodiimidazo[2,1-c:1′,5′-f][1,2,4]triazin-5(1H)-one

The chiral separation of 2-(5-chloropyridin-2-yl)-8-isopropyl-1-methyl-2,3-dihydrodiimidazo[2,1-c:1′,5′-f][1,2,4]triazin-5(1H)-one (600 mg, 1.7 mmol) gave the title compound (140 mg, 23%). Retention Time: 3.96 min./Method A. LC-MS (m/z)=345 [M+H]⁺. ¹H NMR (400 MHz, CDCl₃): δ 8.62 (d, J=4.0 Hz, 1H), 7.78-7.75 (m, 2H), 7.32 (d, J=8.0 Hz, 1H), 4.92-4.88 (m, 1H), 4.50-4.45 (m, 1H), 4.06-4.01 (m, 1H), 3.49-3.42 (m, 1H), 2.85 (s, 3H), 1.40-1.38 (m, 6H).

### 2-(5-Chloropyridin-2-yl)-8-isopropyl-1-methyl-2,3-dihydrodiimidazo[2,1-c:1′,5′-f][1,2,4]triazin-5(1H)-one

To solution of 2-(5-chloropyridin-2-yl)-8-isopropyl-2,3-dihydrodiimidazo[2,1-c:1′,5′-f][1,2,4]triazin-5(1H)-one (770 mg, 2.33 mmol) in DMF (10 mL) added cesium carbonate (1.52 g, 4.66 mmol) and iodomethane (360 mg, 2.56 mmol), the mixture was stirred at room temperature for 2 h. The crude product was purified by silica gel chromatography (eluted with DCM/methanol=50/1) to give the title compound as a white solid (600 mg, 75%). LC-MS (m/z)=345 [M+H]⁺.

The compounds of Examples 86 to 99 were synthesized in a similar manner to Examples 84 and 85.

TABLE 9

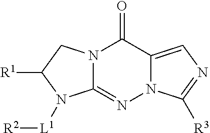

| | | | | | SFC: |
|---|---|---|---|---|---|
| | | | | | retention |
| | | | | | time/ |
| No. | R¹ | R²—L¹— | R³ | ¹H NMR | Method |
| 86 | 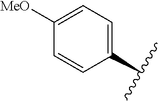 | Me— | 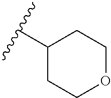 [SMILES: *C1CCOCC1] | (400 MHz, CDCl₃): δ 7.78 (s, 1H), 7.29-7.27 (m, 2H), 6.99-6.96 (m, 2H), 4.71 (t, J = 8.6 Hz, 1H), 4.52-4.47 (m, 1H), 4.13-4.10 (m, 2H), 3.86 (s, 3H), 3.83- | 4.05 min./ Method B |
| | | | | 3.80 (m, 1H), 3.64-3.58 | |
| | | | | (m, 2H), 3.40 (m, 1H), | |
| | | | | 2.79 (s, 3H), 2.14-2.11 (m, | |
| | | | | 2H), 1.98-1.95-1.98 (m, | |
| | | | | 2H) | |
| 87 | 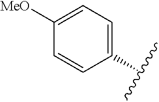 | Me— | 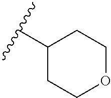 [SMILES: *C1CCOCC1] | (400 MHz, CDCl₃): δ 7.78 (s, 1H), 7.29-7.27 (m, 2H), 6.99-6.96 (m, 2H), 4.73- 4.68 (m, 1H), 4.52-4.47 (m, 1H), 4.13-4.10 (m, 2H), 3.86 (s, 3H), 3.80- | 2.26 min./ Method B |
| | | | | 3.83 (m, 1H), 3.64-3.58 | |
| | | | | (m, 2H), 3.40 (m, 1H), | |
| | | | | 2.79 (s, 3H), 2.14-2.09 (m, | |
| | | | | 2H), 1.98-1.94 (m, 2H). | |
| 88 | 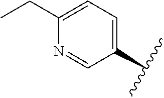 | Et— | 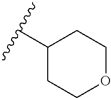 [SMILES: *C1CCOCC1] | (400 MHz, CDCl₃): δ 8.53-8.52 (m, 1H), 7.77 (s, 1H), 7.63-7.61 (m, 1H), 7.28-7.27 (m, 1H), 4.95- 4.91 (m, 1H), 4.53-4.48 (m, 1H), 4.12-4.09 (m, | 4.41 min./ Method C |
| | | | | 2H), 3.86-3.81 (m, 1H), | |
| | | | | 3.63-3.51 (m, 3H), 3.40- | |
| | | | | 3.36 (m, 1H), 3.01-2.96 | |
| | | | | (m, 1H), 2.91-2.85 (m, | |
| | | | | 2H), 2.13-2.09 (m, 2H), | |
| | | | | 1.96-1.93 (m, 2H), 1.36- | |
| | | | | 1.32 (m, 3H), 1.18-1.14 | |
| | | | | (m, 3H). | |
| 89 | 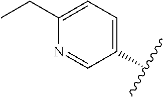 | Et— | 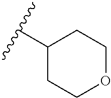 [SMILES: *C1CCOCC1] | (400 MHz, CDCl₃): δ 8.53-8.52 (m, 1H), 7.77 (s, 1H), 7.63-7.61 (m, 1H), 7.28-7.27 (m, 1H), 4.95- 4.91 (m, 1H), 4.53-4.48 (m, 1H), 4.12-4.08 (m, | 3.04 min./ Method C |
| | | | | 2H), 3.86-3.81 (m, 1H), | |
| | | | | 3.63-3.51 (m, 3H), 3.37- | |
| | | | | 3.36 (m, 1H), 3.01-2.96 | |
| | | | | (m, 1H), 2.91-2.85 (m, | |
| | | | | 2H), 2.13-2.09 (m, 2H), | |
| | | | | 1.96-1.93 (m, 2H), 1.35- | |
| | | | | 1.32 (m, 3H), 1.18-1.14 | |
| | | | | (m, 3H). | |
| 90 | 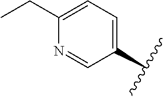 | ⁿPr— | 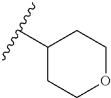 [SMILES: *C1CCOCC1] | (400 MHz, CDCl₃): δ 8.50-8.49 (m, 1H), 7.76 (s, 1H), 7.60-7.58 (m, 1H), 7.27-7.25 (m, 1H), 4.93- 4.89 (m, 1H), 4.51-4.46 (m, 1H), 4.11-4.08 (m, | 2.46 min./ Method B |
| | | | | 2H), 3.89-3.84 (m, 1H), | |
| | | | | 3.62-3.56 (m, 3H), 3.44- | |
| | | | | 3.32 (m, 1H), 2.93-2.85 | |
| | | | | (m, 3H), 2.13-2.04 (m, | |
| | | | | 2H), 1.95-1.85 (m, 2H), | |
| | | | | 1.61-1.54 (m, 2 H), 1.35- | |
| | | | | 1.28 (m, 3H), 0.92-0.89 | |
| | | | | (m, 3H). | |
| 91 | 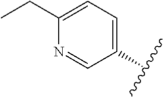 | ⁿPr— | 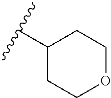 [SMILES: *C1CCOCC1] | (400 MHz, CDCl₃): δ 8.51-8.50 (m, 1H), 7.77 (s, 1H), 7.61-7.58 (m, 1H), 7.28-7.26 (m, 1H), 4.94- 4.90 (m, 1H), 4.52-4.47 (m, 1H), 4.12-4.08 (m, | 1.83 min./ Method B |
| | | | | 2H), 3.89-3.84 (m, 1H), | |
| | | | | 3.62-3.57 (m, 2H), 3.43- | |
| | | | | 3.36 (m, 2H), 2.92-2.85 | |
| | | | | (m, 3H), 2.13-2.07 (m, | |
| | | | | 2H), 1.96-1.92 (m, 2H), | |
| | | | | 1.62-1.55 (m, 2H), 1.36- | |
| | | | | 1.32 (m, 3H), 0.93-0.89 | |
| | | | | (m, 3H). | |
| 92 | 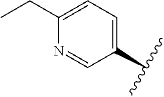 | ^{iso}Bu— | 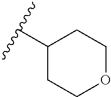 [SMILES: *C1CCOCC1] | (400 MHz, DMSO-d₆): δ 0.90-0.94 (m, 6H), 1.34 (t, J = 7.8 Hz, 3H), 1.91-1.98 (m, 3H), 2.04-2.16 (m, 2H), 2.74-2.79 (m, 1H), 2.86-2.92 (m, 2H), 3.21- | 3.09 min./ Method D |
| | | | | 3.27 (m, 1H), 3.31-3.39 | |
| | | | | (m, 1H), 3.56-3.64 (m, | |
| | | | | 2H), 3.92-3.96 (m, 1H), | |
| | | | | 4.08-4.15 (m, 2H), 4.46- | |
| | | | | 4.51 (m, 1H), 4.91-4.95 | |
| | | | | (m, 1H), 7.26-7.27 (m, | |
| | | | | 1H), 7.55-7.58 (m, 1H), | |
| | | | | 7.78 (s, 1H), 8.48-8.49 (m, | |
| | | | | 1H). | |
| 93 | 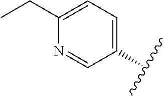 | ^{iso}Bu— | 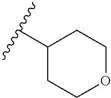 [SMILES: *C1CCOCC1] | (400 MHz, DMSO-d₆): δ 0.89-0.93 (m, 6H), 1.34 (t, J = 7.6 Hz, 3H), 1.91-1.98 (m, 3H), 2.01-2.18 (m, 2H), 2.74-2.79 (m, 1H), 2.85-2.91 (m, 2H), 3.21- | 2.09 min./ Method D |
| | | | | 3.27 (m, 1H), 3.31-3.39 | |
| | | | | (m, 1H), 3.57-3.63 (m, | |
| | | | | 2H), 3.91-3.96 (m, 1H), | |
| | | | | 4.09-4.13 (m, 2H), 4.46- | |
| | | | | 4.51 (m, 1H), 4.91-4.95 | |
| | | | | (m, 1H), 7.25-7.27 (m, | |
| | | | | 1H), 7.55-7.57 (m, 1H), | |
| | | | | 7.78 (s, 1H), 8.47-8.48 (m, | |
| | | | | 1H). | |
| 94 | 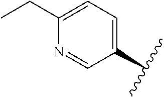 | Et— | 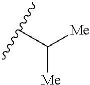 [SMILES: *C(C)C] | (400 MHz, CDCl₃): δ 8.56 (s, 1H), 7.93 (dd, J = 8.4, 2.4 Hz, 1H), 7.65 (s, 1H), 7.45 (d, J = 8.4 Hz, 1H), 5.15 (t, J = 8.4 Hz, 1H), 4.55 (t, J = 9.2 Hz, | 5.63 min./ Method E |
| | | | | 1H), 3.86 (t, J = 8.0 Hz, | |
| | | | | 1H), 3.55-3.49 (m, 2H), | |
| | | | | 3.07-3.02 (m, 1H), 2.88 (q, | |
| | | | | J = 15.2, 8.0 Hz, 2H), 1.40 | |
| | | | | (s, 3H), 1.38 (s, 3H), 1.33 | |
| | | | | (t, J = 7.2 Hz, 3H), 1.17 (t, | |
| | | | | J = 7.2 Hz, 3H). | |
| 95 | 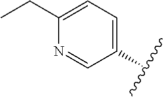 | Et— | 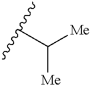 [SMILES: *C(C)C] | (400 MHz, CD₃OD): δ 8.56 (s, 1H), 7.93 (dd, J = 8.0, 2.4 Hz, 1H), 7.65 (s, 1H), 7.45 (d, J = 8.0 Hz, 1H), 5.15 (t, J = 8.4 Hz, 1H), 4.55 (t, J = 9.6 Hz, | 3.83 min./ Method E |
| | | | | 1H), 3.85 (t, J = 8.0 Hz, | |
| | | | | 1H), 3.55-3.49 (m, 2H), | |
| | | | | 3.07-3.02 (m, 1H), 2.88 (q, | |
| | | | | J = 15.6, 8.0 Hz, 2H), 1.40 | |
| | | | | (s, 3H), 1.38 (s, 3H), 1.33 | |
| | | | | (t, J = 7.2 Hz, 3H), 1.17 (t, | |
| | | | | J = 7.2 Hz, 3H). | |
| 96 | 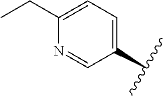 | ⁿPr— | 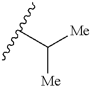 [SMILES: *C(C)C] | (400 MHz, CDCl₃): δ 0.90 (t, J = 7.6 Hz, 3H), 1.30- 1.38 (m, 9H), 1.50-1.63 (m, 2H), 2.84-2.87 (m, 2H), 2.80-3.01 (m, 1H), 3.30-3.53 (m, 2H), 3.84- | 3.46 min./ Method F |
| | | | | 3.91 (m, 1H), 4.52-4.59 | |
| | | | | (m, 1H), 5.11-5.16 (m, | |
| | | | | 1H), 7.43 (d, J = 8.0 Hz, | |
| | | | | 1H), 7.64 (s, 1H), 7.90 (d, | |
| | | | | J = 8.4 Hz, 1H), 8.53 (s, | |
| | | | | 1H). | |
| 97 | 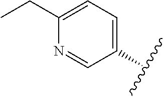 | ⁿPr— | 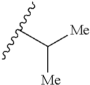 [SMILES: *C(C)C] | (400 MHz, CDCl₃): δ 0.90 (t, J = 7.6 Hz, 3H), 1.30- 1.38 (m, 9H), 1.50-1.63 (m, 2H), 2.84-2.87 (m, 2H), 2.80-3.01 (m, 1H), 3.30-3.53 (m, 2H), 3.84- | 2.46 min./ Method F |
| | | | | 3.91 (m, 1H), 4.52-4.59 | |
| | | | | (m, 1H), 5.11-5.16 (m, | |
| | | | | 1H), 7.43 (d, J = 8.0 Hz, | |
| | | | | 1H), 7.64 (s, 1H), 7.90 (d, | |
| | | | | J = 8.4 Hz, 1H), 8.53 (s, | |
| | | | | 1H). | |
| 98 | 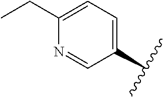 | ^{iso}Bu— | 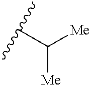 [SMILES: *C(C)C] | (400 MHz, CD₃OD): δ 0.87-0.92 (m, 6H), 1.29- 1.33 (m, 3H), 1.36-1.38 (m, 6H), 1.89-1.95 (m, 1H), 2.83-2.89 (m, 3H), 3.20- 3.23 (m, 1H), 3.44-3.51 (m, | 4.10 min./ Method A |
| | | | | 1H), 3.90-3.95 (m, 1H), | |
| | | | | 3.52-3.57 (m, 1H), 5.11- | |
| | | | | 5.15 (m, 1H), 7.42-7.44 | |
| | | | | (m, 1H), 7.64 (s, 1H), | |
| | | | | 7.88-7.90 (m, 1H), 8.53 (s, | |
| | | | | 1H). | |
| 99 | 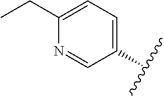 | ^{iso}Bu— | 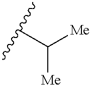 [SMILES: *C(C)C] | (400 MHz, CD₃OD): δ 0.87-0.92 (m, 6H), 1.29- 1.33 (m, 3H), 1.36-1.38 (m, 6H), 1.89-1.95 (m, 1H), 2.83-2.89 (m, 3H), 3.20-3.23 (m, 1H), 3.44- | 6.16 min./ Method A |
| | | | | 3.51 (m, 1H), 3.90-3.95 | |
| | | | | (m, 1H), 3.52-3.57 (m, | |
| | | | | 1H), 5.11-5.15 (m, 1H), | |
| | | | | 7.42-7.44 (m, 1H), 7.64 (s, | |
| | | | | 1H), 7.88-7.90 (m, 1H), | |
| | | | | 8.53 (s, 1H). | |

### Example 100

### 1-(4-Hydroxybutyl)-8-isopropyl-2,3-dihydro-1H,5H-diimidazo[2,1-c:5′,1′-f][1,2,4]triazin-5-one

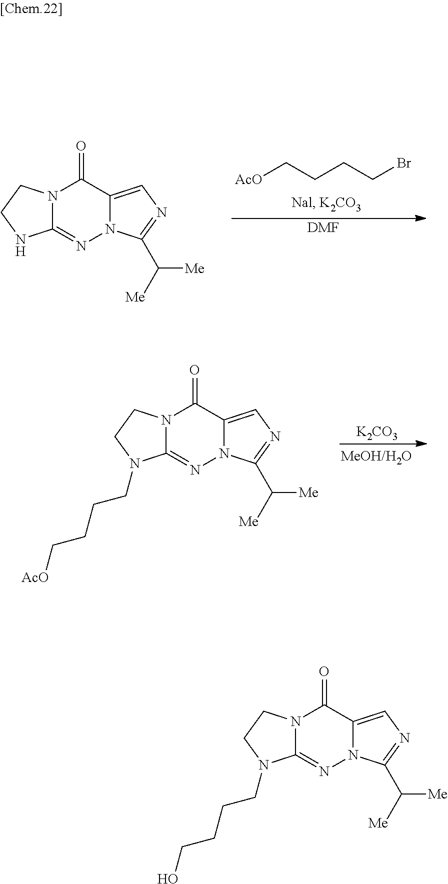

The reaction mixture of 4-[5-oxo-8-(propan-2-yl)-2,3-dihydro-1H,5H-diimidazo[2,1-c:5′,1′-f][1,2,4]triazin-1-yl]butyl acetate was diluted with MeOH (5 mL) and water (5 mL). The mixture was stirred at 25° C. for 2 h. The residue was diluted with water (10 mL) and extracted with DCM (10 mL×3). The combined organic layers was concentrated to dryness to give the crude, which was purified by prep-HPLC (0.1% NH₃.H₂O as additive) to give the title compound (30 mg, 23% yield of 2 steps) as a gray solid. ¹H NMR (400 MHz, CDCl₃): δ 7.73 (s, 1H), 4.13-4.08 (m, 2H), 3.76-3.73 (m, 2H), 3.70-3.66 (m, 2H), 3.44-3.40 (m, 3H), 1.81-1.75 (m, 2H), 1.69-1.65 (m, 2H), 1.38 (d, J=7.2 Hz, 6H).

### 4-[5-Oxo-8-(propan-2-yl)-2,3-dihydro-1H,5H-diimidazo[2,1-c:5′,1′-f][1,2,4]triazin-1-yl]butyl acetate

A mixture of 8-(propan-2-yl)-2,3-dihydro-1H,5H-diimidazo[2,1-c:5′,1′-f][1,2,4]triazin-5-one (100 mg, 0.456 mmol), 4-bromobutyl acetate (178 mg, 0.91 mmol), trace of NaI and K₂CO₃ (126 g, 0.91 mmol) in dry DMF (2 mL) was heated to 50° C. for 16 h. After cooled to room temperature, the reaction solution was used to next step without further purification.

### Example 101

### 1-(5-Hydroxypentyl)-9-isopropyl-1,2,3,4-tetrahydro-6H-imidazo[5,1-f]pyrimido[2,1-c][1,2,4]triazin-6-one

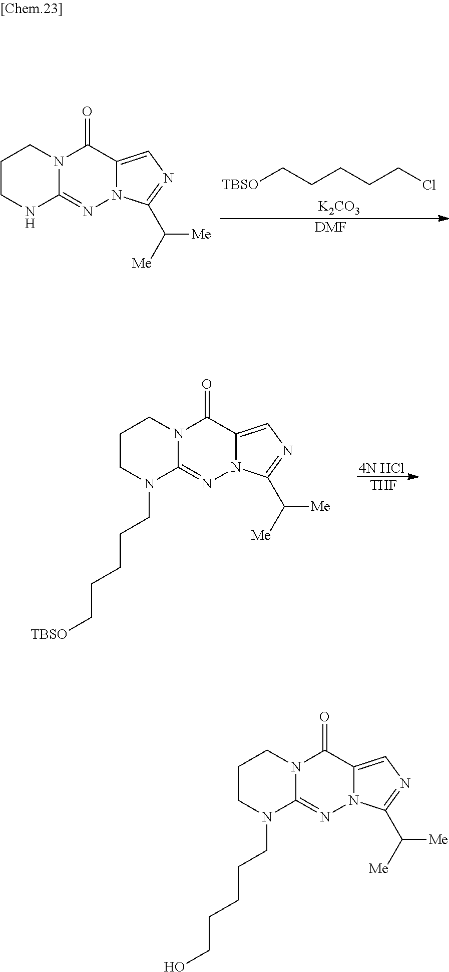

A solution of 1-(5-{[tert-Butyl(dimethyl)silyl]oxy}pentyl)-9-(propan-2-yl)-1,2,3,4-tetrahydro-6H-imidazo[5,1-f]pyrimido[2,1-c][1,2,4]triazin-6-one (127 mg, 0.29 mmol) in THF (1 mL) and 4 N HCl (1 mL) was stirred at 30° C. for 16 h. The mixture concentrated to dryness and basified to pH=9 with Et₃N. The residue was purified by prep-HPLC (0.1% NH₃.H₂O as additive) to give the title compound (50 mg, 53%). ¹H NMR (400 MHz, CDCl₃): δ 7.68 (s, 1H), 4.05-3.90 (m, 2H), 3.75-3.57 (m, 2H), 3.57-3.26 (m, 5H), 2.15-1.90 (m, 3H), 1.80-1.55 (m, 4H), 1.55-1.25 (m, 8H).

### 1-(5-{[tert-Butyl(dimethyl)silyl]oxy}pentyl)-9-(propan-2-yl)-1,2,3,4-tetrahydro-6H-imidazo[5,1-f]pyrimido[2,1-c][1,2,4]triazin-6-one

A mixture of 9-(propan-2-yl)-1,2,3,4-tetrahydro-6H-imidazo[5,1-f]pyrimido[2,1-c][1,2,4]triazin-6-one (100 mg, 0.43 mmol), tert-butyl[(5-chloropentyl)oxy]dimethylsilane (154 mg, 0.65 mmol) and NaH (60% in mineral oil, 26 mg, 0.65 mmol) in anhydrous THF (2 mL) was heated to reflux for 16 h. The mixture quenched with water (20 mL) and extracted with EtOAc (15 mL×3). The combined organic layers were washed with brine, dried over Na₂SO₄ and concentrated to dryness. The residue was purified by prep-TLC (PE/EtOAc=1/1) to give the title compound (127 mg, 68%). ¹H NMR (400 MHz, CDCl₃): δ 7.71 (s, 1H), 4.01-3.98 (m, 2H), 3.65-3.61 (m, 2H), 3.52-3.48 (m, 2H), 3.42-3.35 (m, 3H), 2.10-2.05 (m, 2H), 1.71-1.68 (m, 6H), 1.60-1.55 (m, 2H), 1.46-1.37 (m, 8H), 0.88 (s, 9H).

The compounds of Examples 102 to 109 were synthesized in a similar manner to Example 101.

TABLE 10

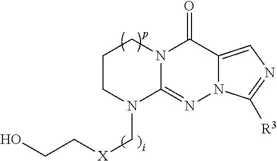

| No. | p | i | X | R³ | ¹H NMR |
|---|---|---|---|---|---|
| 102 | 0 | 2 | CH₂ | 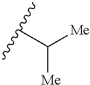 [SMILES: *C(C)C] | (400 MHz, CDCl₃): δ 7.81 (s, 1H), 4.25-4.05 (m, 2H), 3.85-3.65 (m, 4H), 3.60-3.47 (m, 1H), 3.47-3.34 (m, 2H), 1.82-1.60 (m, 4H), 1.60- 1.34 (m, 8H). |
| 103 | 0 | 3 | CH₂ | 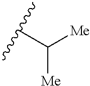 [SMILES: *C(C)C] | (400 MHz, CDCl₃): δ 7.69 (s, 1H), 4.09-4.05 (m, 2H), 3.66-3.62 (m, 4H), 3.42-3.32 (m, 3H), 1.92 (br s, 1H), 1.70-1.55 (m, 4H), 1.48-1.40 (m, 4H), 1.35 (d, J = 7.2 Hz, 6H). |
| 104 | 0 | 2 | O | 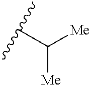 [SMILES: *C(C)C] | (400 MHz, CDCl₃): δ 7.74 (s, 1H), 4.18-4.02 (m, 2H), 3.92-3.72 (m, 6H), 3.72-3.55 (m, 4H), 3.50-3.33 (m, 1H), 2.13 (brs, 1H), 1.37 (d, J = 6.8 Hz, 6H). |
| 105 | 1 | 1 | CH₂ | 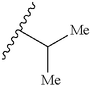 [SMILES: *C(C)C] | (400 MHz, CDCl₃): δ 7.72 (s, 1H), 4.03-4.00 (m, 2H), 3.76-3.72 (m, 2H), 3.59-3.55 (m, 2H), 3.60-3.36 (m, 3H), 2.12-2.06 (m, 2H), 1.87- 1.80 (m, 2H), 1.73-1.62 (m, 3H), |
| | | | | | 1.39 (d, J = 7.2 Hz, 6H). |
| 106 | 1 | 3 | CH₂ | 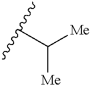 [SMILES: *C(C)C] | (400 MHz, CDCl₃): δ 7.70 (s, 1H), 4.01-3.98 (m, 2H), 3.67-3.64 (m, 2H), 3.50-3.40 (m, 5H), 2.08- 2.05 (m, 2H), 1.74-1.71 (m, 2H), 1.60-1.58 (m, 2H), 1.42 (br s, |
| | | | | | 4H), 1.37 (d, J = 6.8 Hz, 6H). |
| 107 | 1 | 2 | O | 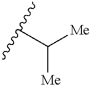 [SMILES: *C(C)C] | (400 MHz, CDCl₃): δ 7.71 (s, 1H), 4.05-3.95 (m, 2H), 3.88-3.80 (m, 2H), 3.80-3.67 (m, 4H), 3.65-3.50 (m, 4H), 3.44-3.28 (m, 1H), 2.13- 2.02 (m, 2H), 1.98 (brs, 1H), 1.36 |
| | | | | | (d, J = 6.4 Hz, 6H). |
| 108 | 0 | 2 | CH₂ | 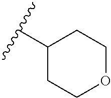 [SMILES: *C1CCOCC1] | (400 MHz, CDCl₃): δ 7.74 (s, 1H), 4.13-4.09 (m, 4H), 3.71-3.58 (m, 6H), 3.41-3.31 (m, 3H), 2.20-2.09 (m, 2H), 1.94-1.90 (m, 2H), 1.75- 1.63 (m, 5H), 1.54-1.47 (m, 2H). |
| 109 | 1 | 2 | CH₂ | 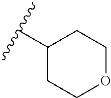 [SMILES: *C1CCOCC1] | (400 MHz, CDCl₃): δ 7.76 (s, 1H), 4.13-3.10 (m, 2H), 4.05-4.02 (m, 2H), 3.71-3.67 (m, 2H), 3.61-3.35 (m, 7H), 2.28-2.20 (m, 2H), 2.12-2.10 (m, 2H), 1.93-1.90 (m, 2H), 1.79-1.76 (m, 2H), 1.70-1.61 (m, 2H), 1.51-1.46 (m, 2H). |

### Example 110

### 1-(4,5-Dihydroxypentyl)-8-isopropyl-2,3-dihydro-1H,5H-diimidazo[2,1-c:5′,1′-f][1,2,4]triazin-5-one

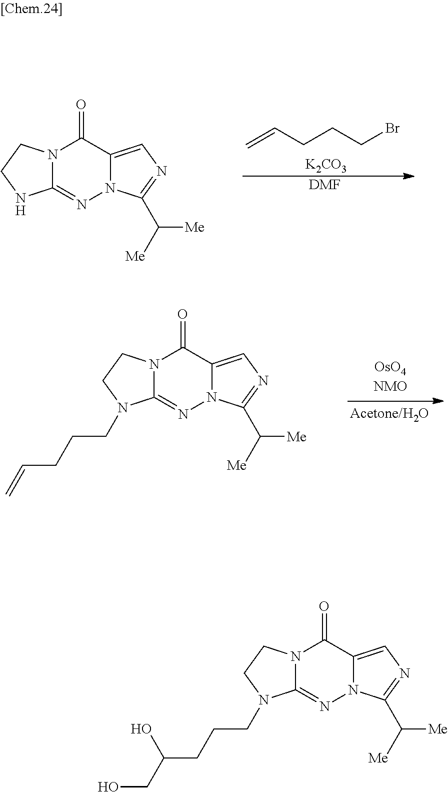

A solution of 1-(pent-4-en-1-yl)-8-(propan-2-yl)-2,3-dihydro-1H,5H-diimidazo[2,1-c:5′,1′-f][1,2,4]triazin-5-one (120 mg, 0.416 mmol), N-methyl-morpholine N-oxide (122 mg, 1.04 mmol), catalytic amount of OsO₄ in water/acetone (1/4) (5 mL) was stirred at 30° C. for 16 h. The mixture was concentrated to dryness. The residue was purified by prep-HPLC (0.1% NH₃. H₂O as additive) to afford the title compound as white solid (75 mg, 56%). ¹H NMR (400 MHz, CDCl₃): δ 7.68 (s, 1H), 4.17-4.00 (m, 2H), 3.86-3.60 (m, 4H), 3.55-3.30 (m, 4H), 2.40 (br, 2H), 1.95-1.65 (m, 2H), 1.60-1.45 (m, 2H), 1.33 (d, J=6.8 Hz, 6H).

### 1-(Pent-4-en-1-yl)-8-(propan-2-yl)-2,3-dihydro-1H,5H-diimidazo[2,1-c:5′,1′-f][1,2,4]triazin-5-one

A mixture of 8-(propan-2-yl)-2,3-dihydro-1H,5H-diimidazo[2,1-c:5′,1′-f][1,2,4]triazin-5-one (100 mg, 0.457 mmol), 5-bromo-1-pentene (136 mg, 0.913 mmol) and K₂CO₃ (126 mg, 0.913 mmol) in anhydrous DMF (2 mL) was stirred at 30° C. for 16 h. The mixture quenched with water (15 mL) and extracted with EtOAc (15 mL×3). The combined organic layers were washed with brine, dried over Na₂SO₄ and concentrated to dryness. The residue was purified by prep-TLC (PE/EtOAc=1/2) to give the title compound (120 mg, 92%) as white solid. ¹H NMR (400 MHz, CDCl₃): δ 7.77 (s, 1H), 5.94-5.80 (m, 1H), 5.15-5.02 (m, 2H), 4.17-4.07 (m, 2H), 3.75-3.65 (m, 2H), 3.54-3.35 (m, 3H), 2.25-2.15 (m, 2H), 1.86-1.75 (m, 2H), 1.42 (d, J=6.8 Hz, 6H).

### Example 111

### 1-(4,5-Dihydroxypentyl)-9-isopropyl-1,2,3,4-tetrahydro-6H-imidazo[51-f]pyrimido[2,1-c][1,2,4]triazin-6-one

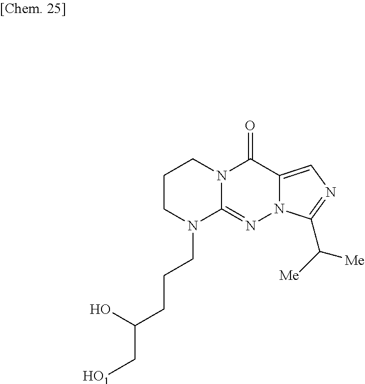

The titled compound was synthesized in a similar manner to Example 110.

¹H NMR (400 MHz, CDCl₃): δ 7.67 (s, 1H), 4.05-3.90 (m, 2H), 3.80-3.63 (m, 2H), 3.58-3.27 (m, 6H), 2.80 (brs, 2H), 2.13-2.00 (m, 2H), 2.00-1.70 (m, 2H), 1.56-1.43 (m, 2H), 1.34 (d, J=6.8 Hz, 6H).

### Example 112

### 9-Isopropyl-1-[(1-methyl-4-piperidinyl)methyl]-1,2,3,4-tetrahydro-6H-imidazo[5,1-f]pyrimido[2,1-c][1,2,4]triazin-6-one

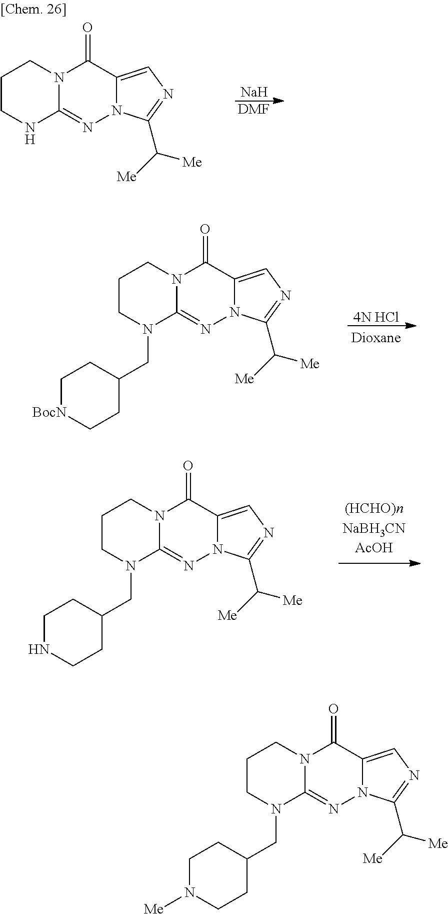

A solution of compound 9-isopropyl-1-(4-piperidinylmethyl)-1,2,3,4-tetrahydro-6H-imidazo[5,1-f]pyrimido[2,1-c][1,2,4]triazin-6-one (115 mg, 0.35 mmol) and (HCHO)n (52 mg, 1.75 mmol) in AcOH (2 mL) was stirred at 25° C. for 1 h. Then NaBH₃CN (110 mg, 1.75 mmol) was added. The mixture was stirred at 25° C. for 1 h. The solvent was removed under reduced pressure and basified to pH=10 with aq. K₂CO₃. The water phase was concentrated to dryness and the residue was purified by prep-HPLC (0.1% NH₃.H₂O as additive) to give the title compound (58 mg, 48%). ¹H NMR (400 MHz, CD₃OD): δ 7.57 (s, 1H), 4.00-3.97 (m, 2H), 3.50-3.40 (m, 5H), 2.92-2.89 (m, 2H), 2.26 (s, 3H), 2.10-1.95 (m, 5H), 1.79-1.76 (m, 2H), 1.45-1.34 (m, 8H).

### tert-Butyl 4-{[6-oxo-9-(propan-2-yl)-3,4-dihydro-6H-imidazo[5,1-f]pyrimido[2,1-c][1,2,4]triazin-1(2H)-yl]methyl}piperidine-1-carboxylate

A solution of 9-(propan-2-yl)-1,2,3,4-tetrahydro-6H-imidazo[5,1-f]pyrimido[2,1-c][1,2,4]triazin-6-one (300 mg, 1.29 mmol) in anhydrous DMF (2 mL) was added NaH (60% in mineral oil, 77 mg, 1.94 mmol) at 25° C. Then the mixture was stirred at 70° C. for 1 hour. After cooled to room temperature, tert-butyl 4-(bromomethyl)piperidine-1-carboxylate (538 mg, 1.94 mmol) was added. The mixture was heated to 80° C. for 16 hrs. The resulting mixture quenched with water (20 mL) and extracted with EtOAc (15 mL×3). The combined organic layers were washed with brine, dried over Na₂SO₄ and concentrated to dryness. The residue was purified by silica gel chromatography (elution with PE/EtOAc=1/1) to give the title compound (501 mg, 90%). ¹H NMR (400 MHz, CDCl₃): δ 7.71 (s, 1H), 4.20-4.10 (m, 1H), 4.05-3.99 (m, 2H), 3.45-3.33 (m, 5H), 2.75-2.65 (m, 2H), 2.15-2.06 (m, 3H), 1.71-1.68 (m, 3H), 1.45 (s, 9H), 1.37 (d, J=6.8 Hz, 6H), 1.30-1.15 (m, 2H).

### 9-Isopropyl-1-(4-piperidinylmethyl)-1,2,3,4-tetrahydro-6H-imidazo[5,1-f]pyrimido[2,1-c][1,2,4]triazin-6-one

A solution of tert-Butyl 4-{[6-oxo-9-(propan-2-yl)-3,4-dihydro-6H-imidazo[5,1-f]pyrimido[2,1-c][1,2,4]triazin-1(2H)-yl]methyl}piperidine-1-carboxylate in HCl/dioxane (10 mL, 4 N) was stirred at 25° C. for 16 h. The solvent was removed under reduced pressure and basified to pH=10 with Et₃N. The residue was purified by prep-HPLC (0.1% NH₃.H₂O as additive) to give the title compound (150 mg, 35%). ¹H NMR (400 MHz, CD₃OD): δ 7.56 (s, 1H), 4.00-3.98 (m, 2H), 3.50-3.41 (m, 5H), 3.25-3.18 (m, 2H), 2.74-2.68 (m, 2H), 2.25-2.05 (m, 3H), 1.87-1.83 (m, 2H), 1.42-1.35 (m, 8H).

### Example 113

### 9-Isopropyl-1-[2-(4-piperidinyl)ethyl]-1,2,3,4-tetrahydro-6H-imidazo[5,1-f]pyrimido[2,1-c][1,2,4]triazin-6-one

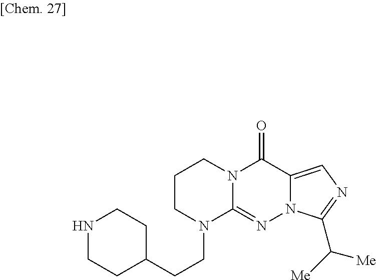 [SMILES: CC(C)c1ncc2c(=O)n3c(nn12)N(CCC1CCNCC1)CCC3]

The titled compound was synthesized in a similar manner to Example 112.

¹H NMR (400 MHz, CD₃OD): δ 7.56 (s, 1H), 3.97-3.95 (m, 2H), 3.60-3.57 (m, 2H), 3.47-3.42 (m, 3H), 3.30-3.10 (m, 2H), 2.75-2.65 (m, 2H), 2.08-2.05 (m, 2H), 1.86-1.83 (m, 2H), 1.75-1.65 (m, 2H), 1.60-1.50 (m, 1H), 1.40-1.25 (m, 8H).

### Example 114

### 9-Isopropyl-1-[2-(1-methyl-4-piperidinyl)ethyl]-1,2,3,4-tetrahydro-6H-imidazo[5,1-f]pyrimido[2,1-c][1,2,4]triazin-6-one

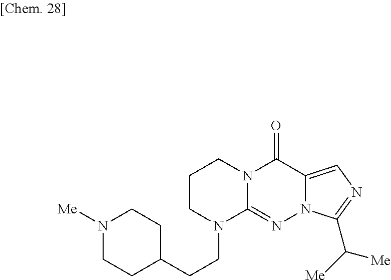 [SMILES: CC(C)c1ncc2c(=O)n3c(nn12)N(CCC1CCN(C)CC1)CCC3]

The titled compound was synthesized in a similar manner to Example 112.

¹H NMR (400 MHz, CD₃OD): δ 7.59 (s, 1H), 4.01-3.98 (m, 2H), 3.63-3.61 (m, 2H), 3.50-3.47 (m, 3H), 2.90-2.88 (m, 2H), 2.28 (s, 3H), 2.11-2.04 (m, 4H), 1.84-1.82 (m, 2H), 1.69-1.74 (m, 2H), 1.38-1.37 (m, 9H).

### Example 115

### 3-(2-(9-Isopropyl-6-oxo-2,3,4,6-tetrahydro-1H-imidazo[1,5-f]pyrimido[2,1-c][1,2,4]triazin-1-yl)ethyl)benzonitrile

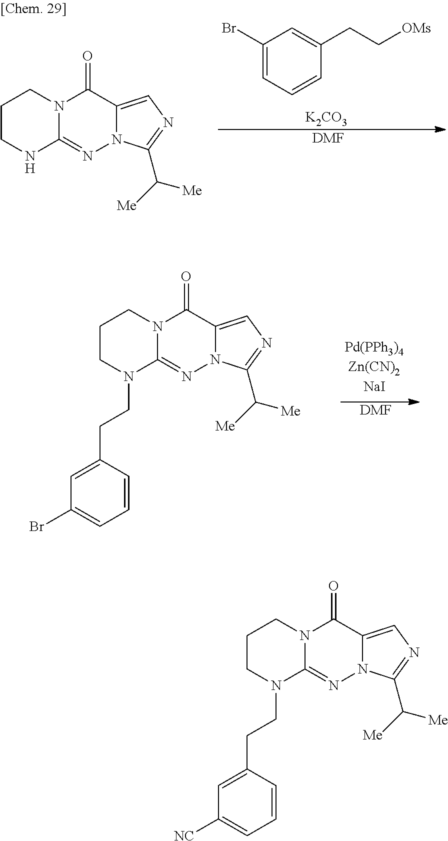

To a solution of 1-(3-bromophenethyl)-9-isopropyl-3,4-dihydro-1H-imidazo[1,5-f]pyrimido[2,1-c][1,2,4]triazin-6(2H)-one (55 mg, 0.13 mmol) in DMF (2 mL) was added Zn(CN)₂ (30 mg, 0.26 mmol), NaI (20 mg, 0.13 mmol), Pd(PPh₃)₄(30 mg, 0.03 mmol). The reaction mixture was stirred at 120° C. for 3 h under microwave and purified by prep-HPLC (MeCN and H₂O with 0.01% NH₃.H₂O as mobile phase) to give the title compound as a white solid (20 mg, 43%). LC-MS (m/z)=363 [M+H]⁺. ¹H NMR (400 MHz, DMSO-d₆): δ 1.31 (d, J=6.8 Hz, 6H), 1.91-1.94 (m, 2H), 3.01-3.05 (m, 2H), 3.31-3.36 (m, 3H), 3.66-3.69 (m, 2H), 3.82-3.84 (m, 2H), 7.50-7.54 (m, 2H), 7.62-7.69 (m, 2H), 7.79 (s, 1H).

### 1-(3-Bromophenethyl)-9-isopropyl-3,4-dihydro-1H-imidazo[1,5-f]pyrimido[2,1-c][1,2,4]triazin-6(2H)-one

To a solution of 3-bromophenethyl methanesulfonate (550 mg, 2 mmol) and 9-isopropyl-3,4-dihydro-1H-imidazo[1,5-f]pyrimido[2,1-c][1,2,4]triazin-6(2H)-one (116 mg, 0.5 mmol) in DMF (3 mL) was added Cs₂CO₃ (975 mg, 3 mmol). The reaction mixture was stirred at 50° C. for 5 h and purified by prep-HPLC (MeCN and H₂O with 0.01% NH₃.H₂O as mobile phase) to give the title compound as a white solid (60 mg, 29%). LC-MS (m/z)=416 [M+H]⁺. ¹H NMR (400 MHz, CDCl₃): δ 1.51 (d, J=7.2 Hz, 6H), 2.03-2.06 (m, 2H), 2.99-3.08 (m, 4H), 3.58-3.59 (m, 1H), 3.72-3.75 (m, 2H), 4.00-4.03 (m, 2H), 7.14-7.24 (m, 2H), 7.40-7.42 (m, 2H), 7.95 (s, 1H).

### Example 116

### 4-{2-[6-Oxo-9-(tetrahydro-2H-pyran-4-yl)-3,4-dihydro-6H-imidazo[5,1-f]pyrimido[2,1-c][1,2,4]triazin-1(2H)-yl]ethyl}benzonitrile

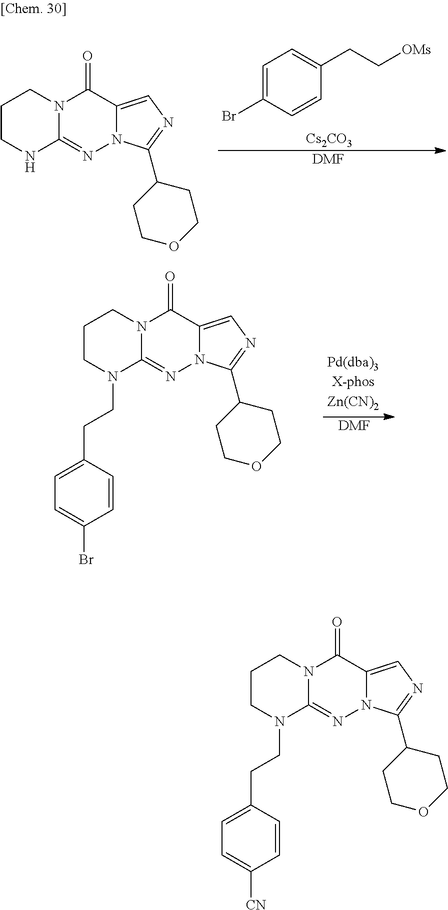

To a solution of 1-(4-bromophenethyl)-9-(tetrahydro-2H-pyran-4-yl)-3,4-dihydro-1H-imidazo[1,5-f]pyrimido[2,1-c][1,2,4]triazin-6(2H)-one (40 mg, 0.09 mmol) in DMF (2 mL) was added Zn(CN)₂ (10.2 mg, 0.09 mmol), Pd₂(dba)₃ (16 mg, 0.018 mmol) and X-phos (8.3 mg, 0.018 mmol). The mixture was stirred at 100° C. overnight. The title compound was purified by reversed phase (0.01% NH₃ in Water and MeCN) as white solid. LC-MS (m/z)=405 [M+H]⁺. ¹H NMR (400 MHz, CDCl₃): δ 1.93-2.05 (m, 4H), 2.16-2.21 (m, 2H), 2.96-3.13 (m, 3H), 3.30-3.33 (m, 2H), 3.55-3.61 (m, 2H), 3.70-3.74 (m, 2H), 3.98-4.01 (m, 2H), 4.11-4.13 (m, 2H), 7.38 (d, J=8.4 Hz, 2H), 7.64 (d, J=8.4 Hz, 2H), 7.75 (s, 1H).

### 1-(4-Bromophenethyl)-9-(tetrahydro-2H-pyran-4-yl)-3,4-dihydro-1H-imidazo[1,5-f]pyrimido[2,1-c][1,2,4]triazin-6(2H)-one

To a solution of 9-(tetrahydro-2H-pyran-4-yl)-3,4-dihydro-1H-imidazo[1,5-f]pyrimido[2,1-c][1,2,4]triazin-6(2H)-one (100 mg, 0.36 mmol) in DMF (3 mL) was added 4-bromophenethyl methanesulfonate (303.3 mg, 1.09 mmol) and Cs₂CO₃ (354.5 mg, 1.09 mmol). The mixture was stirred at 70° C. overnight. The product was purified by column chromatography (EtOAc/PE=4/1) to give the title compound as an oil. LC-MS (m/z)=458 [M+H]⁺. ¹H NMR (400 MHz, CDCl₃): δ 1.94-2.03 (m, 4H), 2.10-2.21 (m, 2H), 2.97-3.01 (m, 2H), 3.29-3.41 (m, 3H), 3.56-3.62 (m, 2H), 3.66-3.70 (m, 2H), 4.11-4.13 (m, 2H), 7.13 (d, J=8.4 Hz, 2H), 7.46 (d, J=8.4 Hz, 1H), 7.75 (s, 1H).

The compounds of Examples 117 to 119 were synthesized in a similar manner to Example 116.

TABLE 11

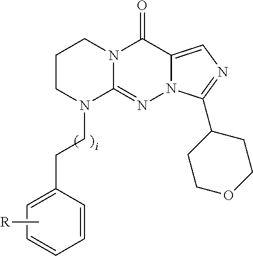

| No. | R— | i | ¹H NMR |
|---|---|---|---|
| 117 | 3-CN | 1 | (400 MHz, CDCl₃): δ 1.94-1.98 (m, 2H), 2.02-2.06 (m, 2H) |
| | | | 2.16-2.20 (m, 2H), 3.10 (t, J = 7.4 Hz, 2H) 3.33-3.37 (m, |
| | | | 3H), 3.57-3.63 (m, 2 H), 3.73 (t, J = 7.6 Hz, 2H), 4.04 (t, |
| | | | J = 5.8 Hz, 2H), 4.12-4.15 (m, 2H), 7.47-7.54 (m, 2H), 7.58- |
| | | | 7.60 (m, 2H), 7.76 (s, 1H). |
| 118 | 3-CN | 2 | (400 MHz, CDCl₃): δ 1.90-1.93 (m, 2H), 2.04-2.19 (m, 6H), |
| | | | 2.76 (t, J = 7.8 Hz, 2H), 3.26-3.32 (m, 1H), 3.43 (t, J = |
| | | | 6.2 Hz, 2H), 3.53-3.59 (m, 4H), 4.01 (t, J = 5.8 Hz, 2H), 4.08- |
| | | | 4.11 (m, 2H), 7.39-7.45 (m, 2H), 7.52-7.55 (m, 2H), 7.74 (s, |
| | | | 1H). |
| 119 | 4-CN | 2 | (400 MHz, CDCl₃): δ 1.89-2.20 (m, 8H), 2.77 (t, J = 7.6 Hz, |
| | | | 2H), 3.25-3.33 (m, 1H), 3.41 (t, J = 6.4 Hz, 2H), 3.52-3.58 |
| | | | (m, 4H), 4.00 (t, J = 6.0 Hz, 2H), 4.07-4.10 (m, 2H), 7.30 |
| | | | (d, J = 8.4 Hz, 2H), 7.60 (d, J = 8.4 Hz, 2H), 7.73 (s, 1H). |

### Example 120

### 9-Isopropyl-1-[3-(2-pyridinyl)propyl]-1,2,3,4-tetrahydro-6H-imidazo[5,1-f]pyrimido[2,1-c][1,2,4]triazin-6-one

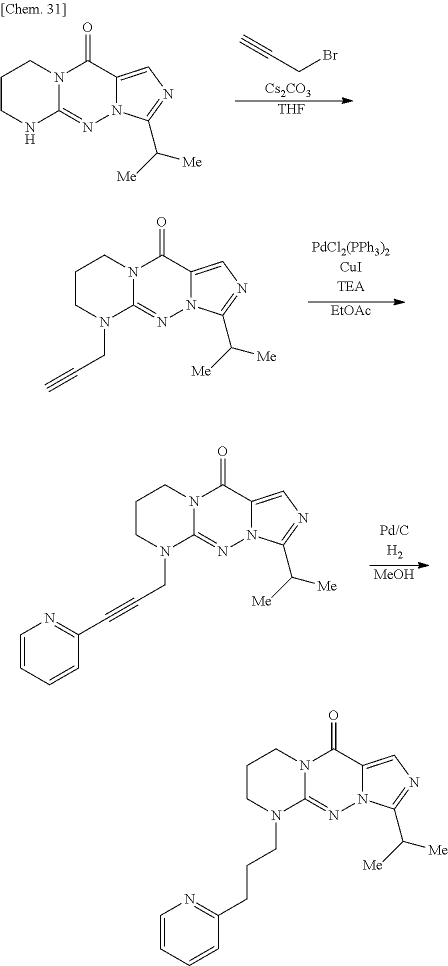

To a solution of 9-isopropyl-1-(3-(pyridin-2-yl)prop-2-ynyl)-3,4-dihydro-1H-imidazo[1,5-f]pyrimido[2,1-c][1,2,4]triazin-6(2H)-one (50 mg, 0.14 mmol) in methanol (2 mL) was added Pd/C (20 mg). The reaction mixture was stirred at room temperature overnight under H₂. Then the mixture was filtered and the filtrate was concentrated and purified by reversed phase (0.01% NH₃ in Water and MeCN) to give the title compound as a white solid (15 mg, 30%). LC-MS (m/z)=353 [M+H]⁺. ¹H NMR (400 MHz, CD₃ OD): δ 1.30 (d, J=7.2 Hz, 6H), 2.03-2.09 (m, 2H), 2.14-2.21 (m, 2H), 2.90 (t, J=7.6 Hz, 2H), 3.25-3.30 (m, 1H), 3.48 (t, J=6.0 Hz, 2H), 3.62 (t, J=7.6 Hz, 2H), 3.95 (t, J=6.0 Hz, 2H), 7.26-7.29 (m, 1H), 7.34-7.36 (m, 1H), 7.57 (s, 1H), 7.73-7.78 (m, 1H), 8.45-8.46 (m, 1H).

### 9-Isopropyl-1-(prop-2-ynyl)-3,4-dihydro-1H-imidazo[1,5-f]pyrimido[2,1-c][1,2,4]triazin-6(2H)-one

To a solution of 9-isopropyl-3,4-dihydro-1H-imidazo[1,5-f]pyrimido[2,1-c][1,2,4]triazin-6(2H)-one (115 mg, 0.5 mmol), 3-bromoprop-1-yne (118 mg, 1.0 mmol), cesium carbonate (325 mg, 1.0 mmol) in THF (2 mL) was stirred at room temperature for 4 h. Then the mixture was washed by water, extracted with ethyl acetate. The combined organic layer was washed with water and brine, dried with sodium sulfate, concentrated to give a residue to obtain the title compound (100 mg, 74%) as a yellow solid. LC-MS (m/z)=272 [M+H]⁺. ¹H NMR (400 MHz, CD₃OD): δ 1.27 (d, J=7.2 Hz, 6H), 1.99-2.06 (m, 2H), 2.60 (t, J=2.4 Hz, 1H), 3.36-3.39 (m, 1H), 3.44-3.47 (m, 2H), 3.89-3.92 (m, 2H), 4.25 (d, J=2.8 Hz, 2H), 7.50 (s, 1H).

### 9-isopropyl-1-(3-(pyridin-2-yl)prop-2-ynyl)-3,4-dihydro-1H-imidazo[1,5-f]pyrimido[2,1-c][1,2,4]triazin-6(2H)-one

A solution of 9-isopropyl-1-(prop-2-ynyl)-3,4-dihydro-1H-imidazo[1,5-f]pyrimido[2,1-c][1,2,4]triazin-6(2H)-one (100 mg, 0.37 mmol), 2-bromopyridine (71 mg, 0.45 mmol), bis(triphenylphosphine)palladium(II) chloride (26 mg, 0.037 mmol), CuI (14 mg, 0.074 mmol), triethylamine (0.1 mL) in ethyl acetate (2 mL) was stirred at room temperature overnight under N₂ protection. Then the mixture was washed by water, extracted with ethyl acetate, and the combined organic layers were washed with water and brine, dried with sodium sulfate, concentrated to give a residue to obtain the title compound (50 mg, 39%) as yellow solid. LC-MS (m/z)=349 [M+H]⁺.

The compounds of Examples 121 and 122 were synthesized in a similar manner to Example 120.

TABLE 12

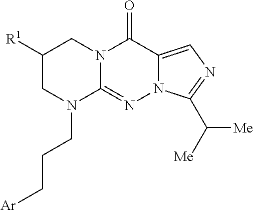

| No. | R¹ | Ar— | ¹H NMR |
|---|---|---|---|
| 121 | Me | 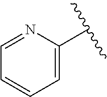 [SMILES: *c1ccccn1] | (400 MHz, CDCl₃): δ 1.10 (d, J = 6.5 Hz, 3H), 1.35 (d, J = 1.5 Hz, 3H), 1.37 (d, J = 1.8 Hz, 3H), 2.07-2.22 (m, 3H), 2.88 (t, J = 7.4 Hz, 2H), 3.04-3.13 (m, 2H), 3.30-3.38 (m, 2H), 3.49-3.57 (m, |
| | | | 1H), 3.62-3.69 (m, 1H), 4.38-4.42 |
| | | | (m, 1H), 7.12-7.15 (m, 2H), 7.56- |
| | | | 7.60 (m, 1H), 7.70 (s, 1H), 8.54 (d, |
| | | | J = 4.6 Hz, 1H). |
| 122 | H | 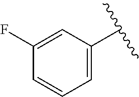 [SMILES: *c1cccc(F)c1] | (400 MHz, CDCl₃): δ 1.36 (d, J = 6.8 Hz, 6H), 2.02-2.10 (m, 4H), 2.71 (t, J = 7.2 Hz, 2H), 3.26- 3.33 (m, 1H), 3.39 (t, J = 6.0 Hz, 2H), 3.53 (t, J = 7.2 Hz, 2H), 3.97 (t, J = 6.0 Hz, 2H), 6.89-6.98 (m, |
| | | | 3H), 7.22-7.26 (m, 1H), 7.71 (s, 1H). |

### Example 123

### 9-Isopropyl-1-(tetrahydro-2H-pyran-4-ylacetyl)-1,2,3,4-tetrahydro-6H-imidazo[5,1-f]pyrimido[2,1-c][1,2,4]triazin-6-one

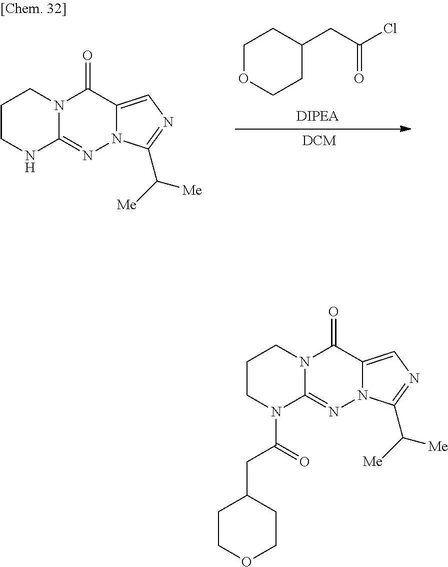 [SMILES: CC(C)c1ncc2c(=O)n3c(nn12)NCCC3 ; O=C(Cl)CC1CCOCC1 ; CC(C)c1ncc2c(=O)n3c(nn12)N(C(=O)CC1CCOCC1)CCC3]

A solution of 2-(tetrahydro-2H-pyran-4-yl)acetyl chloride (112 mg, 0.69 mmol) in dry DCM (5 mL) was dropped into 9-isopropyl-3,4-dihydro-1H-imidazo[1,5-f]pyrimido[2,1-c][1,2,4]triazin-6(2H)-one (20 mg, 0.08 mmol) and DIPEA (179 mg, 1.38 mmol) in dichloromethane (10 mL) at 0° C. The reaction was stirred at 25° C. for 16 h. The reaction was quenched by water (1 mL), and then purified through pre-HPLC to give the title compound. LC-MS (m/z)=360 [M+H]⁺. ¹H NMR (400 MHz, CDCl₃): δ 1.28-1.33 (m, 2H), 1.39-1.40 (m, 6H), 1.67-1.68 (m, 2H), 2.13-2.18 (m, 2H), 2.24-2.26 (m, 1H), 2.78-2.79 (m, 2H), 3.37-3.46 (m, 3H), 3.85-3.86 (m, 2H), 3.93-3.97 (m, 2H), 4.04-4.05 (m, 2H), 7.85 (s, 1H).

### Example 124

### 9-Isopropyl-1-(3-(tetrahydro-2H-pyran-4-yl)propanoyl)-3,4-dihydro-1H-imidazo[1,5-f]pyrimido[2,1-c][1,2,4]triazin-6(2H)-one

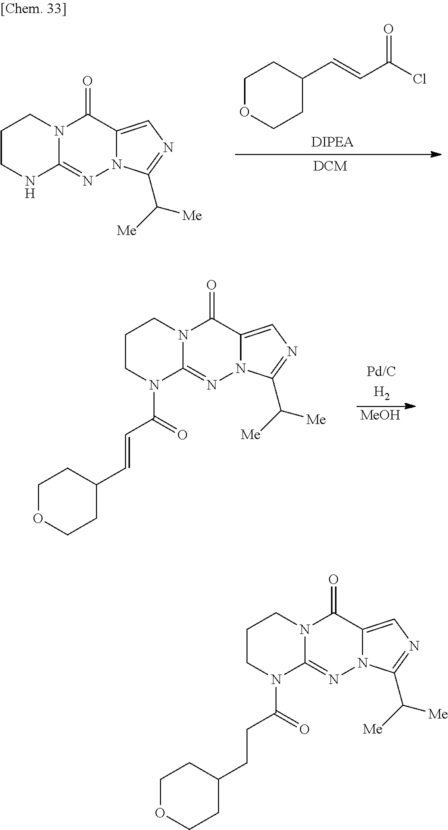

A mixture of (E)-9-isopropyl-1-(3-(tetrahydro-2H-pyran-4-yl)acryloyl)-3,4-dihydro-1H-imidazo[1,5-f]pyrimido[2,1-c][1,2,4]triazin-6(2H)-one (20 mg, 0.054 mmol), Pd—C(4 mg, 10%, w/w) and cesium carbonate (317 mg, 0.96 mmol) in MeOH (3 mL) was stirred at room temperature under H₂ overnight. Purified by reversed phase HPLC to give the title compound (5 mg, 25%) as a solid. LC-MS (m/z)=374 [M+H]⁺. ¹H NMR (400 MHz, CDCl₃): δ 1.25-1.36 (m, 2H), 1.39-1.44 (m, 6H), 1.56-1.60 (m, 3H), 1.68-1.73 (m, 2H), 2.13-2.19 (m, 2H), 2.85-2.89 (m, 2H), 3.31-3.47 (m, 3H), 3.86-3.90 (m, 2H), 3.94-3.98 (m, 2H), 4.04-4.07 (m, 2H), 7.87 (s, 1H).

### (E)-9-Isopropyl-1-(3-(tetrahydro-2H-pyran-4-yl)acryloyl)-3,4-dihydro-1H-imidazo[1,5-f]pyrimido[2,1-c][1,2,4]triazin-6(2H)-one

A mixture of 9-isopropyl-3,4-dihydro-1H-imidazo[1,5-f]pyrimido[2,1-c][1,2,4]triazin-6(2H)-one (100 mg, 0.43 mmol), (E)-3-(tetrahydro-2H-pyran-4-yl)acryloyl chloride (112 mg, 0.64 mmol), DIPEA (166 mg, 1.29 mmol) and 4-dimethylaminopyridine (5 mg, 0.043 mmol) in DCM (2 mL) was stirred at room temperature overnight. Purified by reversed phase HPLC to give the title compound (20 mg, 13%). LC-MS (m/z)=372 [M+H]⁺.

### Example 125: 9-Isopropyl-1-(3-morpholinopropanoyl)-3,4-dihydro-1H-imidazo[1,5-f]pyrimido[2,1-c][1,2,4]triazin-6(2H)-one

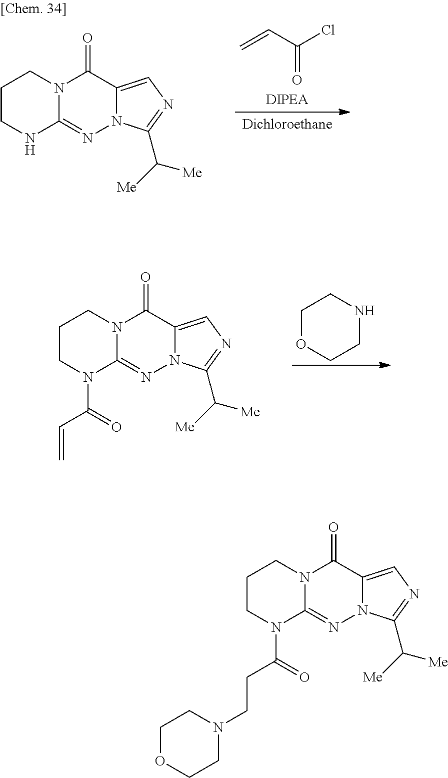

To the mixture of the step 1 (reaction mixture) was added morpholine (150 mg, 1.72 mmol). The resulted reaction mixture was stirred at 0° C. for 2 h. Purified by reversed phase HPLC (H₂O:MeCN=40%) to obtained the title compound (15 mg, 9%). LCMS (m/z)=375 [M+H]⁺. ¹H NMR (400 MHz, CDCl₃): δ 1.37-1.41 (m, 6H), 2.11-2.21 (m, 2H), 2.42-2.45 (m, 4H), 2.72-2.77 (m, 2H), 3.04-3.08 (s, 2H), 3.38-3.50 (m, 1H), 3.64-3.67 (m, 4H), 3.87-3.90 (m, 2H), 4.04-4.07 (m, 2H), 7.86 (s, 1H).

### 1-Acryloyl-9-(propan-2-yl)-1,2,3,4-tetrahydro-6H-imidazo[5,1-f]pyrimido[2,1-c][1,2,4]triazin-6-one

A mixture of 9-isopropyl-3,4-dihydro-1H-imidazo[1,5-f]pyrimido[2,1-c][1,2,4]triazin-6(2H)-one (100 mg, 0.43 mmol), acryloyl chloride (78 mg, 0.86 mmol) and DIPEA (166 mg, 1.29 mmol) in 1,2-dichloroethane (2.0 mL) was stirred at 0° C. for 2 h. The reaction mixture was used directly for next step without further purification. LC-MS (m/z)=288 [M+H]⁺.

### Example 126

### 9-Isopropyl-2-phenyl-3,4-dihydroimidazo[5,1-f][1,3]oxazino[2,3-c][1,2,4]triazin-6(2H)-one

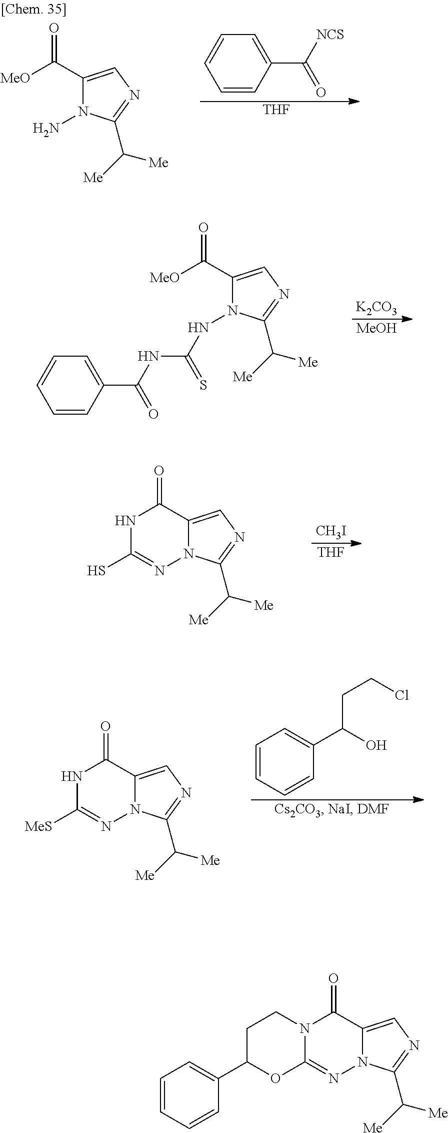

To a mixture of 7-isopropyl-2-(methylthio)imidazo[1,5-f][1,2,4]triazin-4(3H)-one (45 mg, 0.2 mmol), (±)-3-chloro-1-phenylpropan-1-ol (68 mg, 0.4 mmol) and sodium iodide (30 mg, 0.2 mmol) in DMF (3 mL) was added cesium carbonate (55 mg, 0.4 mmol). The reaction was heated to 120° C. under microwave for 2 h. Upon completion, the mixture was quenched into water (10 mL) and extracted with ethyl acetate (40 mL×2). The combined organics were washed with water (20 mL) and brine (20 mL), dried over sodium sulfate, and concentrated in vacuo to give a residue. Purification by flash chromatography on silica (elution with 40:60 ethyl acetate:petroleum ether) gave the title compound (22 mg, 35%) as a white solid. LC-MS (m/z)=311 [M+H]⁺. ¹H NMR (400 MHz, CDCl₃): δ 7.87 (s, 1H), 7.48-7.40 (m, 5H), 5.46 (dd, J=10.0, 2.8 Hz, 1H), 4.39 (dt, J=14.0, 4.4 Hz, 1H), 3.93-3.86 (m, 1H), 3.49 (sept, J=6.8 Hz, 1H), 2.57-2.51 (m, 1H), 2.43-2.36 (m, 1H), 1.40 (t, J=6.8 Hz, 6H).

### Methyl 1-(3-benzoylthioureido)-2-isopropyl-1H-imidazole-5-carboxylate

A mixture of methyl 1-amino-2-isopropyl-1H-imidazole-5-carboxylate (916 mg, 5.0 mmol) and benzoyl isothiocyanate (816 mg, 5.0 mmol) in THF (20 mL) was stirred at room temperature overnight. Upon completion, the solvent was evaporated in vacuo, and the crude product (1.65 g, 95%) was used without further purification. LC-MS (m/z)=347 [M+H]⁺.

### 7-Isopropyl-2-mercaptoimidazo[1,5-f][1,2,4]triazin-4(3H)-one

To a mixture of methyl 1-(3-benzoylthioureido)-2-isopropyl-1H-imidazole-5-carboxylate (1.65 g, 4.7 mmol) in methanol (40 mL) was added potassium carbonate (1.03 g, 7.5 mmol). The reaction was stirred at rt for 1 h, then heated to reflux for 3 h. Upon completion, the mixture was filtered, adjusted pH=7 with acetic acid and concentrated to dryness. The residue was purified by prep-HPLC in 0.01% aqueous ammonia to give the title compound (780 mg, 79%) as a white solid. LC-MS (m/z)=211 [M+H]⁺.

### 7-Isopropyl-2-(methylthio)imidazo[1,5-f][1,2,4]triazin-4(3H)-one

A round bottom flask was charged with 7-isopropyl-2-mercaptoimidazo[1,5-f][1,2,4]triazin-4(3H)-one (780 mg, 3.7 mmol) and THF (30 mL). Methyl iodide (525 mg, 3.7 mmol) was added, and the reaction was stirred at 50° C. for 1 h. The solvent was evaporated under reduced pressure to give an off-white solid. Water (50 mL) and ethyl acetate (200 mL) were added, and the mixture was stirred for 30 minutes. The organic layer was concentrated to dryness to give a residue, which was purified by chromatography on silica (40:60 ethyl acetate:petroleum ether) to give the title compound as a white solid (705 mg, 85%). LC-MS (m/z)=225 [M+H]⁺.

The compounds of Examples 127 to 132 were synthesized in a similar manner to Example 126.

TABLE 13

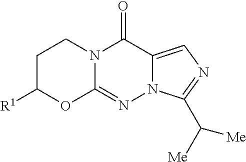

| No. | R¹— | ¹H NMR |
|---|---|---|
| 127 | 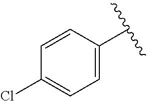 [SMILES: *c1ccc(Cl)cc1] | (400 MHz, CDCl₃): δ 7.88 (s, 1H), 7.44 (d, J = 8.4 Hz, 2H), 7.39 (d, J = 8.0 Hz, 2H), 5.42 (dd, J = 10.0, 2.4 Hz, 1H), 4.44-4.40 (m, 1H), 3.91-3.85 (m, 1H), 3.49 (sept, J = 6.8 Hz, 1H), 2.55-2.51 (m, 1H), 2.37-2.25 (m, 1H), 1.40 (t, J = 6.8 Hz, 6H). |
| 128 | 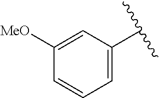 [SMILES: *c1cccc(OC)c1] | (400 MHz, CDCl₃): δ 7.82 (s, 1H), 7.31 (d, J = 8.0 Hz, 1H), 6.86 (dd, J = 8.0, 2.0 Hz, 1H), 6.74 (d, J = 7.6 Hz, 1H), 6.71 (s, 1H), 5.98 (br, 1H), 4.46 (dd, J = 10.8, 4.0 Hz, 1H), 4.34-4.28 (m, 1H), 3.81 (s, 3H), 3.53-3.48 (m, 1H), 2.62-2.55 (m, 1H), 2.34-2.30 (m, 1H), 1.44 (d, J = 7.2 Hz, 3H), 1.41 (d, J = 6.8 Hz, 3H). |
| 129 | 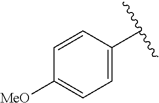 [SMILES: *c1ccc(OC)cc1] | (400 MHz, CDCl₃): δ 7.81 (s, 1H), 7.09 (d, J = 8.8 Hz, 2H), 6.91 (d, J = 8.8 Hz, 2H), 5.98 (br, 1H), 4.47-4.45 (m, 1H), 4.35-4.31 (m, 1H), 3.80 (s, 3H), 3.51 (quint, J = 6.8 Hz, 1H), 2.62-2.50 (m, 1H), 2.30-2.25 (m, 1H), 1.44 (d, J = 7.2 Hz, 3H), 1.41 (d, J = 6.8 Hz, 3H). |
| 130 | 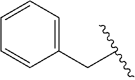 [SMILES: *Cc1ccccc1] | (400 MHz, CDCl₃): δ 7.85 (s, 1H), 7.40-7.27 (m, 5H), 4.62-4.59 (m, 1H), 4.40-4.34 (m, 1H), 3.66-3.59 (m, 1H), 3.53 (sept, J = 7.2 Hz, 1H), 3.34 (dd, J = 14.0, 5.2 Hz, 1H), 3.01 (dd, J = 14.0, 8.0 Hz, 1H), 2.25-2.20 (m, 1H), 1.96-1.85 (m, 1H), 1.40 (d, J = 8.0 Hz, 6H). |
| 131 | 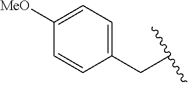 [SMILES: *Cc1ccc(OC)cc1] | (400 MHz, CDCl₃): δ 7.85 (s, 1H), 7.20 (d, J = 8.4 Hz, 2H), 6.91 (d, J = 8.4 Hz, 2H), 4.58-4.55 (m, 1H), 4.36 (dt, J = 14.0, 3.6 Hz, 1H), 3.83 (s, 3H), 3.63 (td, J = 12.4, 4.4 Hz, 1H), 3.54-3.50 (m, 1H), 3.27 (dd, J = 14.0, 5.2 Hz, 1H), 2.97 (dd, J = 14.0, 8.0 Hz, 1H), 2.25-2.20 (m, 1H), 1.94-1.89 (m, 1H), 1.41 (d, J = 6.8 Hz, 6H). |
| 132 | 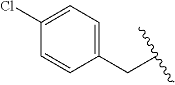 [SMILES: *Cc1ccc(Cl)cc1] | (400 MHz, CDCl₃): δ 7.83 (s, 1H), 7.36 (d, J = 8.4 Hz, 2H), 7.22 (d, J = 8.0 Hz, 2H), 4.61-4.55 (m, 1H), 4.40- 4.35 (m, 1H), 3.64 (td, J = 13.2, 4.8 Hz, 1H), 3.51 (sept, J = 7.2 Hz, 1H), 3.25 (dd, J = 14.0, 5.6 Hz, 1H), 3.02 (dd, J = 14.0, 6.8 Hz, 1H), 2.25-2.20 (m, 1H), 1.97-1.87 (m, 1H), 1.41 (d, J = 6.0 Hz, 6H). |

### Example 133

### 2-(4-chlorophenyl)-8-isopropyl-2H-imidazo[1,5-f]oxazolo[2,3-c][1,2,4]triazin-5(3H)-one

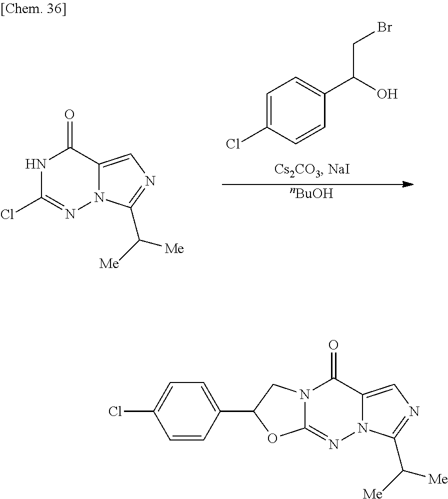

A sealed tube containing 2-bromo-1-(4-chlorophenyl)ethanol (234 mg, 1 mmol), 2-chloro-7-(tetrahydro-2H-pyran-4-yl)imidazo[1,5-f][1,2,4]triazin-4(3H)-one (13 mg, 0.05 mmol), sodium iodide (23 mg, 0.15 mmol), cesium carbonate (49 mg, 0.15 mmol) and ⁿBuOH (4 mL) was heated to 170° C. for 12 h under microwave, and then the crude residue was purified with HPLC to give the title compound (11 mg, 59%) as a white solid. LC-MS (m/z)=373 [M+H]⁺. ¹H NMR (400 MHz, CDCl₃): 7.88 (s, 1H), 7.49-7.47 (m, 2H), 7.39 (d, J=8.0 Hz, 2H), 5.97 (t, J=8.0 Hz, 1H), 4.72-4.67 (m, 1H), 4.17-4.10 (m, 3H), 3.63-3.57 (m, 2H), 3.43-3.37 (m, 1H), 2.21-2.05 (m, 2H), 1.96-1.62 (m, 2H).

### Example 134

### 3-Cyclopentyl-7-phenyl-8,9-dihydro-6H-imidazo[5,1-f]pyrido[2,1-c][1,2,4]triazin-11(7H)-one

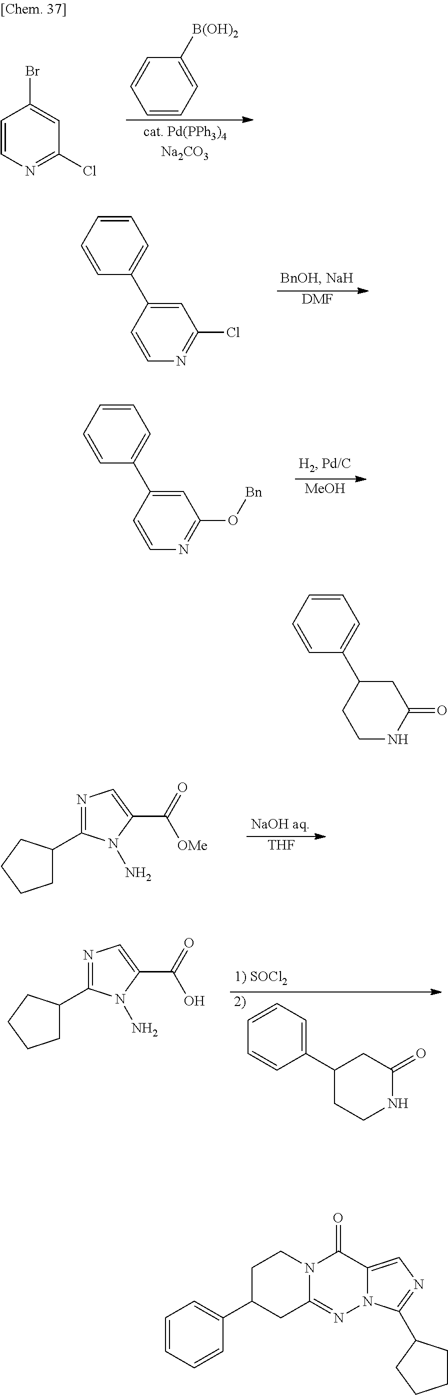

1-Amino-2-cyclopentyl-1H-imidazole-5-carboxylic acid (150 mg, 0.77 mmol) was suspended in thionyl chloride (50 mL) and the resultant mixture heated to reflux until a clear solution was obtained. The thionyl chloride was evaporated in vacuo and the residue was resuspended in dry tetrahydrofuran (8 mL). 4-Phenylpiperidin-2-one (270 mg, 1.54 mmol) was added, and the mixture was stirred at reflux for 2 h. Upon completion, the solvent was removed in vacuo. The residue was purified by flash chromatography to give the title compound (15 mg, 6%) as a light yellow solid. LC-MS (m/z)=335 [M+H]⁺. ¹H NMR (400 MHz, CDCl₃): δ 7.83 (s, 1H), 7.41-7.37 (m, 2H), 7.32-7.27 (m, 3H), 4.32 (dt, J=12.0, 4.4 Hz, 1H), 3.89-3.82 (m, 1H), 3.57 (quint, J=8.8 Hz, 1H), 3.23-3.16 (m, 2H), 2.97 (dd, J=17.6, 12.0 Hz, 1H), 2.43-2.38 (m, 1H), 2.15-2.04 (m, 3H), 2.00-1.80 (m, 4H), 1.75-1.65 (m, 2H).

### 2-Chloro-4-phenylpyridine

To a solution of 4-bromo-2-chloropyridine (2.0 g, 10.39 mmol) and tetrakis(triphenylphosphine)palladium (1.20 g, 1.04 mmol) in toluene (60 mL) was added phenylboronic acid (1.39 g, 11.43 mmol). A solution of sodium carbonate (1.32 g, 12.47 mmol) in water (12 mL) was added and the reaction was heated overnight at 90° C. under nitrogen atmosphere. Upon completion, the reaction mixture was cooled and partitioned between ethyl acetate (50 mL) and brine (40 ml). The aqueous layer was extracted with ethyl acetate (3×80 mL), and the combined organic phases were dried with sodium sulfate and concentrated to dryness. The residue was purified by column chromatography on silica gel (elution with petroleum ether: ethyl acetate=20/1-10/1) to give the title compound (1.58 g, 81%) as a light yellow solid. LC-MS (m/z)=190 [M+H]⁺.

### 2-(Benzyloxy)-4-phenylpyridine

To a solution of benzyl alcohol (4.53 g, 41.93 mmol) in DMF (35 mL) was added sodium hydride (60% in oil, 1.68 g, 41.9 mmol). The reaction mixture was stirred at room temperature under nitrogen for 30 min. 2-Chloro-4-phenylpyridine (1.58 g, 8.38 mmol) was added, and the reaction mixture was stirred overnight at 90° C. Upon completion, the reaction mixture was partitioned between ethyl acetate (50 mL) and brine (40 mL). The aqueous layer was extracted with ethyl acetate (3×80 mL) and the combined organic phases were dried with sodium sulfate and concentrated to dryness. The residue was purified by flash chromatography to give the title compound (1.42 g, 65%) as a light yellow solid. LC-MS (m/z)=262 [M+H]⁺.

### 4-Phenylpiperidin-2-one

To 2-(benzyloxy)-4-phenylpyridine (1.42 g, 5.44 mmol) suspended in ethanol (20 mL) under nitrogen was added palladium (10 wt. % on activated carbon) (710 mg). The reaction mixture was deoxygenated under vacuum, and hydrogenated at atmospheric pressure overnight. Upon completion, the reaction mixture was filtered through a pad of Celite and washed with MeOH (2×40 mL). The filtrate was concentrated to give the title compound (1.12 g, 100%) as a white solid, which was used without further purification. LC-MS (m/z)=176 [M+H]⁺.

### 1-Amino-2-cyclopentyl-1H-imidazole-5-carboxylic acid

To a mixture of methyl 1-amino-2-cyclopentyl-1H-imidazole-5-carboxylate (5.0 g, 19 mmol) in THF/water (20 mL/5 mL) was added sodium hydroxide (3.8 g, 96 mmol). The reaction was stirred at room temperature overnight. Upon completion, the organic solvent was removed in vacuo and the aqueous solution was neutralized with 3 N HCl. The precipitate was collected, washed with water and concentrated to dryness. The title compound (4.3 g, 92%) was obtained as a white solid. LC-MS (m/z)=196 [M+H]⁺.

The compounds of Examples 135 to 137 were synthesized in a similar manner to Example 134.

TABLE 14

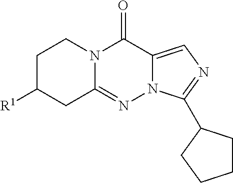

| No. | R¹ | ¹H NMR |
|---|---|---|
| 135 | 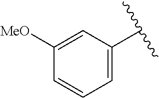 [SMILES: *c1cccc(OC)c1] | (400 MHz, CDCl₃): δ 7.81 (s, 1H), 7.32-7.27 (m, 1H), 6.86-6.81 (m, 3H), 4.32 (dt, J = 14.0, 4.8 Hz, 1H), 3.87- 3.79 (m, 1H), 3.83 (s, 3H), 3.56 (quint, J = 8.4 Hz, 1H), 3.20-3.12 (m, 2H), 2.99-2.91 (m, 1H), 2.41-2.36 (m, 1H), 2.11-2.00 (m, 3H), 2.00-1.80 (m, 4H), 1.75-1.67 (m, 2H). |
| 136 | 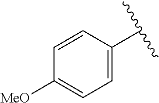 [SMILES: *c1ccc(OC)cc1] | (400 MHz, CDCl₃): δ 7.81 (s, 1H), 7.18 (d, J = 8.8 Hz, 2H), 6.91 (d, J = 8.8 Hz, 2H), 4.31 (dt, J = 13.6, 5.2 Hz, 1H), 3.83 (s, 3H), 3.86-3.78 (m, 1H), 3.57 (quint, J = 8.0 Hz, 1H), 3.18-3.12 (m, 2H), 2.95-2.88 (m, 1H), 2.39-2.34 (m, 1H), 2.15-1.82 (m, 7H), 1.75-1.67 (m, 2H). |
| 137 | H | (400 MHz, CDCl₃): δ 7.79 (s, 1H), 4.39 (t, J = 6.0 Hz, |
| | | 2H), 3.57 (quint, J = 8.0 Hz, 1H), 2.83 (t, J = 6.8 Hz, |
| | | 2H), 2.18-2.08 (m, 2H), 2.00-1.80 (m, 8H), 1.76-1.67 |
| | | (m, 2H). |

### Example 138

### 7-(4-Chlorophenyl)-3-(tetrahydro-2H-pyran-4-yl)-7,8-dihydro-6H,10H-imidazo[5,1-f]pyrrolo[2,1-c][1,2,4]triazin-10-one

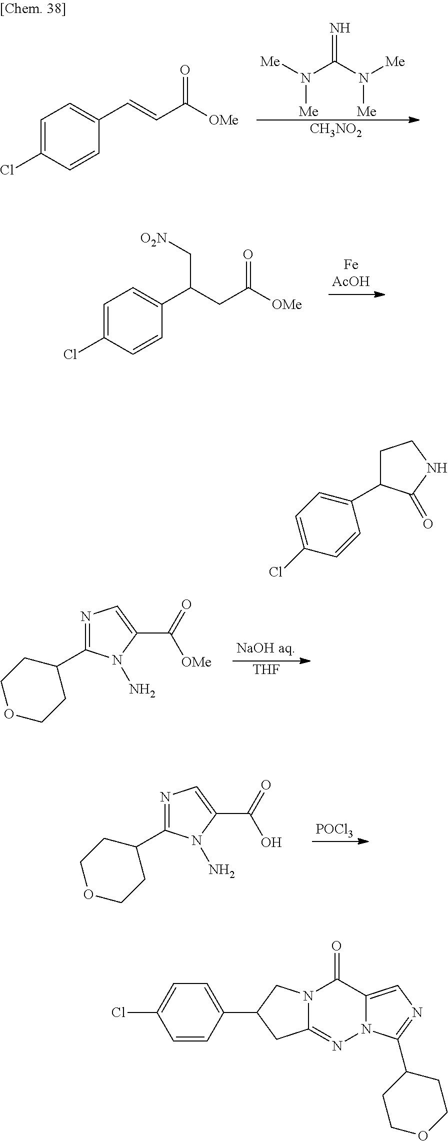

To a solution of 4-(4-chlorophenyl)pyrrolidin-2-one (0.06 g, 0.3 mmol) in toluene (0.3 mL) was added phosphoryl trichloride (0.03 g, 0.18 mmol) in toluene (0.3 mL) at 10° C. The reaction was stirred at ambient temperature for 3 h. A solution of 1-amino-2-(tetrahydro-2H-pyran-4-yl)-1H-imidazole-5-carboxylic acid (0.04 g, 0.2 mmol) in toluene (0.3 mL) was added. The reaction mixture was heated and stirred at that temperature for 16 h then cooled to room temperature, and the toluene was decanted. Water (10 mL) and DCM (30 mL) was added to the reaction. 5 M aqueous NaOH was added to the reaction to adjust the pH to 7. The layers were separated and the organic phase was washed with water and brine. The combined organics were dried over anhydrous Na₂SO₄, filtered and concentrated in vacuo. The resulting mixture was purified by reverse phase HPLC to provide the title compound (0.02 g, 0.06 mmol, 18%) as a white solid. LC-MS (m/z)=371 [M+H]⁺. ¹H NMR (CDCl₃, 400 MHz): δ 7.84 (s, 1H), 7.39 (d, J=8.4 Hz, 2H) 7.23 (d, J=8.0 Hz, 2H), 4.55 (dd, J=12.0, 8.0 Hz, 1H), 4.11 (d, J=11.6 Hz, 2H), 4.02-3.97 (m, 1H), 3.85-3.80 (m, 1H), 3.61 (t, J=11.0 Hz, 2H), 3.49-3.43 (m, 2H), 3.23-3.17 (m, 1H), 2.13-2.09 (m, 2H), 1.94 (d, J=12.8 Hz, 2H).

### Methyl 3-(4-chlorophenyl)-4-nitrobutanoate

The mixture of (E)-methyl 3-(4-chlorophenyl)acrylate (1.97 g, 10 mmol) and 1,1,3,3-tetramethylguanidine (0.21 g, 1.8 mmol) in methyl nitroperoxoite (6.16 g, 80 mmol) was stirred at ambient temperature for 2 days. The mixture was evaporated and then EtOAc (50 mL) was added. The mixture was washed with 1 M aqueous HCl (20 mL×2). The combined organics were dried over anhydrous Na₂SO₄, filtered and concentrated in vacuo. The resulting oil was purified by flash column chromatography with a gradient elution of EtOAc (5%) and hexanes (95%) to EtOAc (10%) and hexanes (90%) to provide the title compound (1.55 g, 6 mmol, 60%) as a colorless oil. LC-MS (m/z)=258 [M+H]⁺.

### 4-(4-Chlorophenyl)pyrrolidin-2-one

To a solution of methyl 3-(4-chlorophenyl)-4-nitrobutanoate (1.5 g, 5.82 mmol) in acetic acid (10 mL) was added iron (0.98 g, 17.46 mmol). The reaction mixture was stirred at room temperature for 16 h. HCl (20 mL) and ice (20 g) was added to the reaction vessel and the mixture was extracted with DCM. The organic phase was dried over anhydrous Na₂SO₄, filtered and concentrated in vacuum. The mixture was distilled with toluene to remove acetic acid whereupon crystallization took place. The crude precipitate was washed with diethyl ether and dried in vacuo to provide the title compound (0.91 g, 4.66 mmol, 87%) as a white solid. LC-MS (m/z)=196 [M+H]⁺.

### Amino-2-(tetrahydro-2H-pyran-4-yl)-1H-imidazole-5-carboxylic acid

A mixture of methyl 1-amino-2-(tetrahydro-2H-pyran-4-yl)-1H-imidazole-5-carboxylate (225 mg, 1 mmol) and NaOH aq. (2 N, 1.5 mL) in MeOH (2 mL) was stirred at room temperature for 5 h. 6 N HCl aq. was added dropwise to adjust the pH to 4-5. The mixture was filtrated, washed with water to give crude compound (200 mg, 0.95 mmol, 95%) as white solid. LC-MS (m/z)=211 [M+H]⁺.

The compounds of Reference Examples 20 to 23 were synthesized in a similar manner to Reference Example 1.

TABLE 15

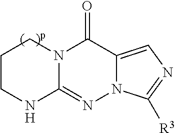

| No. | p | R³— | ¹H NMR |
|---|---|---|---|
| 20 | 0 | 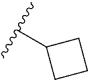 [SMILES: *C1CCC1] | (400 MHz, CDCl₃): δ 1.94-2.02 (m, 1H), 2.04-2.14 (m, 1H) 2.32-2.40 (m, 2H), 2.45-2.55 (m, 2H), 3.76- 3.82 (m, 2H), 3.84-3.90 |
| | | | (m, 1H), 4.17 (t, J = 8.0 Hz, |
| | | | 2H), 4.72 (s, 1H), 7.77 (s, 1H). |
| 21 | 0 | 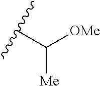 [SMILES: *C(C)OC] | (400 MHz, CDCl₃): δ 1.64 (d, J = 12 Hz, 3H), 3.28 (s, 3H), 3.78-3.82 (m, 2H), 4.17-4.21 (m, 2H), 4.84- 4.86 (m, 1H), 5.08 (s, 1H), 7.79 (s, 1H). |
| 22 | 1 | 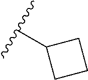 [SMILES: *C1CCC1] | (400 MHz, CDCl₃): δ 1.95-2.12 (m, 4H), 2.32-2.40 (m, 2H), 2.45-2.55 (m, 2H), 3.44-3.48 (m, 2H), 3.83- 3.92 (m, 1H), 4.01 (t, J = |
| | | | 6.0 Hz, 2H), 4.85 (br, 1H), |
| | | | 7.76 (s, 1H). |
| 23 | 1 | 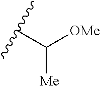 [SMILES: *C(C)OC] | (400 MHz, CDCl₃): δ 1.64 (d, J = 7.2 Hz, 3H), 2.08- 2.11 (m, 2H), 3.28 (s, 3H), 3.44-3.47 (m, 2H), 4.01- 4.04 (m, 2H), 4.84-4.87 (m, 1H), 4.97 (s, 1H), 7.79 |
| | | | (s, 1H). |

The compounds of Reference Examples 24 to 28 were synthesized in a similar manner to Reference Example 6.

TABLE 16

| No. | Structure | ¹H NMR or LC-MS |
|---|---|---|
| 24 | 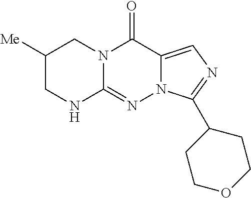 [SMILES: CC1CNc2nn3c(C4CCOCC4)ncc3c(=O)n2C1] | (400 MHz, CDCl₃): 1.15 (d, J = 6.8 Hz, 3H), 1.80-1.86 (m, 4H), 1.90-2.09 (m, 2H), 3.52- 3.59 (m, 4H), 4.06-4.10 (m, 4H), 4.92 (s, 1H), 7.75 (s, 1H). |
| 25 | 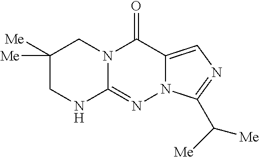 [SMILES: CC(C)c1ncc2c(=O)n3c(nn12)NCC(C)(C)C3] | LC-MS (m/z) = 262 [M + H]⁺ |
| 26 | 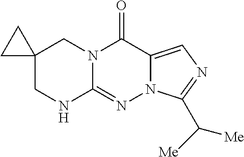 [SMILES: CC(C)c1ncc2c(=O)n3c(nn12)NCC1(CC1)C3] | LC-MS (m/z) = 259 [M + H]⁺ |
| 27 | 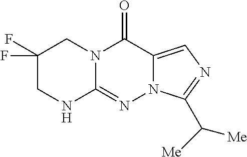 [SMILES: CC(C)c1ncc2c(=O)n3c(nn12)NCC(F)(F)C3] | LC-MS (m/z) = 270 [M + H]⁺ |
| 28 | 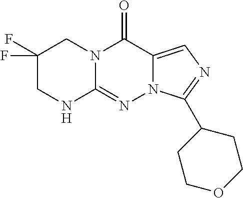 [SMILES: O=c1c2cnc(C3CCOCC3)n2nc2n1CC(F)(F)CN2] | LC-MS (m/z) = 312 [M + H]⁺ |

The compounds of Reference Examples 29 and 30 were synthesized in a similar manner to Reference Example 16.

TABLE 17

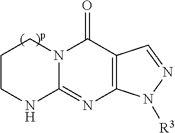

| No. | p | R³— | ¹H NMR or LC-MS |
|---|---|---|---|
| 29 | 0 | 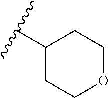 [SMILES: *C1CCOCC1] | LC-MS (m/z) = 262 [M + H]⁺ |
| 30 | 1 | 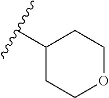 [SMILES: *C1CCOCC1] | LC-MS (m/z) = 276 [M + H]⁺ |

The compounds of Examples 139 to 229 were synthesized in a similar manner to Example 1, 2 or 3.

TABLE 18-1

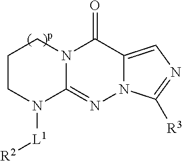

| No. | p | R²—L¹— | R³— | ¹H NMR |
|---|---|---|---|---|
| 139 | 0 | 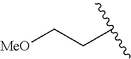 [SMILES: *CCOC] | 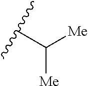 [SMILES: *C(C)C] | (400 MHz, CDCl₃): δ 1.36 (d, J = 7.2 Hz, 6H), 3.38-3.41 (m, 4H), 3.54-3.56 (m, 2H), 3.65-3.67 (m, 2H), 3.77-3.80 (m, 2H), 4.05-4.09 (m, 2H), 7.71 (s, 1H). |
| 140 | 0 | 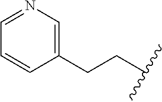 [SMILES: *CCc1cccnc1] | 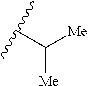 [SMILES: *C(C)C] | (400 MHz, CDCl₃): δ 1.37 (s, 3H), 1.39 (s, 3H), 3.02 (t, J = 7.2 Hz, 2H), 3.36-3.47 (m, 1H), 3.59- 3.65 (m, 4H), 4.06 (t, J = 8.0 Hz, 2H), 7.27-7.29 (m, 1H), 7.58-7.61 (m, 1H), 7.73 (s, 1H), 8.51-8.55 (m, 2H). |
| 141 | 0 | 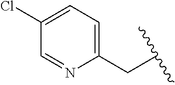 [SMILES: *Cc1ccc(Cl)cn1] | 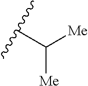 [SMILES: *C(C)C] | (400 MHz, DMSO-d₆): δ 1.19 (d, J = 7.2 Hz, 6H), 3.20-3.32 (m, 1H), 3.72-3.76 (m, 2H), 4.00-4.04 (m, 2H), 4.61 (s, 2H), 7.53-7.55 (m, 2H), 7.93-7.98 (m, 1H), 8.61- 8.62 (m, 1H). |
| 142 | 0 | 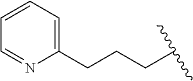 [SMILES: *CCCc1ccccn1] | 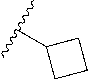 [SMILES: *C1CCC1] | (400 MHz, CDCl₃): δ 1.98-2.20 (m, 4H), 2.34-2.41 (m, 2H), 2.48- 2.56 (m, 2H), 2.89-2.93 (t, J = 7.4 Hz, 2H), 3.43-3.46 (t, J = 7.0 Hz, 2H), 3.62-3.66 (t, J = 8.0 Hz, 2H), 3.88-3.93 (m, 1H), 3.98-4.02 (t, J = |
| | | | | 8.0 Hz, 2H), 7.13-7.17 (m, 2H), |
| | | | | 7.55-7.59 (m, 1H), 7.74 (s, 1H), |
| | | | | 8.55-8.56 (d, J = 4.8 Hz, 1H). |
| 143 | 0 | 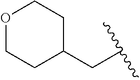 [SMILES: *CC1CCOCC1] | 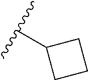 [SMILES: *C1CCC1] | (400 MHz, CDCl₃): δ 1.38-1.49 (m, 2H), 1.63-1.69 (m, 2H), 1.96- 2.04 (m, 2H), 2.06-2.15 (m, 1H), 2.34-2.42 (m, 2H), 2.49-2.58 (m, 2H), 3.25 (d, J = 7.2 Hz, 2H), 3.39-3.45 (m, 2H), 3.69 (t, J = 8.0 |
| | | | | Hz, 2H), 3.88-3.94 (m, 1H), 4.01- |
| | | | | 4.05 (m, 2H), 4.08-4.12 (m, 2H), |
| | | | | 7.75 (s, 1H). |
| 144 | 0 | 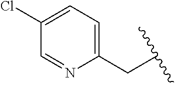 [SMILES: *Cc1ccc(Cl)cn1] | 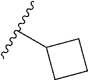 [SMILES: *C1CCC1] | (400 MHz, CDCl₃): δ 1.96-2.12 (m, 2H), 2.31-2.39 (m, 2H), 2.46- 2.54 (m, 2H), 3.75-3.79 (m, 2H), 3.88-3.93 (m, 1H), 4.09-4.13 (m, 2H), 4.63 (s, 2H), 7.36-7.38 (m, 1H), 7.69-7.72 (m, 1H), 7.76 (s, |
| | | | | 1H), 8.55 (d, J = 2.4 Hz, 1H). |
| 145 | 0 | 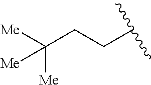 [SMILES: *CCC(C)(C)C] | 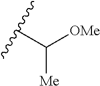 [SMILES: *C(C)OC] | (400 MHz, CDCl₃): δ 1.00 (s, 9H), 1.54-1.58 (t, J = 8.4 Hz, 2H), 1.64-1.66 (m, 3H), 3.34-3.42 (m, 5H), 3.66-3.70 (m, 2H), 4.07-4.11 (m, 2H), 4.87-4.92 (m, 1H), 7.79 (s, 1H). |
| 146 | 0 | 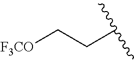 [SMILES: *CCOC(F)(F)F] | 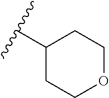 [SMILES: *C1CCOCC1] | (400 MHz, CDCl₃): δ 1.87-1.91 (m, 2H), 2.02-2.12 (m, 2H), 3.28- 3.35 (m, 1H), 3.54-3.60 (m, 2H), 3.70 (t, J = 5.0 Hz, 2H), 3.82 (t, J = 8.0 Hz, 2H), 4.07-4.15 (m, 4H), 4.27 (t, J = 5.0 Hz, 2H), 7.74 (s, 1H). |
| 147 | 0 | 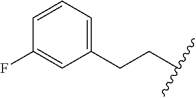 [SMILES: *CCc1cccc(F)c1] | 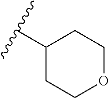 [SMILES: *C1CCOCC1] | (400 MHz, CDCl₃): δ 1.91-1.94 (m, 2H), 2.04-2.14 (m, 2H), 2.99 (t, J = 7.2 Hz, 2H), 3.30-3.38 (m, 1H), 3.57-3.64 (m, 6H), 4.03-4.12 (m, 4H), 6.94-7.03 (m, 3H), 7.27- 7.32 (m, 1H), 7.73 (s, 1H). |
| 148 | 0 | 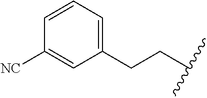 [SMILES: *CCc1cccc(C#N)c1] | 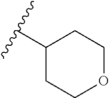 [SMILES: *C1CCOCC1] | (400 MHz, CDCl₃): δ 1.89-1.93 (m, 2H), 2.05-2.16 (m, 2H), 3.05 (t, J = 7.2 Hz, 2H), 3.30-3.38 (m, 1H), 3.58-3.65 (m, 6H), 4.05-4.12 (m, 4H), 7.46 (t, J = 8.0 Hz, 1H), 7.51 (d, J = 8.0 Hz, 1H), 7.56- 7.57 (m, 2H), 7.73 (s, 1H). |
| 149 | 0 | 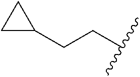 [SMILES: *CCC1CC1] | 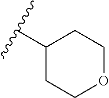 [SMILES: *C1CCOCC1] | (400 MHz, CDCl₃): δ 0.11-0.15 (m, 2H), 0.50-0.54 (m, 2H), 0.70- 0.76 (m, 1H), 1.54-1.60 (m, 2H), 1.91-1.94 (m, 2H), 2.02-2.14 (m, 2H), 3.29-3.37 (m, 1H), 3.42-3.46 (m, 2H), 3.56-3.62 (m, 2H), 3.68- 3.72 (m, 2H), 4.07-4.11 (m, 4H), |
| | | | | 7.72 (s, 1H). |
| 150 | 0 | 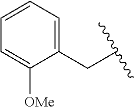 [SMILES: *Cc1ccccc1OC] | 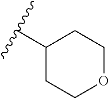 [SMILES: *C1CCOCC1] | (400 MHz, CDCl₃): δ 1.92-1.96 (m, 2H), 2.06-2.16 (m, 2H), 3.35- 3.43 (m, 1H), 3.58-3.69 (m, 4H), 3.88 (s, 3H), 4.04-4.13 (m, 4H), 4.55 (s, 2H), 6.93-6.97 (m, 2H), 7.31-7.38 (m, 2H), 7.73 (s, 1H). |
| 151 | 0 | 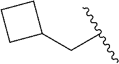 [SMILES: *CC1CCC1] | 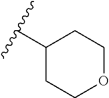 [SMILES: *C1CCOCC1] | (400 MHz, CDCl₃): δ 1.94-2.19 (m, 10H), 2.64-2.73 (m, 1H), 3.32-3.39 (m, 3H), 3.58-3.66 (m, 4H), 4.06-4.11 (m, 4H), 7.73 (s, 1H). |
| 152 | 0 | 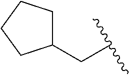 [SMILES: *CC1CCCC1] | 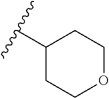 [SMILES: *C1CCOCC1] | (400 MHz, CDCl₃): δ 1.30-1.37 (m, 2H), 1.60-1.84 (m, 6H), 1.90- 1.94 (m, 2H), 2.02-2.12 (m, 2H), 2.24-2.32 (m, 1H), 3.26-3.37 (m, 3H), 3.56-3.62 (m, 2H), 3.68-3.72 (m, 2H), 4.08-4.12 (m, 4H), 7.73 (s, 1H). |
| 153 | 0 | 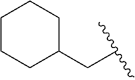 [SMILES: *CC1CCCCC1] | 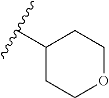 [SMILES: *C1CCOCC1] | (400 MHz, CDCl₃): δ 0.99-1.08 (m, 2H), 1.23-1.31 (m, 3H), 1.69- 1.77 (m, 6H), 1.90-1.94 (m, 2H), 2.02-2.12 (m, 2H), 3.17-3.19 (m, 2H), 3.27-3.37 (m, 1H), 3.56-3.69 (m, 4H), 4.07-4.11 (m, 4H), 7.72 (s, 1H). |
| 154 | 0 | 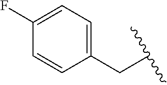 [SMILES: *Cc1ccc(F)cc1] | 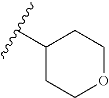 [SMILES: *C1CCOCC1] | (400 MHz, CDCl₃): δ 1.90-1.94 (m, 2H), 2.07-2.12 (m, 2H), 3.32- 3.40 (m, 1H), 3.54-3.61 (m, 4H), 4.06-4.11 (m, 4H), 4.51 (s, 2H), 7.06-7.10 (m, 2H), 7.32-7.35 (m, 2H), 7.75 (s, 1H). |
| 155 | 0 | 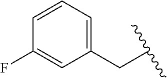 [SMILES: *Cc1cccc(F)c1] | 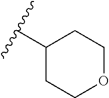 [SMILES: *C1CCOCC1] | (400 MHz, CDCl₃): δ 1.90-1.94 (m, 2H), 2.04-2.14 (m, 2H), 3.32- 3.41 (m, 1H), 3.55-3.62 (m, 4H), 4.08-4.12 (m, 4H), 4.53 (s, 2H), 7.03-7.14 (m, 3H), 7.34-7.39 (m, 1H), 7.76 (s, 1H). |
| 156 | 0 | 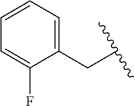 [SMILES: *Cc1ccccc1F] | 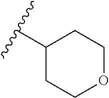 [SMILES: *C1CCOCC1] | (400 MHz, CDCl₃): δ 1.91-1.95 (m, 2H), 2.06-2.12 (m, 2H), 3.32- 3.41 (m, 1H), 3.58-3.70 (m, 4H), 4.06-4.13 (m, 4H), 4.59 (s, 2H), 7.10-7.18 (m, 2H), 7.34-7.45 (m, 2H), 7.74 (s, 1H). |
| 157 | 0 | 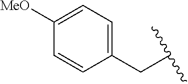 [SMILES: *Cc1ccc(OC)cc1] | 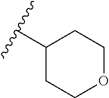 [SMILES: *C1CCOCC1] | (400 MHz, CDCl₃): δ 1.92-1.96 (m, 2H), 2.08-2.12 (m, 2H), 3.36- 3.40 (m, 1H), 3.53-3.63 (m, 4H), 3.82 (s, 3H), 4.03-4.12 (m, 4H), 4.48 (s, 2H), 6.90-6.92 (d, J = 8.4 Hz, 2H), 7.27-7.29 (d, J = 7.2 Hz, 2H), 7.74 (s, 1H). |
| 158 | 0 | 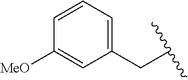 [SMILES: *Cc1cccc(OC)c1] | 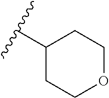 [SMILES: *C1CCOCC1] | (400 MHz, CDCl₃): δ 1.91-1.95 (m, 2H), 2.04-2.16 (m, 2H), 3.32- 3.43 (m, 1H), 3.56-3.62 (m, 4H), 3.82 (s, 3H), 4.06-4.10 (m, 4H), 4.51 (s, 2H), 6.87-6.94 (m, 3H), 7.29-7.31 (m, 1H), 7.75 (s, 1H). |
| 159 | 0 | 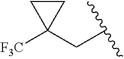 [SMILES: *CC1(C(F)(F)F)CC1] | 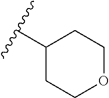 [SMILES: *C1CCOCC1] | (400 MHz, CDCl₃): δ 0.90 (s, 2H), 1.17 (t, J = 6.0 Hz, 2H), 1.88-1.92 (m, 2H), 2.03-2.13 (m, 2H), 3.26-3.34 (m, 1H), 3.54-3.61 (m, 4H), 3.79 (d, J = 8.0 Hz, 2H), 4.07-4.11 (m, 4H), 7.74 (s, 1H). |
| 160 | 0 | 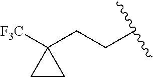 [SMILES: *CCC1(C(F)(F)F)CC1] | 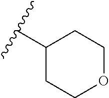 [SMILES: *C1CCOCC1] | (400 MHz, CDCl₃): δ 0.70 (s, 2H), 1.09 (t, J = 6.0 Hz, 2H), 1.90-1.95 (m, 4H), 2.01-2.11 (m, 2H), 3.27-3.33 (m, 1H), 3.45-3.60 (m, 4H), 3.70 (t, J = 7.8 Hz, 2H), 4.07-4.11 (m, 4H), 7.73 (s, 1H). |
| 161 | 0 | 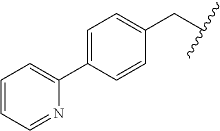 [SMILES: *Cc1ccc(-c2ccccn2)cc1] | 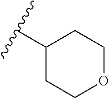 [SMILES: *C1CCOCC1] | (400 MHz, CDCl₃): δ 1.89-1.93 (m, 2H), 2.10 (ddd, J = 24.7, 12.1, 4.0 Hz, 2H), 3.36-3.41 (m, 1H), 3.54-3.61 (m, 4H), 4.06-4.10 (m, 4H). 4.58 (s, 2H), 7.24-7.28 (m, 1H), 7.44 (d, J = 8.3 Hz, 2H), 7.71-7.79 (m, 3H), 8.00 (d, J = 8.3 Hz, 2H), 8.69 (d, J = 4.6 Hz, 1H). |
| 162 | 0 | 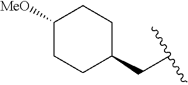 [SMILES: *C[C@H]1CC[C@H](OC)CC1] | 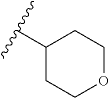 [SMILES: *C1CCOCC1] | (400 MHz, CDCl₃): δ 1.04-1.14 (m, 2H), 1.17-1.27 (m, 2H), 1.67- 1.77 (m, 1H), 1.83-1.93 (m, 4H), 2.03-2.15 (m, 4H), 3.12-3.18 (m, 1H), 3.19 (d, J = 7.1 Hz, 2H), 3.28-3.35 (m, 1H), 3.37 (s, 3H), 3.59 (td, J = 11.6, 2.3 Hz, 2H), |
| | | | | 3.67 (t, J = 8.0 Hz, 2H), 4.07-4.12 |
| | | | | (m, 4H), 7.73 (s, 1H). |
| 163 | 0 | 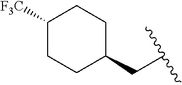 [SMILES: *C[C@H]1CC[C@H](C(F)(F)F)CC1] | 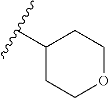 [SMILES: *C1CCOCC1] | (400 MHz, CDCl₃): δ 1.04-1.15 (m, 2H), 1.30-1.40 (m, 2H), 1.69- 1.78 (m, 1H), 1.88-1.95 (m, 4H), 2.01-2.13 (m, 5H), 3.21 (d, J = 7.1 Hz, 2H), 3.27-3.35 (m, 1H), 3.59 (td, J = 11.6, 2.1 Hz, 2H), 3.68 (t, J = 7.9 Hz, 2H), 4.07-4.13 (m, |
| | | | | 4H), 7.73 (s, 1H). |
| 164 | 0 | 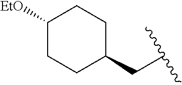 [SMILES: *C[C@H]1CC[C@H](OCC)CC1] | 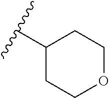 [SMILES: *C1CCOCC1] | (400 MHz, CDCl₃): δ 1.07-1.14 (m, 2H), 1.19-1.30 (m, 2H), 1.21 (t, J = 7.1 Hz, 3H), 1.66-1.75 (m, 1H), 1.82-1.93 (m, 4H), 2.03-2.13 (m, 4H), 3.19 (d, J = 7.3 Hz, 2H), 3.21-3.35 (m, 2H), 3.54 (q, J = 7.1 Hz, 2H), 3.59 (td, J = 11.6, 2.4 |
| | | | | Hz, 2H), 3.67 (t, J = 8.0 Hz, 2H), |
| | | | | 4.07-4.12 (m, 4H), 7.73 (s, 1H). |
| 165 | 0 | 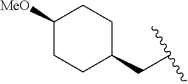 [SMILES: *C[C@H]1CC[C@@H](OC)CC1] | 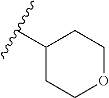 [SMILES: *C1CCOCC1] | (400 MHz, CDCl₃): δ 1.38-1.56 (m, 6H), 1.79 (brs, 1H), 1.89-1.97 (m, 4H), 2.01-2.11 (m, 2H), 3.23 (d, J = 7.1 Hz, 2H), 3.30-3.37 (m, 1H), 3.33 (s, 3H), 3.44-3.47 (m, 1H), 3.61 (td, J = 11.7, 2.2 Hz, 2H), 3.67 (t, J = 8.0 Hz, 2H), |
| | | | | 4.06-4.11 (m, 4H), 7.73 (s, 1H). |
| 166 | 0 | 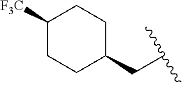 [SMILES: *C[C@H]1CC[C@@H](C(F)(F)F)CC1] | 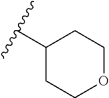 [SMILES: *C1CCOCC1] | (400 MHz, CDCl₃): δ 1.57-1.76 (m, 8H), 1.86-1.91 (m, 2H), 1.99- 2.14 (m, 4H), 3.27-3.35 (m, 1H), 3.42 (d, J = 8.3 Hz, 2H), 3.55 (td, J = 11.8, 2.1 Hz, 2H), 3.68 (t, J = 8.0 Hz, 2H), 4.05-4.14 (m, 4H), 7.74 (s, 1H). |
| 167 | 0 | 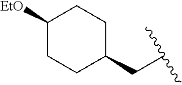 [SMILES: *C[C@H]1CC[C@@H](OCC)CC1] | 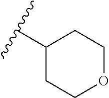 [SMILES: *C1CCOCC1] | (400 MHz, CDCl₃): δ 1.21 (t, J = 7.0 Hz, 3H), 1.45-1.54 (m, 6H), 1.78-1.94 (m, 5H), 2.01-2.11 (m, 2H), 3.24 (d, J = 7.3 Hz, 2H), 3.34 (tt, J = 11.5, 3.8 Hz, 1H), 3.47 (q, J = 7.0 Hz, 2H), 3.53 (br s, 1H), 3.60 (td, J = 11.6, 2.3 Hz, |
| | | | | 2H), 3.68 (t, J = 8.0 Hz, 2H), |
| | | | | 4.06-4.11 (m, 4H), 7.72 (s, 1H). |
| 168 | 0 | 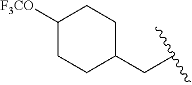 [SMILES: *CC1CCC(OC(F)(F)F)CC1] | 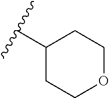 [SMILES: *C1CCOCC1] | (400 MHz, CDCl₃): δ 1.10-1.21 (m, 1H), 1.45-1.56 (m, 2H), 1.60- 1.66 (m, 2H), 1.67-1.88 (m, 1H), 1.84-1.91 (m, 3H), 2.00-2.08 (m, 3H), 2.13-2.17 (m, 1H), 3.19-3.21 (d, J = 7.2 Hz, 1H), 3.24-3.26 (d, J = 7.2 Hz, 1H), 3.29-3.36 (m, |
| | | | | 1H), 3.61-3.65 (m, 2H), 3.65-3.70 |
| | | | | (m, 2H), 4.05-4.13 (m, 4H), 4.20- |
| | | | | 4.51 (m, 1H), 7.70 (s, 1H). |
| 169 | 0 | 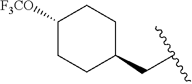 [SMILES: *C[C@H]1CC[C@H](OC(F)(F)F)CC1] | 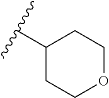 [SMILES: *C1CCOCC1] | (400 MHz, CDCl₃): δ 1.12-1.22 (m, 2H), 1.51-1.58 (m, 2H), 1.70- 1.78 (m, 1H), 1.89-1.92 (m, 4H), 2.10-2.14 (m, 2H), 2.16-2.19 (m, 2H), 3.20-3.22 (d, J = 7.2 Hz, 2H), 3.28-3.35 (m, 1H), 3.56-3.59 (m, 2H), 3.68-3.70 (m, 2H), 4.07- |
| | | | | 4.15 (m, 4H), 4.15-4.22 (m, 1H), |
| | | | | 7.73 (s, 1H). |
| 170 | 0 | 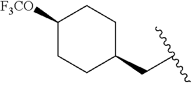 [SMILES: *C[C@H]1CC[C@@H](OC(F)(F)F)CC1] | 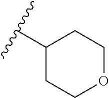 [SMILES: *C1CCOCC1] | (400 MHz, CDCl₃): δ 1.47-1.55 (m, 3H), 1.56-1.68 (m, 3H), 1.82- 1.87 (m, 1H), 1.90-1.94 (m, 2H), 2.02-2.12 (m, 4H), 2.25-2.27 (d, J = 7.2 Hz, 2H), 3.30-3.37 (m, 1H), 3.57-3.60 (m, 2H), 3.69-3.71 (m, 2H), 4.07-4.13 (m, 4H), 4.53 (s, |
| | | | | 1H), 7.73 (s, 1H). |
| 171 | 0 | 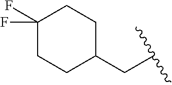 [SMILES: *CC1CCC(F)(F)CC1] | 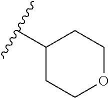 [SMILES: *C1CCOCC1] | (300 MHz, CDCl₃): δ 1.44 (d, J = 12.5 Hz, 2H), 1.70-1.91 (m, 6H), 2.10 (tt, J = 18.3, 6.4 Hz, 5H), 3.31 (dt, J = 16.6, 4.8 Hz, 3H), 3.59 (td, J = 11.4, 2.2 Hz, 2H), 3.70 (t, J = 7.7 Hz, 2H), 4.11 (q, J = 7.6 Hz, 4H), 7.76 (s, 1H). |
| 172 | 0 | 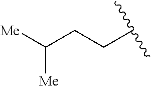 [SMILES: *CCC(C)C] | 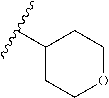 [SMILES: *C1CCOCC1] | (300 MHz, CDCl₃): δ 0.99 (d, J = 6.6 Hz, 6H), 1.54 (dd, J = 14.3, 7.0 Hz, 2H), 1.71 (dd, J = 20.9, 14.3 Hz, 1H), 1.93 (d, J = 13.2 Hz, 2H), 2.06 (td, J = 12.1, 3.9 Hz, 2H), 3.35 (dt, J = 19.1, 6.6 Hz, 3H), 3.57 (td, J = 11.6, 2.4 |
| | | | | Hz, 2H), 3.66 (t, J = 8.1 Hz, 2H), |
| | | | | 4.09 (t, J = 8.1 Hz, 4H), 7.73 (s, |
| | | | | 1H). |
| 173 | 0 | 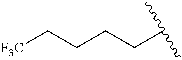 [SMILES: *CCCCC(F)(F)F] | 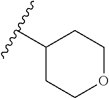 [SMILES: *C1CCOCC1] | (300 MHz, CDCl₃): δ 1.78 (ddt, J = 34.8, 21.6, 7.7 Hz, 6H), 2.16 (tdd, J = 23.6, 13.6, 4.8 Hz, 4H), 3.34-3.43 (m, 3H), 3.52-3.60 (m, 2H), 3.70 (t, J = 8.1 Hz, 2H), 4.11 (td, J = 11.2, 4.6 Hz, 4H), 7.81 (s, 1H). |
| 174 | 0 | 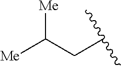 [SMILES: *CC(C)C] | 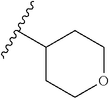 [SMILES: *C1CCOCC1] | (300 MHz, CDCl₃): δ 1.00 (d, J = 6.6 Hz, 6H), 1.89-2.15 (m, 5H), 3.15 (d, J = 7.3 Hz, 2H), 3.34 (td, J = 8.4, 5.4 Hz, 1H), 3.62 (td, J = 18.7, 6.5 Hz, 4H), 4.10 (t, J = 8.1 Hz, 4H), 7.74 (s, 1H). |
| 175 | 0 | 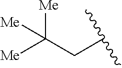 [SMILES: *CC(C)(C)C] | 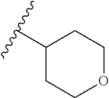 [SMILES: *C1CCOCC1] | (300 MHz, CDCl₃): δ 1.04 (d, J = 7.3 Hz, 9H), 1.91 (d, J = 11.0 Hz, 2H), 2.05 (td, J = 12.5, 4.4 Hz, 2H), 3.11 (s, 2H), 3.27-3.34 (m, 1H), 3.57 (td, J = 11.4, 2.4 Hz, 2H), 3.75 (t, J = 8.1 Hz, 2H), 4.10 (t, J = 8.1 Hz, 4H), 7.73 (s, 1H). |
| 176 | 0 | 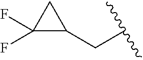 [SMILES: *CC1CC1(F)F] | 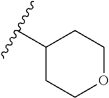 [SMILES: *C1CCOCC1] | (300 MHz, CDCl₃): δ 1.22 (dd, J = 13.2, 3.7 Hz, 1H), 1.58-1.67 (m, 1H), 1.90 (t, J = 6.6 Hz, 3H), 2.07 (dt, J = 18.1, 6.2 Hz, 2H), 3.08 (dd, J = 14.7, 9.5 Hz, 1H), 3.30- 3.38 (m, 1H), 3.62 (tt, J = 18.7, 6.7 Hz, 3H), 3.86 (tt, J = 17.6, 5.9 |
| | | | | Hz, 2H), 4.11 (q, J = 8.1 Hz, 4H), |
| | | | | 7.75 (s, 1H). |
| 177 | 1 | 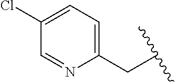 [SMILES: *Cc1ccc(Cl)cn1] | 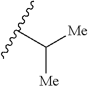 [SMILES: *C(C)C] | (400 MHz, DMSO-d₆): δ 1.07 (d, J = 8.0 Hz, 6H), 2.06-2.10 (m, 2H), 3.06-3.12 (m, 1H), 3.60-3.61 (m, 2H), 3.92-3.95 (m, 2H), 4.75 (s, 2H), 7.46-7.49 (m, 2H), 7.86- 7.88 (m, 1H), 8.57-8.58 (m, 1H). |
| 178 | 1 | 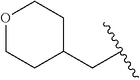 [SMILES: *CC1CCOCC1] | 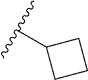 [SMILES: *C1CCC1] | (400 MHz, CDCl₃): δ 1.38-1.48 (m, 2H), 1.60-1.67 (m, 2H), 1.95- 2.03 (m, 1H), 2.05-2.25 (m, 4H), 2.33-2.41 (m, 2H), 2.52-2.62 (m, 2H), 3.36-3.47 (m, 6H), 3.84-3.91 (m, 1H), 4.00-4.04 (m, 4H), 7.74 (s, 1H). |
| 179 | 1 | 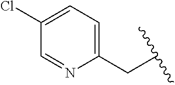 [SMILES: *Cc1ccc(Cl)cn1] | 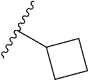 [SMILES: *C1CCC1] | (400 MHz, CDCl₃): δ 1.91-2.15 (m, 4H), 2.23-2.31 (m, 2H), 2.41- 2,51 (m, 2H), 3.61 (t, J = 6.0 Hz, 2H), 3.78-3.83 (m, 1H), 4.06 (t, J = 5.8 Hz, 2H), 4.79 (s, 2H), 7.37- 7.39 (m, 1H), 7.65-7.68 (m, 1H), |
| | | | | (s, 1H), 8.53 (d, J = 2.0 Hz, |
| | | | | 1H). |
| 180 | 1 | 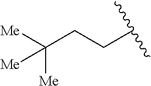 [SMILES: *CCC(C)(C)C] | 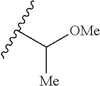 [SMILES: *C(C)OC] | (400 MHz, CDCl₃): δ 1.01 (s, 9H), 1.56-1.65 (m, 5H), 2.06- 2.09 (m, 2H), 3.33 (s, 3H), 3.41- 3.44 (m, 2H), 3.51-3.58 (m, 2H), 4.00-4.03 (m, 2H), 4.89-4.93 (m, 1H), 7.78 (s, 1H). |
| 181 | 1 | 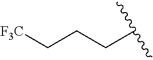 [SMILES: *CCCC(F)(F)F] | 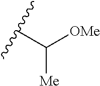 [SMILES: *C(C)OC] | (400 MHz, CDCl₃): δ 1.65 (d, J = 7.2 Hz, 3H), 2.00-2.21 (m, 6H), 3.30 (s, 3H), 3.44-3.47 (m, 2H), 3.59-3.62 (m, 2H), 4.02-4.05 (m, 2H), 4.85-4.88 (m, 1H), 7.79 (s, 1H). |
| 182 | 1 | 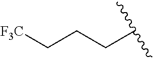 [SMILES: *CCCC(F)(F)F] | 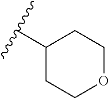 [SMILES: *C1CCOCC1] | (400 MHz, CDCl₃): δ 1.88-1.92 (m, 2H), 1.99-2.25 (m, 8H), 3.28- 3.34 (m, 1H), 3.45 (t, J = 6.0 Hz, 2H), 3.53-3.60 (m, 4H), 4.03 (t, J = 6.0 Hz, 2H), 4.07-4.12 (m, 2H), 7.74 (s, 1H). |
| 183 | 1 | 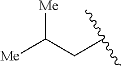 [SMILES: *CC(C)C] | 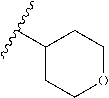 [SMILES: *C1CCOCC1] | (400 MHz, DMSO-d₆): δ 0.91 (d, J = 6.4 Hz, 6H), 1.81-1.87 (m, 4H), 1.96-1.99 (m, 2H), 2.15-2.19 (m, 1H), 3.23-3.32 (m, 3H), 3.39- 3.47 (m, 4H), 3.84-3.87 (m, 2H), 3.91-3.95 (m, 2H), 7.51 (s, 1H). |
| 184 | 1 | 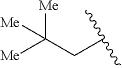 [SMILES: *CC(C)(C)C] | 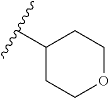 [SMILES: *C1CCOCC1] | (400 MHz, DMSO-d₆): δ 0.98 (s, 9H), 1.80-1.86 (m, 3H), 1.95-2.01 (m, 2H), 3.22-3.28 (m, 2H), 3.41- 3.48 (m, 6H), 3.87-3.90 (m, 2H), 3.92-3.97 (m, 2H), 7.53 (s, 1H). |
| 185 | 1 | 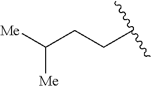 [SMILES: *CCC(C)C] | 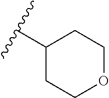 [SMILES: *C1CCOCC1] | (400 MHz, DMSO-d₆): δ 0.93 (d, J = 6.4 Hz, 6H), 1.52-1.64 (m, 3H), 1.82-1.87 (m, 4H), 1.94-2.00 (m, 2H), 3.04-3.30 (m, 1H), 3.40-3.48 (m, 6H), 3.84-3.87 (m, 2H), 3.93- 3.96 (m, 2H), 7.52 (s, 1H). |
| 186 | 1 | 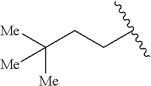 [SMILES: *CCC(C)(C)C] | 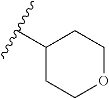 [SMILES: *C1CCOCC1] | (400 MHz, DMSO-d₆): δ 0.96 (s, 9H), 1.53-1.57 (m, 2H), 1.81-1.86 (m, 4H), 1.94-2.00 (m, 2H), 3.23- 3.29 (m, 2H), 3.36-3.39 (m, 3H), 3.42-3.49 (m, 2H), 3.84-3.87 (m, 2H), 3.92-3.96 (m, 2H), 7.53 (s, 1H). |
| 187 | 1 | 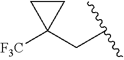 [SMILES: *CC1(C(F)(F)F)CC1] | 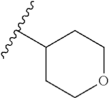 [SMILES: *C1CCOCC1] | (400 MHz, CDCl₃): δ 0.93 (s, 2H), 1.11-1.14 (m, 2H), 1.88-1.92 (m, 2H), 2.02-2.12 (m, 4H), 3.25- 3.33 (m, 1H), 3.50 (t, J = 6.0 Hz, 2H), 3.53-3.60 (m, 2H), 3.88 (s, 2H), 4.02 (t, J = 6.0 Hz, 2H), 4.08-4.12 (m, 2H), 7.74 (s, 1H). |
| 188 | 1 | 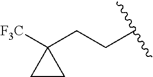 [SMILES: *CCC1(C(F)(F)F)CC1] | 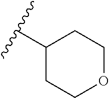 [SMILES: *C1CCOCC1] | (400 MHz, CDCl₃): δ 0.69 (s, 2H), 1.08 (t, J = 6.0 Hz, 2H), 1.88-1.97 (m, 4H), 2.02-2.12 (m, 4H), 3.27-3.34 (m, 1H), 3.43 (t, J = 6.0 Hz, 2H), 3.54-3.60 (m, 2H), 3.63-3.67 (m, 2H), 4.00 (t, J = 6.0 Hz, 2H), 4.07-4.11 (m, 2H), 7.73 |
| | | | | (s, 1H). |
| 189 | 1 | 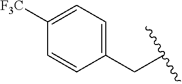 [SMILES: *Cc1ccc(C(F)(F)F)cc1] | 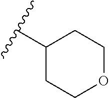 [SMILES: *C1CCOCC1] | (400 MHz, CDCl₃): δ 1.69-1.73 (m, 2H), 1.91-2.01 (m, 2H), 2.11- 2.17 (m, 2H), 3.16 (tt, J = 11.7, 4,0 Hz, 1H), 3.41-3.51 (m, 4H), 3.99 (dq, J = 11.7, 2.2 Hz, 2H), 4.06-4.09 (m, 2H), 4.78 (s, 2H), 7.44 (d, J = 8.1 Hz, 2H), 7.62 (d, |
| | | | | J = 8.1 Hz, 2H), 7.73 (s, 1H). |
| 190 | 1 | 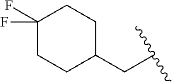 [SMILES: *CC1CCC(F)(F)CC1] | 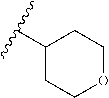 [SMILES: *C1CCOCC1] | (400 MHz, CDCl₃): δ 1.35-1.46 (m, 2H), 1.63-1.91 (m, 5H), 2.06- 2.18 (m, 8H), 3.23-3.32 (m, 1H), 3.40 (t, J = 7.0 Hz, 2H), 3.44-3.48 (m, 2H), 3.54 (td, J = 12.0, 1.8 Hz, 2H), 3.98-4.03 (m, 2H), 4.05- 4.11 (m, 2H), 7.72 (s, 1H). |
| 191 | 1 | 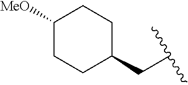 [SMILES: *C[C@H]1CC[C@H](OC)CC1] | 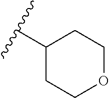 [SMILES: *C1CCOCC1] | (400 MHz, CDCl₃): δ 1.01-1.22 (m, 4H), 1.83-1.96 (m, 5H), 2.07- 2.17 (m, 6H), 3.07-3.15 (m, 1H), 3.24-3.36 (m, 6H), 3.45 (t, J = 5.9 Hz, 2H), 3.51-3.58 (m, 2H), 3.98- 4.02 (m, 2H), 4.07-4.11 (br m, 2H), 7.71 (s, 1H). |
| 192 | 1 | 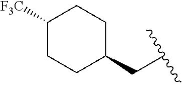 [SMILES: *C[C@H]1CC[C@H](C(F)(F)F)CC1] | 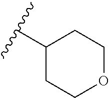 [SMILES: *C1CCOCC1] | (400 MHz, CDCl₃): δ 1.01-1.12 (m, 2H), 1.25-1.35 (m, 2H), 1.90- 2.18 (m, 12H), 3.24-3.32 (m, 1H), 3.35 (d, J = 6.6 Hz, 2H), 3.45 (t, J = 6.0 Hz, 2H), 3.54 (td, J = 11.6, 2.1 Hz, 2H), 4.02 (t, J = 6.0 Hz, 2H), 4.07-4.12 (m, 2H), 7.72 (s, |
| | | | | 1H). |
| 193 | 1 | 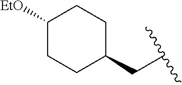 [SMILES: *C[C@H]1CC[C@H](OCC)CC1] | 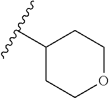 [SMILES: *C1CCOCC1] | (400 MHz, CDCl₃): δ 1.01-1.11 (m, 2H), 1.16-1.26 (m, 2H), 1.20 (t, J = 6.9 Hz, 3H), 1.82-1.94 (m, 5H), 2.05-2.17 (m, 6H), 3.16-3.33 (m, 2H), 3.33 (d, J = 6.9 Hz, 2H), 3.45 (t, J = 6.0 Hz, 2H), 3.50-3.57 (m, 4H), 3.99-4.02 (m, 2H), 4.09 |
| | | | | (dq, J = 11.5, 2.2 Hz, 2H), 7.71 (s, |
| | | | | 1H). |
| 194 | 1 | 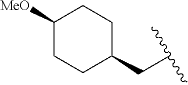 [SMILES: *C[C@H]1CC[C@@H](OC)CC1] | 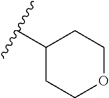 [SMILES: *C1CCOCC1] | (400 MHz, CDCl₃): δ 1.39-1.56 (m, 6H), 1.91-2.15 (m, 9H), 3.26- 3.34 (m, 1H), 3.32 (s, 3H), 3.36 (d, J = 7.1 Hz, 2H), 3.42-3.46 (br m, 1H), 3.45 (t, J = 6.0 Hz, 2H), 3.56 (td, J = 11.7, 2.2 Hz, 2H), 4.01 (t, J = 6.0 Hz, 2H), 4.07-4.11 |
| | | | | (m, 2H), 7.71 (s, 1H). |
| 195 | 1 | 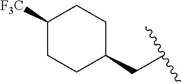 [SMILES: *C[C@H]1CC[C@@H](C(F)(F)F)CC1] | 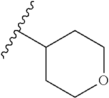 [SMILES: *C1CCOCC1] | (400 MHz, CDCl₃): δ 1.54-1.76 (m, 8H), 1.87-1.92 (m, 2H), 2.03- 2.25 (m, 6H), 3.26-3.35 (m, 1H), 3.42 (t, J = 6.0 Hz, 2H), 3.51-3.58 (m, 4H), 4.02 (t, J = 6.0 Hz, 2H), 4.05-4.10 (m, 2H), 7.73 (s, 1H). |
| 196 | 1 | 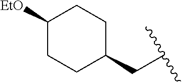 [SMILES: *C[C@H]1CC[C@@H](OCC)CC1] | 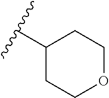 [SMILES: *C1CCOCC1] | (400 MHz, CDCl₃): δ 1.21 (t, J = 7.1 Hz, 3H), 1.41-1.54 (m, 6H), 1.87-2.15 (m, 9H), 3.30 (tt, J = 11.6, 3.9 Hz, 1H), 3.38 (d, J = 7.1 Hz, 2H), 3.43-3.59 (m, 7H), 4.01 (t, J = 6.0 Hz, 2H), 4.06-4.11 (m, 2H), 7.71 (s, 1H). |
| 197 | 1 | 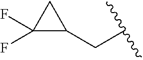 [SMILES: *CC1CC1(F)F] | 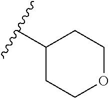 [SMILES: *C1CCOCC1] | (300 MHz, CDCl₃): δ 1.22 (tt, J = 12.5, 4.2 Hz, 1H), 1.52-1.66 (m, 1H), 1.93 (d, J = 8.1 Hz, 2H), 2.07 (tt, J = 15.4, 5.1 Hz, 5H), 3.16 (dd, J = 14.7, 9.5 Hz, 1H), 3.36 (dtd, J = 26.0, 9.4, 4.3 Hz, 2H), 3.58 (td, J = 11.6, 2.4 Hz, |
| | | | | 3H), 4.06 (dtd, J = 39.4, 12.1, 6.8 |
| | | | | Hz, 5H), 7.74 (s, 1H). |

### Examples 198 to 203

TABLE 19

| No. | Structure | ¹H NMR |
|---|---|---|
| 198 | 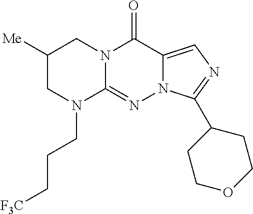 [SMILES: CC1CN(CCCC(F)(F)F)c2nn3c(C4CCOCC4)ncc3c(=O)n2C1] | (400 MHz, CDCl₃): δ 1.14 (d, J = 6.8 Hz, 3H), 1.88-1.91 (m, 2H), 1.98-2.22 (m, 7H), 3.08-3.42 (m, 4H), 3.52-3.59 (m, 4H), 4.07-4.10 (m, 2H), 4.43-4.48 (m, 1H), 7.73 (s, 1H). |
| 199 | 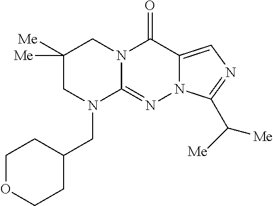 [SMILES: CC(C)c1ncc2c(=O)n3c(nn12)N(CC1CCOCC1)CC(C)(C)C3] | (400 MHz, DMSO-d₆): δ 1.01 (s, 6H), 1.22-1.32 (m, 8H), 1.58-1.61 (m, 2H), 2.08-2.15 (m, 1H), 3.17 (s, 2H), 3.21- 3.30 (m, 3H), 3.34-3.38 (m, 2H), 3.62 (s, 2H), 3.83-3.87 (m, 2H), 7.51 (s, 1H). |
| 200 | 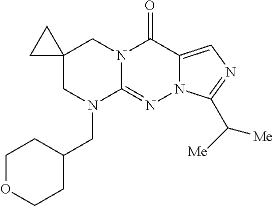 [SMILES: CC(C)c1ncc2c(=O)n3c(nn12)N(CC1CCOCC1)CC1(CC1)C3] | (400 MHz, DMSO-d₆): δ 0.64-0.69 (m, 4H), 1.27-1.31 (m, 8H), 1.58-1.61 (m, 2H), 2.06-2.15 (m, 1H), 3.23-3.25 (m, 2H), 3.28-3.31 (m, 3H), 3.34-3.37 (m, 2H), 3.73 (s, 2H), 3.84-3.87 (m, 2H), 7.52 (s, 1H). |
| 201 | 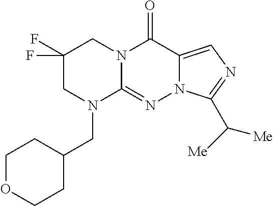 [SMILES: CC(C)c1ncc2c(=O)n3c(nn12)N(CC1CCOCC1)CC(F)(F)C3] | (400 MHz, CDCl₃): δ 1.39-1.48 (m, 8H), 1.66-1.70 (m, 2H), 2.14-2.21 (m, 1H), 3.36-3.44 (m, 5H), 3.72 (t, J = 12.0 Hz, 2H), 4.01-4.05 (m, 2H), 4.29 (t, J = 12.4 Hz, 2H), 7.79 (d, J = 2.0 Hz, 1H). |
| 202 | 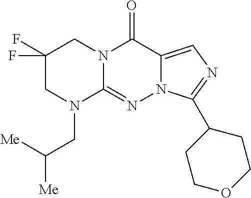 [SMILES: CC(C)CN1CC(F)(F)Cn2c1nn1c(C3CCOCC3)ncc1c2=O] | (400 MHz,CDCl₃): δ 1.01 (d, J = 6.4 Hz, 6H), 1.91-1.95 (m, 2H), 2.06-2.24 (m, 3H), 3.29-3.36 (m, 3H), 3.54-3.61 (m, 2H), 3.72 (t, J = 12.4 Hz, 2H), 4.10-4.12 (m, 2H), 4.29 (t, J = 12.4 Hz, 2H), 7.79 (s, 1H). |
| 203 | 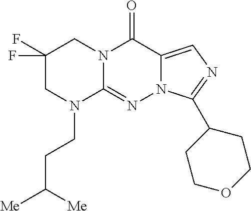 [SMILES: CC(C)CCN1CC(F)(F)Cn2c1nn1c(C3CCOCC3)ncc1c2=O] | (400 MHz,CDCl₃): δ 1.01 (d, J = 6.4 Hz, 6H), 1.62-1.71 (m, 3H), 1.92-1.96 (m, 2H), 2.05-2.16 (m, 2H), 3.30-3.38 (m, 1H), 3.52-3.59 (m, 4H), 3.69 (t, J = 12.4 Hz, 2H), 4.08-4.13 (m, 2H), 4.28 (t, J = 12.0 Hz, 2H), 7.79 (s, 1H). |

### Examples 204 to 229

TABLE 20-1

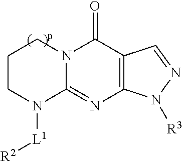

| No. | p | R²—L¹— | R³— | ¹H NMR |
|---|---|---|---|---|
| 204 | 0 | 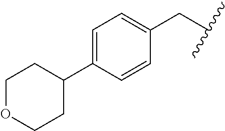 [SMILES: *Cc1ccc(C2CCOCC2)cc1] | 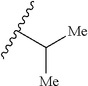 [SMILES: *C(C)C] | (400 MHz, CDCl₃): δ 1.47 (d, J = 6.8 Hz, 6H), 1.48-1.79 (m, 4H), 2.69-2.77 (m, 1H), 3.45-3.57 (m, 4H), 4.02-4.06 (m, 4H), 4.57 (s, 2H), 4.81-4.88 (m, 1H), 7.18-7.26 (m, 4H), 7.86 (s, 1H). |
| 205 | 0 | 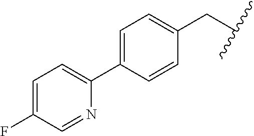 [SMILES: *Cc1ccc(-c2ccc(F)cn2)cc1] | 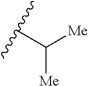 [SMILES: *C(C)C] | (400 MHz, CDCl₃): δ 1.50 (d, J = 6.6 Hz, 6H), 3.57-3.61 (m, 2H), 4.06-4.11 (m, 2H), 4.67 (s, 2H), 4.85-4.92 (m, 1H), 7.41-7.49 (m, 3H), 7.70 (dd, J = 8.8, 4.4 Hz, 1H), 7.90-7.94 (m, 3H), 8.52 (d, J = 2.9 Hz, 1H). |
| 206 | 0 | 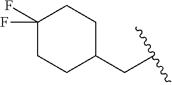 [SMILES: *CC1CCC(F)(F)CC1] | 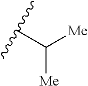 [SMILES: *C(C)C] | (300 MHz, CDCl₃): δ 1.46 (dd, J = 18.0, 9.2 Hz, 6H), 1.73 (dd, J = 35.2, 16.9 Hz, 7H), 2.14 (s, 2H), 3.36 (d, J = 6.6 Hz, 2H), 3.72 (t, J = 8.4 Hz, 2H), 4.13 (t, J = 8.4 Hz, 2H), 4.78-4.87 (m, 1H), 7.89 (s, |
| | | | | 1H). |
| 207 | 0 | 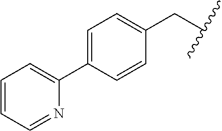 [SMILES: *Cc1ccc(-c2ccccn2)cc1] | 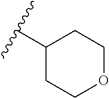 [SMILES: *C1CCOCC1] | (400 MHz, CDCl₃): δ 1.88-1.91 (m, 2H), 2.36 (ddd, J = 24.9, 12.3, 4.5 Hz, 2H), 3.56-3.60 (m, 4H), 4.07-4.13 (m, 4H), 4.63-4.69 (m, 3H), 7.23-7.25 (m, 1H), 7.42 (d, J = 8.3 Hz, 2H), 7.70-7.77 (m, 2H), 7.90 (s, 1H), 7.98-8.00 (m, 2H), 8.68-8.69 (m, 1H). |
| 208 | 0 | 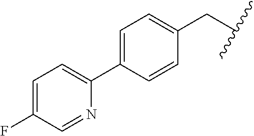 [SMILES: *Cc1ccc(-c2ccc(F)cn2)cc1] | 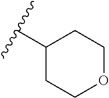 [SMILES: *C1CCOCC1] | (400 MHz, CDCl₃): δ 1.89-1.94 (m, 2H), 2.38 (dt, J = 12.4, 4.3 Hz, 2H), 3.55-3.64 (m, 4H), 4.09- 4.15 (m, 4H), 4.64-4.71 (m, 3H), 7.43 (d, J = 8.0 Hz, 2H), 7.49 (td, J = 8.4, 2.8 Hz, 1H), 7.72 (dd, J = 8.9, 4.3 Hz, 1H), 7.92 (s, 1H), 7.95 (d, J = 8.0 Hz, 2H), 8.55 (d, J = 2.9 Hz, 1H). |
| 209 | 0 | 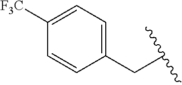 [SMILES: *Cc1ccc(C(F)(F)F)cc1] | 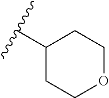 [SMILES: *C1CCOCC1] | (400 MHz, CDCl₃): δ 1.89 (dq, J = 12.5, 2.0 Hz, 2H), 2.37 (dt, J = 12.5, 4.4 Hz, 2H), 3.53-3.64 (m, 4H), 4.11-4.15 (m, 4H), 4.65 (tt, J = 11.6, 4.4 Hz, 1H), 4.71 (s, 2H), 7.46 (d, J = 7.8 Hz, 2H), 7.65 (d, J = 7.8 Hz, 2H), 7.92 (s, 1H). |
| 210 | 0 | 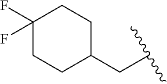 [SMILES: *CC1CCC(F)(F)CC1] | 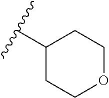 [SMILES: *C1CCOCC1] | (300 MHz, CDCl₃): δ 1.46 (t, J = 12.1 Hz, 2H), 1.62-1.89 (m, 7H), 2.15 (s, 2H), 2.34 (ddd, J = 24.8, 12.3, 4.6 Hz, 2H), 3.37 (d, J = 6.6 Hz, 2H), 3.59 (dd, J = 17.2, 7.0 Hz, 2H), 3.72 (t, J = 8.4 Hz, 2H), 4.14 (t, J = 8.4 Hz, 4H), 4.56-4.67 |
| | | | | (m, 1H), 7.89 (s, 1H). |
| 211 | 0 | 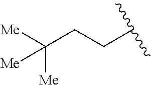 [SMILES: *CCC(C)(C)C] | 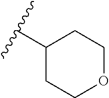 [SMILES: *C1CCOCC1] | (300 MHz, CDCl₃): δ 1.01 (s, 9H), 1.51-1.56 (m, 1H), 1.65 (d, J = 12.5 Hz, 1H), 1.90 (dd, J = 12.5, 2.2 Hz, 2H), 2.35 (ddd, J = 24.9, 12.5, 4.4 Hz, 2H), 3.44-3.60 (m, 4H), 3.71 (t, J = 8.4 Hz, 2H), 4.11 (dd, J = 14.7, 6.6 Hz, 4H), |
| | | | | 4.57-4.66 (m, 1H), 7.88 (s, 1H). |
| 212 | 0 | 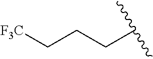 [SMILES: *CCCC(F)(F)F] | 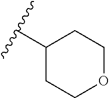 [SMILES: *C1CCOCC1] | (300 MHz, CDCl₃): δ 1.93 (tt, J = 18.7, 6.2 Hz, 4H), 2.15-2.41 (m, 4H), 3.57 (td, J = 13.0, 4.2 Hz, 4H), 3.73 (dd, J = 10.6, 7.0 Hz, 2H), 4.14 (q, J = 7.3 Hz, 4H), 4.62 (tt, J = 11.7, 4.3 Hz, 1H), 7.89 (s, 1H). |
| 213 | 0 | 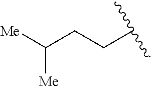 [SMILES: *CCC(C)C] | 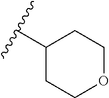 [SMILES: *C1CCOCC1] | (300 MHz, CDCl₃): δ 0.99 (d, J = 6.6 Hz, 6H), 1.52 (dd, J = 14.3, 7.0 Hz, 2H), 1.66 (td, J = 11.9, 5.9 Hz, 1H), 1.88 (dd, J = 12.5, 2.2 Hz, 2H), 2.35 (ddd, J = 24.9, 12.5, 4.4 Hz, 2H), 3.54 (tt, J = 18.7, 6.4 Hz, 4H), 3.70 (t, J = 8.4 Hz, 2H), |
| | | | | 4.11 (dd, J = 10.3, 6.6 Hz, 4H), |
| | | | | 4.62 (tt, J = 11.4, 4.0 Hz, 1H), |
| | | | | 7.88 (s, 1H). |
| 214 | 1 | 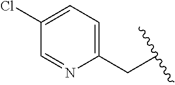 [SMILES: *Cc1ccc(Cl)cn1] | 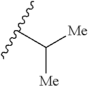 [SMILES: *C(C)C] | (400 MHz, CDCl₃): δ 1.42 (d, J = 6.9 Hz, 6H), 2.07-2.13 (m, 2H), 3.62 (t, J = 6.0 Hz, 2H), 4.10 (t, J = 6.0 Hz, 2H), 4.67-4.76 (m, 1H), 4.94 (s, 2H), 7.33 (d, J = 8.6 Hz, 1H), 7.63 (dd, J = 8.6, 2.4 Hz, |
| | | | | 1H), 7.88 (s, 1H), 8.52 (d, J = 2.4 |
| | | | | Hz, 1H). |
| 215 | 1 | 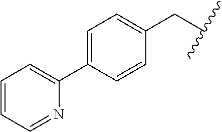 [SMILES: *Cc1ccc(-c2ccccn2)cc1] | 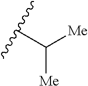 [SMILES: *C(C)C] | (300 MHz, CDCl₃): δ 1.45 (t, J = 9.5 Hz, 6H), 2.02-2.10 (m, 2H), 3.46 (q, J = 6.4 Hz, 2H), 4.09 (t, J = 5.9 Hz, 2H), 4.75-4.88 (m, 1H), 4.99 (s, 2H), 7.32 (td, J = 5.0, 2.4 Hz, 1H), 7.46 (t, J = 9.5 Hz, 2H), 7.75 (d, J = 8.1 Hz, 1H), 7.85 (td, J = 7.7, 1.5 Hz, 1H), 7.94 (t, J = |
| | | | | 8.4 Hz, 3H), 8.76 (d, J = 5.1 Hz, |
| | | | | 1H). |
| 216 | 1 | 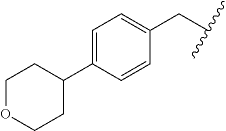 [SMILES: *Cc1ccc(C2CCOCC2)cc1] | 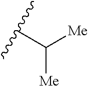 [SMILES: *C(C)C] | (300 MHz, CDCl₃): δ 1.44 (t, J = 11.0 Hz, 6H), 1.69-1.88 (m, 4H), 2.00-2.07 (m, 2H), 2.70-2.81 (m, 1H), 3.48 (tt, J = 21.3, 5.9 Hz, 4H), 4.07 (t, J = 5.9 Hz, 4H), 4.80 (td, J = 13.4, 6.8 Hz, 1H), 4.90 (s, 2H), 7.19-7.32 (m, 4H), 7.90 (s, 1H). |
| 217 | 1 | 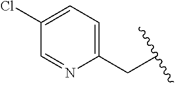 [SMILES: *Cc1ccc(Cl)cn1] | 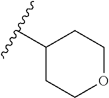 [SMILES: *C1CCOCC1] | (400 MHz, CDCl₃): δ 1.75 (dq, J = 12.8, 2.0 Hz, 2H), 2.09-2.15 (m, 2H), 2.26 (dt, J = 12.2, 6.1 Hz, 2H), 3.49 (td, J = 12.2, 2.0 Hz, 2H), 3.65 (t, J = 6.1 Hz, 2H), 4.05-4.12 (m, 4H), 4.46 (tt, J = 11.7, 4.1 Hz, 1H), 4.92 (s, 2H), |
| | | | | 7.30 (d, J = 8.4 Hz, 1H), 7.63 (dd, |
| | | | | J = 8.4, 2.2 Hz, 1H), 7.87 (s, 1H), |
| | | | | 8.52 (d, J = 2.2 Hz, 1H). |
| 218 | 1 | 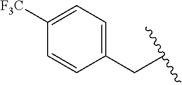 [SMILES: *Cc1ccc(C(F)(F)F)cc1] | 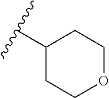 [SMILES: *C1CCOCC1] | (400 MHz, CDCl₃): δ 1.78 (dq, J = 12.6, 1.8 Hz, 2H), 2.07-2.13 (m, 2H), 2.27 (dt, 12.4, 4.3 Hz, 2H), 3.45-3.51 (m, 4H), 4.03-4.08 (m, 2H), 4.09-4.12 (m, 2H), 4.50 (tt, J = 11.6, 4.3 Hz, 1H), 4.96 (s, 2H), |
| | | | | 7.44 (d, J = 7.8 Hz, 2H), 7.61 (d, |
| | | | | J = 8.0 Hz, 2H), 7.90 (s, 1H). |
| 219 | 1 | 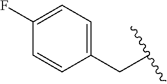 [SMILES: *Cc1ccc(F)cc1] | 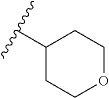 [SMILES: *C1CCOCC1] | (400 MHz, CDCl₃): δ 1.79-1.88 (m, 2H), 2.01-2.09 (m, 2H), 2.25- 2.39 (m, 2H), 3.42 (t, J = 6.0 Hz, 2H), 3.48-3.57 (m, 2H), 4.04-4.13 (m, 4H), 4.51-4.62 (m, 1H), 4.88 (s, 2H), 6.99-7.07 (m, 2H), 7.29- 7.35 (m, 2H), 7.90 (s, 1H). |
| 220 | 1 | 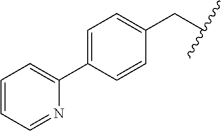 [SMILES: *Cc1ccc(-c2ccccn2)cc1] | 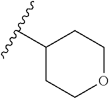 [SMILES: *C1CCOCC1] | (400 MHz, CDCl₃): δ 1.81-1.84 (m, 2H), 2.03-2.06 (m, 2H), 2.30 (ddd, J = 24.8, 12.1, 4.7 Hz, 2H), 3.44 (t, J = 6.0 Hz, 2H), 3.50 (td, J = 12.0, 2.0 Hz, 2H), 4.03-4.09 (m, 4H), 4.55-4.57 (m, 1H), 4.96 (s, 2H), 7.21-7.24 (m, 1H), 7.42 (d, J = 8.5 Hz, 2H), 7.70-7.74 (m, |
| | | | | 2H), 7.89 (s, 1H), 7.95-7.96 (m, |
| | | | | 2H), 8.66-8.68 (m, 1H). |
| 221 | 1 | 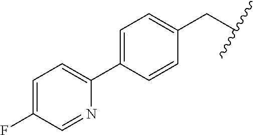 [SMILES: *Cc1ccc(-c2ccc(F)cn2)cc1] | 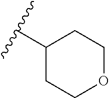 [SMILES: *C1CCOCC1] | (400 MHz, CDCl₃): δ 1.84 (dq, J = 12.7, 1.9 Hz, 2H), 2.04-2.10 (m, 2H), 2.32 (dt, J = 12.4, 4.6 Hz, 2H), 3.46 (t, J = 6.0 Hz, 2H), 3.52 (td, J = 11.9, 1.9 Hz, 2H), 4.05- 4.11 (m, 4H), 4.53-4.62 (m, 1H), 4.97 (s, 2H), 7.42-7.52 (m, 3H), 7.71 (dd, J = 8.5, 3.9 Hz, 1H), 7.92 (dd, J = 6.9, 2.6 Hz, 3H), |
| | | | | 8.54 (d, J = 2.6 Hz, 1H). |
| 222 | 1 | 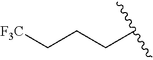 [SMILES: *CCCC(F)(F)F] | 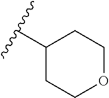 [SMILES: *C1CCOCC1] | (300 MHz, CDCl₃): δ 1.88 (dd, J = 12.5, 2.2 Hz, 2H), 1.96-2.43 (m, 8H), 3.53 (ddd, J = 23.8, 11.4, 2.9 Hz, 4H), 3.72 (t, J = 7.3 Hz, 2H), 4.04-4.15 (m, 4H), 4.58 (tt, J = 11.4, 4.0 Hz, 1H), 7.89 (s, 1H). |
| 223 | 1 | 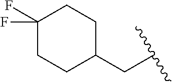 [SMILES: *CC1CCC(F)(F)CC1] | 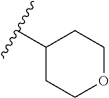 [SMILES: *C1CCOCC1] | (300 MHz, CDCl₃): δ 1.46 (t, J = 12.1 Hz, 2H), 1.62-1.91 (m, 6H), 2.11 (dt, J = 20.1, 7.7 Hz, 5H), 2.39 (ddd, J = 24.9, 12.5, 4.4 Hz, 2H), 3.47-3.60 (m, 6H), 4.04-4.14 (m, 4H), 4.55 (dt, J = 12.7, 4.8 Hz, 1H), 7.87 (s, 1H). |
| 224 | 1 | 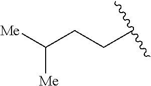 [SMILES: *CCC(C)C] | 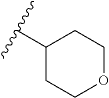 [SMILES: *C1CCOCC1] | (300 MHz, CDCl₃): δ 1.00 (d, J = 5.9 Hz, 6H), 1.61 (dq, J = 25.7, 6.8 Hz, 3H), 1.90 (t, J = 6.6 Hz, 2H), 2.02-2.10 (m, 2H), 2.36 (ddd, J = 24.6, 12.1, 4.4 Hz, 2H), 3.50 (dt, J = 22.3, 9.0 Hz, 4H), 3.66 (t, J = 7.7 Hz, 2H), 4.02-4.14 |
| | | | | (m, 4H), 4.59 (ddd, J = 17.6, 9.9, |
| | | | | 4.0 Hz, 1H), 7.87 (s, 1H). |
| 225 | 1 | 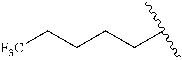 [SMILES: *CCCCC(F)(F)F] | 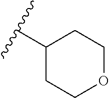 [SMILES: *C1CCOCC1] | (300 MHz, CDCl₃): δ 1.60-1.71 (m, 2H), 1.83 (ddd, J = 26.6, 13.8, 5.0 Hz, 4H), 2.04-2.44 (m, 6H), 3.52 (tt, J = 17.6, 6.1 Hz, 4H), 3.69 (t, J = 7.0 Hz, 2H), 4.04-4.15 (m, 4H), 4.52-4.60 (m, 1H), 7.88 (s, 1H). |
| 226 | 1 | 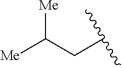 [SMILES: *CC(C)C] | 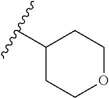 [SMILES: *C1CCOCC1] | (300 MHz, CDCl₃): δ 0.98 (d, J = 6.6 Hz, 6H), 1.90 (dd, J = 12.5, 2.2 Hz, 2H), 2.03-2.44 (m, 5H), 3.47-3.60 (m, 6H), 4.03-4.15 (m, 4H), 4.56 (tt, J = 11.7, 4.2 Hz, 1H), 7.87 (s, 1H). |
| 227 | 1 | 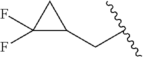 [SMILES: *CC1CC1(F)F] | 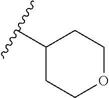 [SMILES: *C1CCOCC1] | (300 MHz, CDCl₃): δ 1.20-1.28 (m, 1H), 1.51-1.63 (m, 1H), 2.01 (tt, J = 33.4, 11.7 Hz, 5H), 2.29- 2.41 (m, 2H), 3.23 (dd, J = 14.3, 7.7 Hz, 1H), 3.52 (tt, J = 27.1, 9.0 Hz, 4H), 4.04-4.15 (m, 4H), 4.36 |
| | | | | (td, J = 9.5, 5.1 Hz, 1H), 4.56- |
| | | | | 4.64 (m, 1H), 7.90 (s, 1H). |
| 228 | 1 | 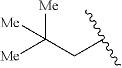 [SMILES: *CC(C)(C)C] | 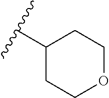 [SMILES: *C1CCOCC1] | (300 MHz, CDCl₃): δ 1.03 (s, 9H), 1.87 (dd, J = 12.5, 2.2 Hz, 2H), 2.02-2.10 (m, 2H), 2.35 (ddd, J = 24.8, 11.9, 4.6 Hz, 2H), 3.54 (dd, J = 7.0, 4.0 Hz, 4H), 3.59 (d, J = 3.7 Hz, 2H), 4.04- 4.15 (m, 4H), 4.53-4.61 (m, 1H), |
| | | | | 7.88 (s, 1H). |
| 229 | 1 | 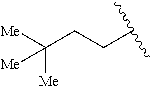 [SMILES: *CCC(C)(C)C] | 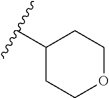 [SMILES: *C1CCOCC1] | (400 MHz, CDCl₃): δ 1.01 (s, 9H), 1.50-1.63 (m, 2H), 1.86-1.94 (m, 2H), 2.00-2.11 (m, 2H), 2.27- 2.42 (m, 2H), 3.41-3.55 (m, 4H), 3.61-3.70 (m, 2H), 4.00-4.07 (m, 2H), 4.08-4.16 (m, 2H), 4.54-4.65 (m, 1H), 7.87 (s, 1H). |

The compounds of Examples 230 to 233 were synthesized in a similar manner to Examples 84 and 85.

TABLE 21

| | | | SFC: |
|---|---|---|---|
| | | | retention |
| | | | time/ |
| No. | Structure | ¹H NMR | Method |
| 230 | 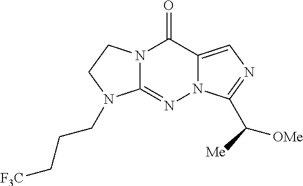 [SMILES: CO[C@@H](C)c1ncc2c(=O)n3c(nn12)N(CCCC(F)(F)F)CC3] | (400 MHz, CDCl₃): δ 1.66 (d, J = 5.6 Hz, 3H), 1.96- 2.02 (m, 2H), 2.22-2.27 (m, 2H), 3.33 (s, 3H), 3.46-3.49 (m, 2H), 3.70- 3.73 (m, 2H), 4.13-4.17 (m, 2H), 4.88-4.92 (m, 1H), 7.81 (s, 1H). | 7.03 min./ Method G |
| 231 | 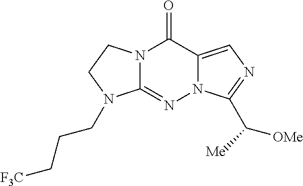 [SMILES: CO[C@H](C)c1ncc2c(=O)n3c(nn12)N(CCCC(F)(F)F)CC3] | (400 MHz, CDCl₃): δ 1.66 (d, J = 5.6 Hz, 3H), 1.96- 2.02 (m, 2H), 2.22-2.27 (m, 2H), 3.33 (s, 3H), 3.46-3.49 (m, 2H), 3.70- 3.73 (m, 2H), 4.13-4.17 (m, 2H), 4.88-4.92 (m, 1H), 7.81 (s, 1H). | 7.95 min./ Method G |
| 232 | 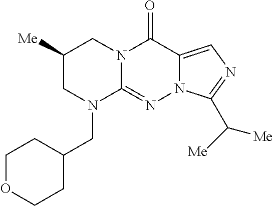 [SMILES: CC(C)c1ncc2c(=O)n3c(nn12)N(CC1CCOCC1)C[C@@H](C)C3] | (400 MHz, CDCl₃): δ 1.13 (4 J = 6.8 Hz, 3H), 1.38 (d, J = 7.2 Hz, 6H), 1.40- 1.46 (m, 2H), 1.64-1.66 (m, 2H), 2.17-2.23 (m, 2H), 3.10-3.21 (m, 2H), 3.32-3.48 (m, 6H), 4.00- 4.03 (m, 2H), 4.43-4.48 (m, 1H), 7.72 (s, 1H). | 4.61 min./ Method H |
| 233 | 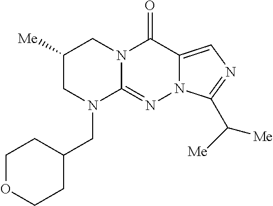 [SMILES: CC(C)c1ncc2c(=O)n3c(nn12)N(CC1CCOCC1)C[C@H](C)C3] | (400 MHz, CDCl₃): δ 1.13 (d, J = 6.8 Hz, 3H), 1.38 (d, J = 7.2 Hz, 6H), 1.40- 1.46 (m, 2H), 1.64-1.66 (m, 2H), 2.17-2.23 (m, 2H), 3.10-3.21 (m, 2H), 3.32-3.48 (m, 6H), 4.00- 4.03 (m, 2H), 4.43-4.48 (m, 1H), 7.72 (s, 1H). | 5.14 min./ Method H |

### Example 234

### 6-Methyl-1-(3-methylbutyl)-8-(tetrahydro-2H-pyran-4-yl)-2,3-dihydro-1H,5H-diimidazo[2,1-c:5′,1′-f][1,2,4]triazin-5-one

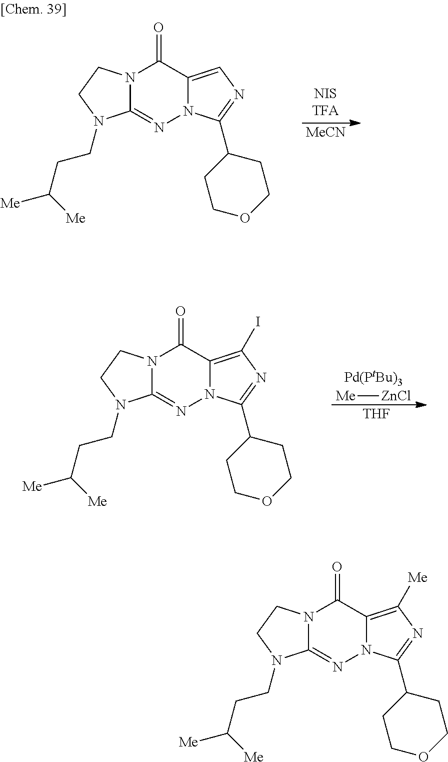

To a solution of 6-iodo-1-(3-methylbutyl)-8-(tetrahydro-2H-pyran-4-yl)-2,3-dihydro-1H,5H-diimidazo[2,1-c:5′,1′-f][1,2,4]triazin-5-one (30.0 mg, 0.066 mmol) in THF (0.5 mL) was added Pd(P^{t}Bu₃)₂(6.7 mg, 0.013 mmol) and MeZnCl (2 M in THF solution, 0.26 mL). The mixture was stirred at room temperature under nitrogen atmosphere. After 3 h, H₂O was added to the reaction mixture. The aqueous layer was extracted with EtOAc, and the combined organic phases were dried with sodium sulfate and concentrated to dryness. The residue was purified by silica gel column chromatography (hexane/EtOAc) to give the titled compound (18.5 mg, 82%) as a white solid. ¹H NMR (400 MHz, CDCl₃): δ 0.96 (d, J=6.6 Hz, 6H), 1.58 (m, 3H), 1.86 (m, 2H), 2.05 (m, 2H), 2.54 (s, 3H), 3.26 (m, 1H), 3.33 (t, J=7.3 Hz, 2H), 3.52 (m, 2H), 3.60 (m, 2H), 4.04 (m, 4H).

### 6-Iodo-1-(3-methylbutyl)-8-(tetrahydro-2H-pyran-4-yl)-2,3-dihydro-1H,5H-diimidazo[2,1-c:5′,1′-f][1,2,4]triazin-5-one

1-(3-methylbutyl)-8-(tetrahydro-2H-pyran-4-yl)-2,3-dihydro-1H,5H-diimidazo[2,1-c: 5′,1′-f][1,2,4]triazin-5-one (100.0 mg, 0.302 mmol) in TFA (0.11 mL) and MeCN (1.5 mL) was added NIS (100.0 mg, 0.45 mmol). The mixture was stirred at room temperature under nitrogen atmosphere. After 3 h, saturated NaHCO₃ solution was added to the reaction mixture. The aqueous layer was extracted with EtOAc, and the combined organic phases were dried with sodium sulfate and concentrated to dryness. The residue was purified by silica gel column chromatography (hexane/EtOAc) to give the titled compound (138 mg, 100%) as a red solid. ¹H NMR (400 MHz, CDCl₃): δ 0.96 (d, J=6.6 Hz, 6H), 1.48-1.66 (m, 3H), 1.83-1.86 (m, 2H), 2.01-2.11 (m, 2H), 3.24-3.35 (m, 3H), 3.48-3.54 (m, 3H), 3.62-3.66 (m, 1H), 4.02-4.06 (m, 4H).

### Example 235

### 1-(3-Methylbutyl)-5-oxo-8-(tetrahydro-2H-pyran-4-yl)-2,3-dihydro-1H,5H-diimidazo[2,1-c 5′,1′-f][1,2,4]triazine-6-carbonitrile

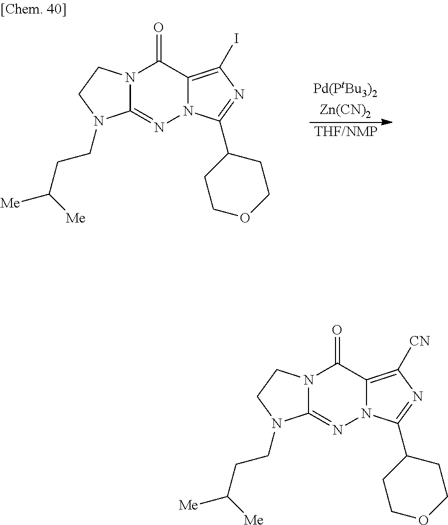 [SMILES: CC(C)CCN1CCn2c1nn1c(C3CCOCC3)nc(I)c1c2=O ; "THF/NMP" ; CC(C)CCN1CCn2c1nn1c(C3CCOCC3)nc(C#N)c1c2=O]

To a solution of 6-iodo-1-(3-methylbutyl)-8-(tetrahydro-2H-pyran-4-yl)-2,3-dihydro-1H,5H-diimidazo[2,1-c:5′,1′-f][1,2,4]triazin-5-one (24.0 mg, 0.053 mmol) in THF (0.8 mL) and NMP (0.4 mL) was added Pd(PBu₃)₂(5.4 mg, 0.011 mmol) and Zn(CN)₂ (12.3 mg, 0.11 mmol). The mixture was stirred at 80° C. under nitrogen atmosphere overnight. Saturated NaHCO₃ solution was added to the reaction mixture. The aqueous layer was extracted with EtOAc, and the combined organic phases were dried with sodium sulfate and concentrated to dryness. The residue was purified by silica gel column chromatography (hexane/EtOAc) to give the titled compound (1.5 mg, 8%) as a white solid. ¹H NMR (400 MHz, CDCl₃): δ 0.97 (d, J=6.3 Hz, 6H), 1.52 (m, 2H), 1.63 (m, 1H), 1.89 (m, 2H), 2.02 (m, 2H), 3.29 (m, 1H), 3.37 (t, J=7.4 Hz, 2H), 3.53 (m, 2H), 3.71 (t, J=8.2 Hz, 2H), 4.05 (m, 2H), 4.13 (m, 2H).

### Example 236

### 6-Chloro-1-(3-methylbutyl)-8-(tetrahydro-2H-pyran-4-yl)-2,3-dihydro-1H,5H-diimidazo[2,1-c:5′,1′-f][1,2,4]triazin-5-one

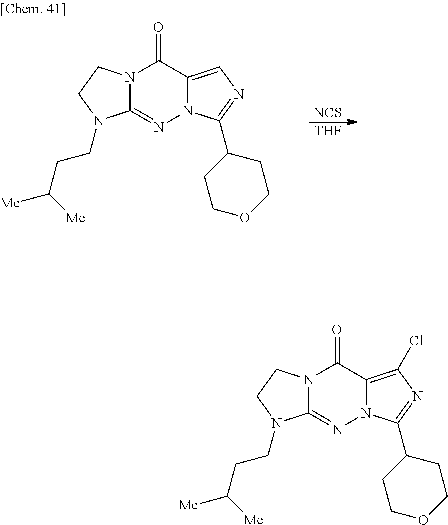 [SMILES: CC(C)CCN1CCn2c1nn1c(C3CCOCC3)ncc1c2=O ; "NCS" ; CC(C)CCN1CCn2c1nn1c(C3CCOCC3)nc(Cl)c1c2=O ; "THF"]

To a solution of 1-(3-methylbutyl)-8-(tetrahydro-2H-pyran-4-yl)-2,3-dihydro-1H,5H-diimidazo[2,1-c:5′,1′-f][1,2,4]triazin-5-one (20.0 mg, 0.060 mmol) in THF (0.5 mL) was added NCS (16.0 mg, 0.12 mmol). The mixture was stirred at room temperature under nitrogen atmosphere. After 3 h, saturated NaHCO₃ solution was added to the reaction mixture. The aqueous layer was extracted with EtOAc, and the combined organic phases were dried with sodium sulfate and concentrated to dryness. The residue was purified by silica gel column chromatography (hexane/EtOAc) to give the titled compound (9.2 mg, 42%) as a white solid. ¹H NMR (400 MHz, CDCl₃): δ 0.92 (d, J=6.6 Hz, 6H), 1.47 (m, 2H), 1.55-1.63 (m, 1H), 1.81 (m, 2H), 1.98 (m, 2H), 3.17-3.31 (m, 3H), 3.47 (td, J=11.4, 2.4 Hz, 2H), 3.59 (dd, J=9.9, 6.2 Hz, 2H), 4.01 (m, 4H).

In Vitro HTRF PDE1 Inhibition Assay

An exemplary procedure for the in vitro Homogenous Time Resolved Fluorescence assay, which can be used to determine the inhibitory action of compounds of the invention toward PDE1 or its isoforms, follows.

The HTRF PDE1 assay utilized the HTRF (Homogenous Time Resolved Fluorescence) technology, which is based on the competition between unlabeled cyclic nucleotide and cyclic nucleotide labeled with XL665 for the binding to cyclic nucleotide-specific antibody labeled with cryptate. The HTRF signal is thus inversely proportional to the concentration of cyclic nucleotide being measured. Since phosphodiesterases break down cyclic nucleotides the HTRF signal was used to determine PDE activity.

The Cisbio cGMP HTRF assay kit (Cat no: 62GM2PEC) was utilized. Cyclic GMP was diluted to 200 nM in HTRF assay buffer (1 mM CaCl₂, 10 mM MgCl₂, 10 mM Tris-HCl, 0.1% BSA, pH 7.4). 10 μl of compound or DMSO was diluted in 200 nM cyclic GMP solution and added to wells of a 96 well white plate to give 100 nM cyclic GMP in 1% DMSO final concentration. PDE (1A3, 1B or IC) was diluted to 2× working concentration in HTRF assay buffer with 2 μg/ml calmodulin, and 10 μl was added to initiate the reaction. The plate was then incubated for 45 minutes at 37° C. d2-Labelled cyclic GMP and anti-cGMP cryptate were diluted in 50 mM phosphate buffer, 0.8 M KF, 1% Triton X100, 0.2% BSA, pH 7.0. Following incubation 10 μl d2-cGMP, then 10 μl anti cGMP cryptate were added to each well and the plate was incubated for 45 minutes at room temperature. The plate was then read on Perkin Elmer Victor at 2 different FRET readings ex/emm 340 nm/665 nm and 340 nm/615 nm.

Data obtained from the HTRF assay for selected compounds of the invention are listed in Tables below. Compounds having an IC₅₀ of <1 μM, are denoted as +++. Compounds having an IC₅₀ of 1−10 μM, are denoted as ++. Compounds having an IC₅₀ of 10-100 μM, are denoted as +.

TABLE 22
Table 1: Results of in vitro
PDE1BHTRF Assay

| Example | IC₅₀ |
|---|---|
| No. | (μM) |
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | ++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 39 | +++ |
| 40 | +++ |
| 41 | +++ |
| 42 | +++ |
| 43 | +++ |
| 44 | +++ |
| 45 | +++ |
| 46 | +++ |
| 47 | +++ |
| 48 | +++ |
| 49 | +++ |
| 50 | ++ |
| 51 | +++ |
| 52 | +++ |
| 53 | +++ |
| 54 | +++ |
| 55 | +++ |
| 56 | +++ |
| 57 | +++ |
| 58 | +++ |
| 59 | +++ |
| 60 | +++ |
| 61 | ++ |
| 62 | +++ |
| 63 | +++ |
| 64 | +++ |
| 65 | +++ |
| 66 | +++ |
| 67 | +++ |
| 68 | +++ |
| 69 | +++ |
| 70 | +++ |
| 71 | +++ |
| 72 | +++ |
| 73 | +++ |
| 74 | +++ |
| 75 | +++ |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | ++ |
| 88 | +++ |
| 89 | ++ |
| 90 | +++ |
| 91 | ++ |
| 92 | ++ |
| 93 | + |
| 94 | +++ |
| 95 | ++ |
| 96 | ++ |
| 97 | ++ |
| 98 | ++ |
| 99 | ++ |
| 100 | +++ |
| 101 | +++ |
| 102 | +++ |
| 103 | +++ |
| 104 | +++ |
| 105 | +++ |
| 106 | +++ |
| 107 | ++ |
| 108 | +++ |
| 109 | +++ |
| 110 | +++ |
| 111 | +++ |
| 112 | ++ |
| 113 | ++ |
| 114 | +++ |
| 115 | +++ |
| 116 | +++ |
| 117 | + |
| 118 | +++ |
| 119 | +++ |
| 120 | +++ |
| 121 | +++ |
| 122 | +++ |
| 123 | ++ |
| 124 | + |
| 125 | ++ |
| 126 | +++ |
| 127 | +++ |
| 128 | ++ |
| 129 | +++ |
| 130 | +++ |
| 131 | +++ |
| 132 | +++ |
| 133 | +++ |
| 134 | ++ |
| 135 | ++ |
| 136 | ++ |
| 137 | +++ |
| 138 | ++ |

TABLE 23
Table 2: Results of in vitro
PDE1A3 HTRF Assay

| Example | IC₅₀ |
|---|---|
| No. | (μM) |
| 1 | +++ |
| 9 | +++ |
| 11 | +++ |
| 16 | +++ |
| 24 | +++ |
| 25 | +++ |
| 30 | +++ |
| 55 | +++ |
| 56 | ++ |
| 59 | +++ |
| 64 | +++ |
| 66 | +++ |
| 69 | +++ |
| 70 | +++ |
| 75 | +++ |
| 78 | +++ |
| 79 | +++ |
| 81 | +++ |
| 106 | +++ |
| 120 | +++ |
| 137 | ++ |

TABLE 24
Table 3: Results of in
vitro PDE1C HTRF Assay

| Example | IC₅₀ |
|---|---|
| No. | (μM) |
| 1 | ++ |
| 9 | +++ |
| 11 | ++ |
| 16 | +++ |
| 24 | +++ |
| 25 | +++ |
| 30 | +++ |
| 55 | +++ |
| 56 | + |
| 59 | ++ |
| 64 | +++ |
| 66 | ++ |
| 69 | ++ |
| 70 | ++ |
| 75 | +++ |
| 78 | ++ |
| 79 | +++ |
| 81 | ++ |
| 106 | +++ |
| 120 | +++ |
| 137 | ++ |

TABLE 25
Table 4: Results of in vitro
PDE1B HTRF Assay

| Example | IC₅₀ |
|---|---|
| No. | (μM) |
| 139 | +++ |
| 140 | +++ |
| 141 | +++ |
| 142 | +++ |
| 143 | +++ |
| 144 | +++ |
| 145 | +++ |
| 146 | +++ |
| 147 | +++ |
| 148 | +++ |
| 149 | +++ |
| 150 | +++ |
| 151 | +++ |
| 152 | +++ |
| 153 | +++ |
| 154 | +++ |
| 155 | +++ |
| 156 | +++ |
| 157 | +++ |
| 158 | +++ |
| 159 | +++ |
| 160 | +++ |
| 161 | +++ |
| 162 | +++ |
| 163 | +++ |
| 164 | +++ |
| 165 | +++ |
| 166 | +++ |
| 167 | +++ |
| 168 | +++ |
| 169 | +++ |
| 170 | +++ |
| 171 | +++ |
| 172 | +++ |
| 173 | +++ |
| 174 | +++ |
| 175 | +++ |
| 176 | +++ |
| 177 | +++ |
| 178 | +++ |
| 179 | +++ |
| 180 | +++ |
| 181 | ++ |
| 182 | +++ |
| 183 | +++ |
| 184 | +++ |
| 185 | +++ |
| 186 | +++ |
| 187 | +++ |
| 190 | +++ |
| 191 | +++ |
| 192 | +++ |
| 193 | +++ |
| 194 | +++ |
| 195 | +++ |
| 196 | +++ |
| 197 | +++ |
| 198 | +++ |
| 199 | +++ |
| 200 | +++ |
| 201 | +++ |
| 202 | +++ |
| 203 | +++ |
| 204 | +++ |
| 205 | +++ |
| 206 | +++ |
| 207 | +++ |
| 208 | +++ |
| 209 | +++ |
| 210 | +++ |
| 211 | +++ |
| 212 | +++ |
| 213 | +++ |
| 214 | +++ |
| 215 | +++ |
| 216 | +++ |
| 217 | +++ |
| 218 | +++ |
| 219 | +++ |
| 220 | +++ |
| 221 | +++ |
| 222 | +++ |
| 223 | +++ |
| 224 | +++ |
| 225 | +++ |
| 226 | +++ |
| 227 | +++ |
| 228 | +++ |
| 229 | +++ |
| 230 | +++ |
| 231 | ++ |
| 232 | +++ |
| 233 | +++ |
| 234 | ++ |
| 235 | ++ |
| 236 | +++ |

TABLE 26
Table 5: Results of in vitro
PDE1A3 HTRF Assay

| Example | IC₅₀ |
|---|---|
| No. | (μM) |
| 153 | +++ |
| 154 | +++ |
| 155 | +++ |
| 156 | +++ |
| 159 | +++ |
| 160 | +++ |
| 162 | +++ |
| 163 | +++ |
| 164 | +++ |
| 167 | +++ |
| 171 | +++ |
| 172 | +++ |
| 187 | +++ |
| 188 | +++ |
| 190 | +++ |
| 191 | +++ |
| 193 | +++ |
| 200 | +++ |
| 232 | +++ |
| 233 | +++ |

TABLE 27
Table 6: Results of in vitro
PDE1C HTRF Assay

| Example | IC₅₀ |
|---|---|
| No. | (μM) |
| 153 | +++ |
| 154 | +++ |
| 155 | +++ |
| 156 | +++ |
| 159 | +++ |
| 160 | +++ |
| 162 | +++ |
| 163 | +++ |
| 167 | +++ |
| 171 | +++ |
| 172 | +++ |
| 187 | +++ |
| 188 | +++ |
| 190 | +++ |
| 191 | +++ |
| 193 | +++ |
| 200 | +++ |
| 232 | +++ |
| 233 | +++ |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 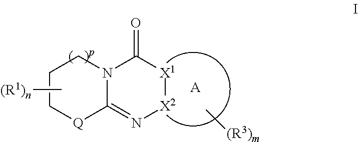
or a pharmaceutically acceptable salt thereof, wherein:
Q is —N(L¹-R²)—, —C(R⁴)₂—, —O—, or —S—;
X¹ and X² are each independently C or N;
Ring A is a 5-6 membered heteroaryl ring;
L is a covalent bond, or a C₁₋₆ bivalent straight or branched hydrocarbon chain, wherein one or more hydrogen atoms of the chain are optionally substituted with the same or different 1 to 4 group(s) selected from
(a) a halogen,
(b) a hydroxy,
(c) a C₁₋₆ alkoxy (said group being optionally substituted with the same or different 1 to 3 halogen), and
(d) an oxo;
each R¹ and R³ are independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, C(O)R, —C(O)OR, —OC(O)R, —S(O)R, or —S(O)₂R;
each R is independently
(i) a hydrogen,
(ii) a C₁₋₆ aliphatic (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆alkoxy (said group being optionally substituted with the same or different 1 to 3 halogen),
(d) a hydroxy, and
(e) an oxo), or
(iii) a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano;
R² is selected from
(i) a hydrogen,
(ii) a halogen,
(iii) a hydroxy,
(iv) a cyano,
(v) a C₁₋₆ alkoxy (said group being optionally substituted with the same or different 1 to 3 halogen or hydroxy), or
(vi) a 3-8 membered saturated or partially unsaturated monocyclic carbocyclic ring; a phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with one or more R⁵;
provided that when L¹ is a covalent bond, R² is not hydrogen;
each R⁴ is independently —R;
each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, or —S(O)R;
wherein one or more of {an R¹ and an R²}, {R¹ and an R⁴}, {two instances of R¹} and {two instances of R³} may be taken together with their intervening atoms to form a ring, substituted with q instances of R⁵; wherein said ring is a 3-8 membered saturated or partially unsaturated monocyclic carbocyclic ring; or a 4-8 membered saturated or partially unsaturated monocyclic heterocyclic ring;
m is 0-4;
n is 0-4;
p is 0-2; and
q is 0-5.

2. The compound of claim 1, wherein the compound is a compound of formula I-a, I-b, or I-c: 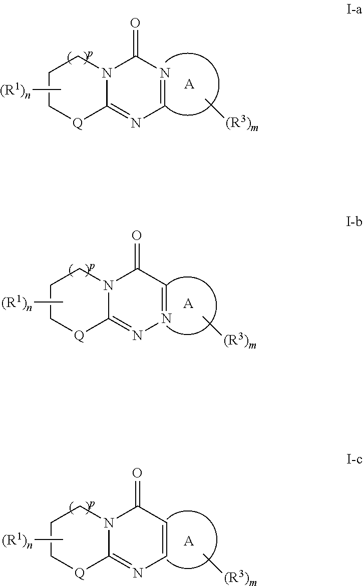
or a pharmaceutically acceptable salt thereof.

3. The compound of claim 1, wherein the compound is a compound of formula II-a, II-b, II-c, II-d, II-e, II-f, II-g, II-h, II-i, II-j, II-k, II-l, II-m, or II-n: 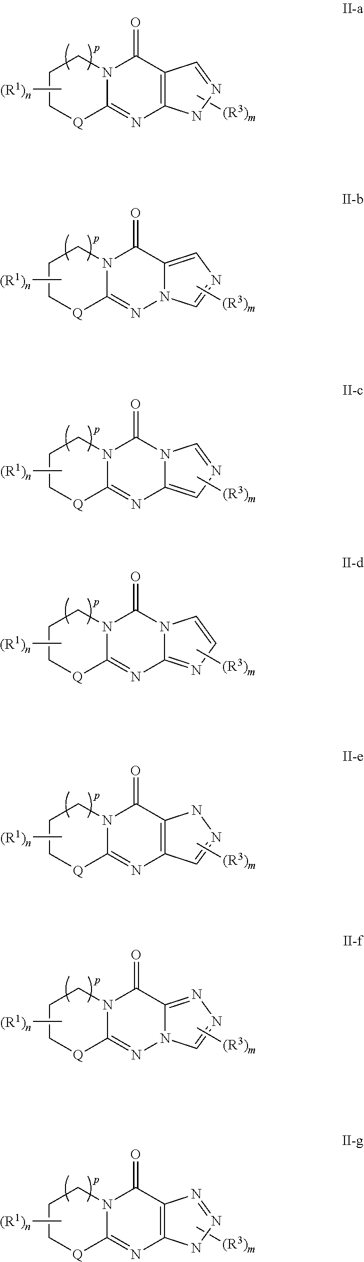 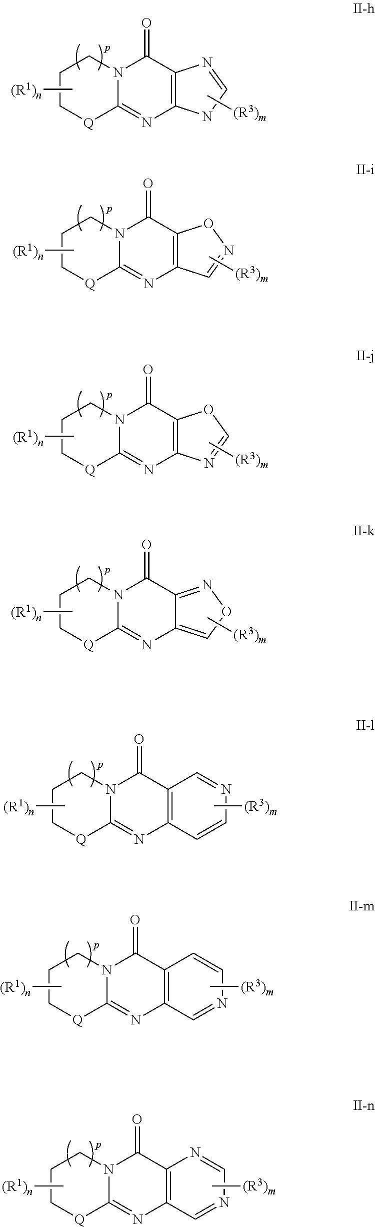
or a pharmaceutically acceptable salt thereof.

4. The compound of claim 1, wherein the compound is a compound of formula II-a, II-b, or II-n: 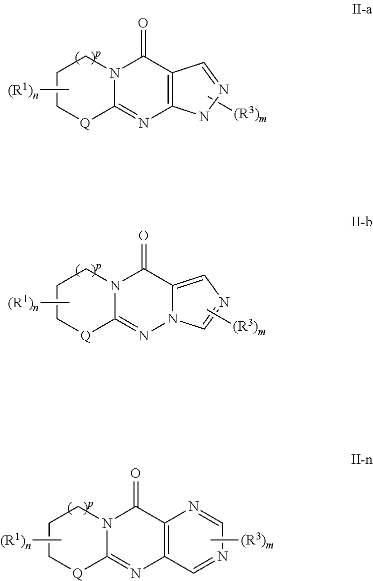
or a pharmaceutically acceptable salt thereof.

5. The compound of claim 1, wherein the compound is a compound of formula III-a, III-b, or III-n: 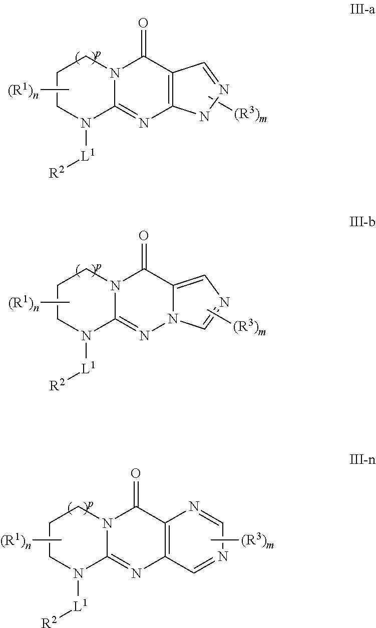
or a pharmaceutically acceptable salt thereof.

6. The compound of claim 1, or a pharmaceutically acceptable salt thereof, wherein each R¹ is independently selected from
(i) a hydrogen,
(ii) a halogen,
(iii) a C₃₋₇ cycloaliphatic; phenyl; a 5 or 6-membered monocyclic heteroaryl, a C₁₋₄alkyl-phenyl, or a C₁₋₄ alkyl-5 or 6-membered monocyclic heteroaryl, each of said group is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano, or
(iv) a C₁₋₆ alkyl (said group being optionally substituted with the same or different 1 to 3 halogen); or two instances of R¹ may be taken together with their intervening atoms to form a 3-6 membered saturated monocyclic carbocyclic ring.

7. The compound of claim 1, or a pharmaceutically acceptable salt thereof, wherein R¹ is selected from
(i) a hydrogen, or
(ii) a phenyl; or a 6 membered monocyclic heretoaryl, each of said group is optionally substituted with the same or different 1 to 4 group(s) selected from the group consisting of
(a) a halogen,
(b) a C₁₋₆ alkyl (said group being optionally substituted with the same or different 1 to 3 halogen), and
(c) a C₁₋₈ alkoxy (said group being optionally substituted with the same or different 1 to 3 halogen).

8. The compound of claim 1, or a pharmaceutically acceptable salt thereof, wherein each R³ is independently selected from
(i) a hydrogen,
(ii) a halogen,
(iii) a C₁₋₆ aliphatic (said group being optionally substituted with the same or different 1 to 4 group(s) selected from
(a) a halogen,
(b) a C₁₋₆ alkoxy (said group being optionally substituted with the same or different 1 to 3 halogen),
(c) a hydroxy, and
(d) an oxo),
(iv) 4-8 membered saturated or partially unsaturated monocyclic heterocyclyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano), or
(v) a cyano.

9. The compound of claim 1, or a pharmaceutically acceptable salt thereof, wherein R³ is selected from
(i) a hydrogen,
(ii) a halogen,
(iii) a C₁₋₆ alkyl (said group being optionally substituted with the same or different 1 to 4 group(s) selected from
(a) a halogen, and
(b) a C₁₋₆alkoxy (said group being optionally substituted with the same or different 1 to 3 halogen)),
(iv) a C₃₋₆cycloalkyl (said group being optionally substituted with the same or different 1 to 4 group(s) selected from
(a) a halogen, and
(b) a C₁ alkyl (said group being optionally substituted with the same or different 1 to 3 halogen)), or
(v) a 4-8 membered saturated or partially unsaturated monocyclic heterocyclyl (said group being optionally substituted with the same or different 1 to 4 group(s) selected from
(a) a halogen, and
(b) a C₁₋₆ alkyl (said group being optionally substituted with the same or different 1 to 3 halogen)).

10. The compound of claim 1, or a pharmaceutically acceptable salt thereof, wherein L¹ is a C₁₋₆ bivalent straight or branched hydrocarbon chain (said group being optionally substituted with an oxo).

11. The compound of claim 1, or a pharmaceutically acceptable salt thereof, wherein R² is selected from
(i) a hydrogen,
(ii) a halogen,
(iii) a hydroxy,
(iv) a C₁₋₆ alkoxy (said group being optionally substituted with the same or different 1 to 3 halogen), or
(v) a C₃₋₇ cycloaliphatic; a phenyl; a 5-6 membered monocyclic heteroaryl, or a 4-8 membered saturated or partially unsaturated monocyclic heterocyclyl, wherein each of said groups is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen or hydroxy),
(d) a hydroxy,
(e) a cyano, and
(f) a 5-6 membered monocyclic heteroaryl (said group being optionally substituted with the same or different 1 to 3 halogen).

12. The compound of claim 1, or a pharmaceutically acceptable salt thereof, wherein R² is a hydrogen or a C₃₋₇ cycloalkyl (said group being optionally substituted with the same or different 1 to 4 halogen, C₁ alkyl (said group being optionally substituted with the same or different 1 to 3 halogen), or C₁₋₆ alkoxy (said group being optionally substituted with the same or different 1 to 3 halogen)).

13. The compound of claim 1, wherein n is 0-1, m is 1, p is 0-1, and q is 0, or a pharmaceutically acceptable salt thereof.

14. A composition comprising a compound according to claim 1 and a pharmaceutically acceptable carrier, adjuvant, or vehicle.

15. A method of inhibiting PDE1 in a patient in need thereof, comprising administering to said patient the composition according to claim 14.

16. A method of inhibiting PDE1 in a biological sample, comprising contacting the biological sample with the compound according to claim 1.

17. A method for treating a neurological or psychiatric disorder in a patient in need thereof, comprising administering to said patient the composition according to claim 14.

18. The method according to claim 17, wherein the neurological or psychiatric disorder is Alzheimer's Disease, Parkinson's Disease, depression, cognitive impairment, stroke, schizophrenia, Down Syndrome, or Fetal Alcohol Syndrome.

19. The method according to claim 17, wherein the neurological or psychiatric disorder involves a deficit in one or more cognitive domains as defined by DSM-5.
